(19) United States
(12) Patent Application Publication
(10) Pub. No.: US 2026/0092037 A1
(43) Pub. Date: Apr. 2, 2026

(54) CRYSTALLINE SALT FORMS OF N-((1-(2-(TERT-BUTYLAMINO)-2-OXOETHYL)PIPERIDIN-4-YL) METHYL)-3-CHLORO-5-FLUOROBENZAMIDE AND METHODS OF USE THEREOF

(71) Applicant: Praxis Precision Medicines, Inc., Boston, MA (US)

(72) Inventors: David T. Jonaitis, Brookston, IN (US); Lisa McCracken, Lafayette, IN (US)

(21) Appl. No.: 19/311,876

(22) Filed: Aug. 27, 2025

Related U.S. Application Data
(63) Continuation of application No. PCT/US2024/017943, filed on Mar. 1, 2024.
(60) Provisional application No. 63/449,467, filed on Mar. 2, 2023.

(51) Int. Cl. C07D 211/26; A61K 31/445; C07C 55/07; C07C 55/08; C07C 55/12; C07C 55/14; C07C 55/20; C07C 57/145; C07C 57/15; C07C 65/11; C07C 307/02; C07C 309/04; C07C 309/05; C07C 309/29; C07C 309/35; C07D 239/557

(52) U.S. Cl. CPC C07D 211/26; A61K 31/445; C07C 55/07; C07C 55/08; C07C 55/12; C07C 55/14; C07C 55/20; C07C 57/145; C07C 57/15; C07C 65/11; C07C 307/02; C07C 309/04; C07C 309/05; C07C 309/29; C07C 309/35; C07D 239/557; C07B 2200/13

(57) ABSTRACT

Described herein, in part, are crystalline and salt forms of N-((1-(2-(tert-butylamino)-2-oxoethyl)piperidin-4-yl)methyl)-3-chloro-5-fluorobenzamide useful for preventing and/or treating a disease or condition relating to aberrant function of a T-type calcium channel, such as epilepsy and epilepsy syndromes (e.g., absence seizures, juvenile myoclonic epilepsy, or a genetic epilepsy), tremor (e.g., essential tremor), and psychiatric disorder (e.g., mood disorders (e.g., major depressive disorder)).

### RELATED APPLICATIONS

This application is a continuation of International Patent Application No. PCT/US2024/017943, filed on Mar. 1, 2024; which claims priority to U.S. Provisional Patent Application No. 63/449,467, filed on Mar. 2, 2023. The entire contents of each of the foregoing applications are hereby incorporated herein by reference.

### BACKGROUND

T-type calcium channels are low-voltage activated ion channels that mediate the influx of calcium into cells. Aberrant function of these ion channels is associated with several diseases or conditions, including psychiatric disorder (e.g., mood disorder), pain, tremor (e.g., essential tremor), epilepsy, or an epilepsy syndrome (e.g., absence seizures and juvenile myoclonic epilepsy). Accordingly, compounds that selectively modulate T-type calcium channels in mammals may be useful in treatment of such diseases or conditions.

N-((1-(2-(tert-butylamino)-2-oxoethyl) piperidin-4-yl)methyl)-3-chloro-5-fluorobenzamide, herein referred to as Compound 1, is a novel selective T-type calcium channel blocker currently in clinical development. Compound 1 is disclosed, e.g., in WO 2021/007487 and has the following structure:

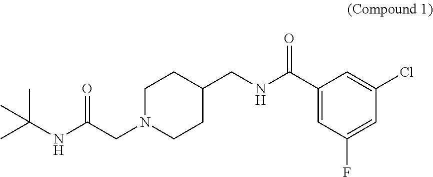 [SMILES: CC(C)(C)NC(=O)CN1CCC(CNC(=O)c2cc(F)cc(Cl)c2)CC1]

Compound 1 is currently being clinically developed for a number of indications, including, e.g., essential tremor. See, e.g., U.S. clinical trials identifier NCT05021978. In view of the clinical importance of Compound 1, there is a need to prepare and characterize different salts of Compound 1, including crystalline forms of salts of Compound 1.

### SUMMARY OF THE INVENTION

Described herein are salts and crystalline forms of Compound 1, wherein the salts and/or crystalline forms are selected from the group consisting of acetate salt, adipate salt, alginate salt, ascorbate salt, asparatate salt, besylate salt, benzoate salt, citrate salt, cyclamate salt, edisylate salt, esylate salt, isethionate salt, fumarate salt, gentisate salt, gluconate salt, glucuronate salt, glutamate salt, glutarate salt, ketoglutarate salt, glycolate salt, hippurate salt, lactobionate salt, maleate salt, malate salt, malonate salt, mesylate salt, napadisylate salt, napsylate salt, oleate salt, oroate salt, oxalate salt, pamoate salt, phosphate salt, sebacate salt, succinate salt and tartrate salt.

In an embodiment, provided herein is a crystalline form of the edisylate salt of compound 1 characterized by an X-ray powder diffraction pattern (XRPD pattern) that includes at least one peak at the diffraction angle (° 2θ) selected from the group of peaks listed in Table 1.

In another embodiment, provided herein is a crystalline form of the esylate salt of compound 1 characterized by an X-ray powder diffraction pattern (XRPD pattern) that includes at least one peak at the diffraction angle (° 2θ) selected from the group of peaks listed in Table 2.

In another embodiment, provided herein is a crystalline form of the glutarate salt of compound 1 characterized by an X-ray powder diffraction pattern (XRPD pattern) that includes at least one peak at the diffraction angle (° 2θ) selected from the group of peaks listed in Table 3.

In another embodiment, provided herein is a crystalline form of the napadisylate salt of compound 1 characterized by an X-ray powder diffraction pattern (XRPD pattern) that includes at least one peak at the diffraction angle (° 2θ) selected from the group of peaks listed in Table 4.

In another embodiment, provided herein is a crystalline form of the napsylate salt of compound 1 characterized by an X-ray powder diffraction pattern (XRPD pattern) that includes at least one peak at the diffraction angle (° 2θ) selected from the group of peaks listed in Table 5.

In another embodiment, provided herein is a crystalline form of the orotate salt of compound 1 characterized by an X-ray powder diffraction pattern (XRPD pattern) that includes at least one peak at the diffraction angle (° 2θ) selected from the group of peaks listed in Table 6.

In another embodiment, provided herein is a crystalline form of the malate salt of compound 1 characterized by an X-ray powder diffraction pattern (XRPD pattern) that includes at least one peak at the diffraction angle (° 2θ) selected from the group of peaks listed in Table 7.

In yet another embodiment, provided herein is a crystalline form of the malonate salt of compound 1 characterized by an X-ray powder diffraction pattern (XRPD pattern) that includes at least one peak at the diffraction angle (° 2θ) selected from the group of peaks listed in Table 8.

In another embodiment, provided herein is a crystalline form of the mesylate salt of compound 1 characterized by an X-ray powder diffraction pattern (XRPD pattern) that includes at least one peak at the diffraction angle (° 2θ) selected from the group of peaks listed in Table 9.

In another embodiment, provided herein is a crystalline form of the pamoate salt of compound 1 characterized by an X-ray powder diffraction pattern (XRPD pattern) that includes at least one peak at the diffraction angle (° 2θ) selected from the group of peaks listed in Table 10.

In another embodiment, provided herein is a crystalline form of the adipate salt of compound 1 characterized by an X-ray powder diffraction pattern (XRPD pattern) that includes at least one peak at the diffraction angle (° 2θ) selected from the group of peaks listed in Table 11.

In another embodiment, provided herein is a crystalline form of the besylate salt of compound 1 characterized by an X-ray powder diffraction pattern (XRPD pattern) that includes at least one peak at the diffraction angle (° 2θ) selected from the group of peaks listed in Table 12.

In another embodiment, provided herein is a crystalline form of the cyclamate salt of compound 1 characterized by an X-ray powder diffraction pattern (XRPD pattern) that includes at least one peak at the diffraction angle (° 2θ) selected from the group of peaks listed in Table 13.

In another embodiment, provided herein is a crystalline form of the fumarate salt of compound 1 characterized by an X-ray powder diffraction pattern (XRPD pattern) that includes at least one peak at the diffraction angle (° 2θ) selected from the group of peaks listed in Table 14.

In another embodiment, provided herein is a crystalline form of the glycolate salt of compound 1 characterized by an X-ray powder diffraction pattern (XRPD pattern) that includes at least one peak at the diffraction angle (° 2θ) selected from the group of peaks listed in Table 15.

In another embodiment, provided herein is a crystalline form of the oxalate salt of compound 1 characterized by an X-ray powder diffraction pattern (XRPD pattern) that includes at least one peak at the diffraction angle (° 2θ) selected from the group of peaks listed in Table 16.

In another embodiment, provided herein is a crystalline form of the phosphate salt of compound 1 characterized by an X-ray powder diffraction pattern (XRPD pattern) that includes at least one peak at the diffraction angle (° 2θ) selected from the group of peaks listed in Table 17.

In another embodiment, provided herein is a crystalline form of the sebacate salt of compound 1 characterized by an X-ray powder diffraction pattern (XRPD pattern) that includes at least one peak at the diffraction angle (° 2θ) selected from the group of peaks listed in Table 18.

Also provided herein are pharmaceutical compositions comprising a salt or crystalline form of Compound 1 described herein and a pharmaceutically acceptable carrier.

Also provided herein are methods for treating a neurological disorder comprising administering a salt or crystalline of Compound 1 described herein or a pharmaceutical composition described herein.

Also provided herein are methods for treating a psychiatric disorder comprising administering a salt or crystalline of Compound 1 described herein or a pharmaceutical composition described herein.

### BRIEF DESCRIPTION OF THE FIGURES

FIG. 1 shows the X-ray powder diffractogram of the edisylate crystalline salt of Compound 1 (Form 1).

FIG. 2 shows the X-ray powder diffractogram of the esylate crystalline salt of Compound 1 (Form 2).

FIG. 3 shows the X-ray powder diffractogram of the glutarate crystalline salt of Compound 1 (Form 3).

FIG. 4 shows the X-ray powder diffractogram of the napadisylate crystalline salt of Compound 1 (Form 4).

FIG. 5 shows the X-ray powder diffractogram of the napsylate crystalline salt of Compound 1 (Form 5).

FIG. 6 shows the X-ray powder diffractogram of the orotate crystalline salt of Compound 1 (Form 6).

FIG. 7 shows the X-ray powder diffractogram of the maleate crystalline salt of Compound 1 (Form 7).

FIG. 8 shows the X-ray powder diffractogram of the malonate crystalline salt of Compound 1 (Form 8).

FIG. 9 shows the X-ray powder diffractogram of the mesylate crystalline salt of Compound 1 (Form 9).

FIG. 10 shows the X-ray powder diffractogram of the pamoate crystalline salt of Compound 1 (Form 10).

FIG. 11 shows the X-ray powder diffractogram of the adipate crystalline salt of Compound 1 (Form 11).

FIG. 12 shows the X-ray powder diffractogram of the besylate crystalline salt of Compound 1 (Form 12).

FIG. 13 shows the X-ray powder diffractogram of the cyclamate crystalline salt of Compound 1 (Form 13).

FIG. 14 shows the X-ray powder diffractogram of the fumarate crystalline salt of Compound 1 (Form 14).

FIG. 15 shows the X-ray powder diffractogram of the glycolate crystalline salt of

### Compound 1 (Form 15).

FIG. 16 shows the X-ray powder diffractogram of the oxalate crystalline salt of Compound 1 (Form 16).

FIG. 17 shows the X-ray powder diffractogram of the phosphate crystalline salt of Compound 1 (Form 17).

FIG. 18 shows the X-ray powder diffractogram of the sebacate crystalline salt of Compound 1 (Form 18).

FIG. 19 shows the results of DSC and TGA analysis of the edisylate crystalline salt of Compound 1 (Form 1).

FIG. 20 shows the results of the NMR analysis of the edisylate crystalline salt of Compound 1 (Form 1).

FIG. 21 shows the results of DSC and TGA analysis of the maleate crystalline salt of Compound 1 (Form 7).

FIG. 22 shows the results of the NMR analysis of the maleate crystalline salt of Compound 1 (Form 7).

FIG. 23 shows the results of DSC and TGA analysis of the malonate crystalline salt of Compound 1 (Form 8).

FIG. 24 shows the results of the NMR analysis of the malonate crystalline salt of Compound 1 (Form 8).

FIG. 25 shows the results of DSC and TGA analysis of the mesylate crystalline salt of Compound 1 (Form 9).

FIG. 26 shows the results of the NMR analysis of the mesylate crystalline salt of Compound 1 (Form 9).

FIG. 27 shows the results of DSC and TGA analysis of the pamoate crystalline salt of Compound 1 (Form 10).

FIG. 28 shows the results of the NMR analysis of the pamoate crystalline salt of Compound 1 (Form 10).

### DETAILED DESCRIPTION OF THE INVENTION

As generally described herein, the present disclosure provides salts, including crystalline salts, of Compound 1. The present disclosure also provides methods for treating neurological disorders, psychiatric disorders, generalized epileptic syndrome with absence seizures, and essential tremor comprising administering a therapeutically effective amount of a salt of Compound 1, including a crystalline salt of Compound 1.

### Definitions

As used herein, the term “anhydrous” means that the referenced crystalline form has substantially no water in the crystal lattice. In one embodiment, the referenced anhydrous crystalline form has, e.g., less than about 0.1% by weight, e.g., less than about 0.09%, less than about 0.08%, less than about 0.07%, less than about 0.06%, less than about 0.05%, less than about 0.04%, less than about 0.03%, less than about 0.02%, or less than about 0.01%, as determined by Karl Fisher analysis.

As used herein, the term “amorphous” refers to a solid that is present in a non-crystalline state or form. Amorphous solids are disordered arrangements of molecules and therefore possess no distinguishable crystal lattice or unit cell and consequently have no definable long range ordering. Instead, at least one broad signal (e.g., at least one halo) may appear in its diffractogram. Broad signals are characteristic of an amorphous solid. Solid state ordering of solids may be determined by standard techniques known in the art, e.g., by X-ray powder diffraction (XRPD) or differential scanning calorimetry (DSC). Amorphous solids can also be differentiated from crystalline solids e.g., by birefringence using polarized light microscopy.

As used herein, the terms “polymorph”, “crystal form”, “crystalline form”, “solid state form” and “Form” interchangeably refer to a solid having a particular molecular packing arrangement in the crystal lattice. Crystalline forms can be identified and distinguished from each other by at least one characterization technique including, e.g., X-ray powder diffraction (XRPD), single crystal X-ray diffraction, differential scanning calorimetry (DSC), dynamic vapor sorption (DVS), and/or thermogravimetric analysis (TGA). Different polymorphs may have different physical properties such as, for example, melting temperature, heat of fusion, solubility, dissolution rate and/or vibrational spectra as a result of different arrangements or conformations of the molecules in the crystal lattice. The differences in physical properties exhibited by different polymorphs may affect parameters important for pharmaceutical substances, such as for example, storage stability, compressibility and density (important in formulation and product manufacturing), and dissolution rate (an important factor in bioavailability).

Differences in stabilities of different polymorphs may also result from differences in chemical reactivity (e.g., different susceptibility to oxidation). Thus, a dosage form comprised of one polymorph may discolor more rapidly than a dosage form comprised of a different polymorph of the same substance. Differences in stabilities of different polymorphs may also result from differences in mechanical properties (e.g., tablets may crumble on storage as a kinetically favored polymorph converts to a thermodynamically more stable polymorph); or from differences in both chemical and mechanical properties (e.g., tablets of one polymorph are more susceptible to breakdown at high humidity). As a result of solubility/dissolution differences, in extreme cases, some polymorphic transitions may result in loss of potency or, at the other extreme, toxicity. In addition, physical properties of the crystal may be important in processing. For example, different polymorphs of the same substances may exhibit differences in their propensity to form solvates or in their particle shape and size distributions, affecting purification (e.g., one polymorph may be more difficult to filter and wash free of impurities than another polymorph).

Polymorphs of a molecule may be obtained by a number of methods, as known in the art. Such methods may include, but are not limited to, melt recrystallization, melt cooling, solvent recrystallization, desolvation, rapid evaporation, rapid cooling, slow cooling, vapor diffusion and sublimation. Techniques for characterizing polymorphs may include, but are not limited to, differential scanning calorimetry (DSC), X-ray powder diffractometry (XRPD), single crystal X-ray diffractometry, vibrational spectroscopy, e.g., IR and Raman spectroscopy, solid state NMR, hot stage optical microscopy, scanning electron microscopy (SEM), electron crystallography and quantitative analysis, particle size analysis (PSA), surface area analysis, solubility studies and dissolution studies. Specifically, XRPD is a technique used to characterize the crystallographic structure, size, and preferred orientation in polycrystalline or powdered solid samples. This diffraction is also used to characterize heterogeneous solid mixtures to determine the percent of crystalline compounds present and can provide structural information on unknown materials.

The term “X-ray powder diffraction pattern, used herein interchangeably with the term “XRPD pattern” or “X-ray powder diffractogram” refers to a graphical representation of the data collected by XRPD analysis.

The term “peak”, as used herein, refers to a peak in the XRPD pattern having an intensity at least about 20%, e.g., at least about 30%, at least about 40%, at least about 50% or at least about 100% greater than the baseline noise.

The terms “approximately” or “about”, as used herein in reference to a peak in an XRPD pattern, refer to the XRPD pattern in which the peak appears within 0.5° 2θ, e.g., within 0.4, 0.3, 0.2, 0.1, 0.05 or 0.01° 2θ of a given ° 2θ value.

As used herein, an XRPD pattern is “substantially the same as an XRPD pattern depicted in [a particular] Figure” when at least about 80% of the peaks, e.g., at least about 85%, at least about 90%, at least about 95%, at least about 98%, or at least about 99% of the peaks in the two diffractograms are the same ±0.5° 2θ. In determining “substantial similarity,” one of ordinary skill in the art will understand that there may be variation in the intensities and/or signal positions in XRPD diffractograms even for the same crystalline form.

As used herein, the term “chemical purity” refers to the extent to which the disclosed form is free from materials having different chemical structures. Chemical purity of the compound in the disclosed crystal forms means the weight of the compound divided by the sum of the weight of the compound plus materials/impurities having different chemical structures multiplied by 100%, i.e., percent by weight. In one embodiment, the compound in the disclosed crystalline forms has a chemical purity of at least about 60%, at least about 70%, at least about 80%, at least about 90%, at least about 95%, or at least about 99% by weight.

As used herein, the terms “crystalline” or “crystalline form” refer to a solid form of a compound, e.g., Compound 1, in which atoms are arranged in regular, repeating patterns. In some embodiments, the term “crystalline” encompasses a polymorphic form or a non-amorphous form of a compound, e.g., Compound 1, without distinction. The crystalline nature of a compound can be confirmed, for example, by examination of the XRPD pattern of the compound. If the XRPD shows sharp intensity peaks in the XRPD then the compound is crystalline.

As used herein, the term “solvate” refers to a crystalline compound wherein a stoichiometric or non-stoichiometric amount of solvent, or mixture of solvents, is incorporated into the crystal structure.

As used herein, the term “hydrate” refers to a crystalline compound where a stoichiometric or non-stoichiometric amount of water is incorporated into the crystal structure. A hydrate is a solvate wherein the solvent incorporated into the crystal structure is water.

The term “anhydrous” when used herein with respect to a compound means substantially no solvent incorporated into the crystal structure.

As used herein, the term “N-((1-(2-(tert-butylamino)-2-oxoethyl) piperidin-4-yl)methyl)-3-chloro-5-fluorobenzamide” is used interchangeably with the term “Compound 1” in reference to a compound of the following structure:

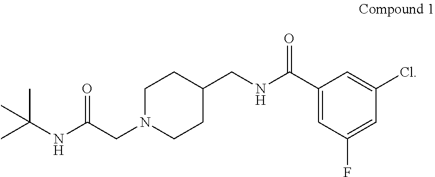 [SMILES: CC(C)(C)NC(=O)CN1CCC(CNC(=O)c2cc(F)cc(Cl)c2)CC1]

The 2-theta (20) values of the X-ray powder diffraction patterns for the crystalline forms described herein may vary slightly from one instrument to another and also depending on variations in sample preparation and batch-to-batch variation. Therefore, unless otherwise defined, the XRPD patterns/assignments recited herein are not to be construed as absolute and can vary ±0.5 degrees.

Temperature values, e.g., for DSC peaks herein may vary slightly from one instrument to another and also depending on variations in sample preparation, batch-to-batch variation, and environmental factors. Therefore, unless otherwise defined, temperature values recited herein are not to be construed as absolute and can vary ±5° C. or ±2° C.

In general, the term “effective amount” of a compound refers to an amount sufficient to elicit the desired biological response. As will be appreciated by those of ordinary skill in this art, the effective amount of a compound of the invention may vary depending on such factors as the desired biological endpoint, the pharmacokinetics of the compound, the disease being treated, the mode of administration, and the age, health, and condition of the subject. An effective amount encompasses therapeutic and prophylactic treatment.

As used herein, and unless otherwise specified, a “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

As used herein, the term “refractory” refers to a disease, disorder, or condition that does not readily yield or respond to therapy or treatment, or is not controlled by a therapy or treatment. In some embodiments, a disease, disorder, or condition described herein is refractory (e.g., refractory epilepsy or refractory absence seizures) and does not respond to standard therapy or treatment.

As used herein, a “subject” to which administration is contemplated includes, but is not limited to, humans (i.e., a male or female of any age group, e.g., a pediatric subject, such as an infant, a child, or an adolescent, or adult subject, such as a young adult, a middle-aged adult or a senior adult) and/or a non-human animal, e.g., a mammal such as a primate (e.g., a cynomolgus monkey or a rhesus monkeys), a cattle, a pig, a horse, a sheep, a goat, a rodent, a cat, and/or a dog. In certain embodiments, the subject is a human. In certain embodiments, the subject is a non-human animal. The terms “human” and “patient” are used interchangeably herein.

The terms “disease”, “disorder”, and “condition” are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition (“therapeutic treatment”), and also contemplates an action that occurs before a subject begins to suffer from the specified disease, disorder or condition (“prophylactic treatment”).

### Salts of Compound 1

Provided herein are salts, including crystalline salts, of Compound 1. Compound 1 is represented by the following structural formula:

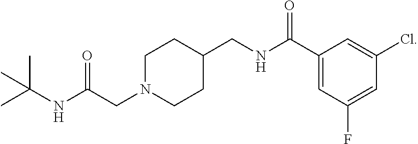 [SMILES: CC(C)(C)NC(=O)CN1CCC(CNC(=O)c2cc(F)cc(Cl)c2)CC1]

In some embodiments, the present disclosure provides salts of Compound 1 selected from the group consisting of acetate salt of Compound 1, adipate salt of Compound 1, alginate salt of Compound 1, ascorbate salt of Compound 1, asparatate salt of Compound 1, besylate salt of Compound 1, benzoate salt of Compound 1, citrate salt of Compound 1, cyclamate salt of Compound 1, edisylate salt of Compound 1, esylate salt of Compound 1, isethionate salt of Compound 1, fumarate salt of Compound 1, gentisate salt of Compound 1, gluconate salt of Compound 1, glucuronate salt of Compound 1, glutamate salt of Compound 1, glutarate salt of Compound 1, ketoglutarate salt of Compound 1, glycolate salt of Compound 1, hippurate salt of Compound 1, lactobionate salt of Compound 1, maleate salt of Compound 1, malate salt of Compound 1, malonate salt of Compound 1, mesylate salt of Compound 1, napadisylate salt of Compound 1, napsylate salt of Compound 1, oleate salt of Compound 1, oroate salt of Compound 1, oxalate salt of Compound 1, pamoate salt of Compound 1, phosphate salt of Compound 1, sebacate salt of Compound 1, succinate salt of Compound 1 and tartrate salt of Compound 1.

In some embodiments, the present disclosure provides salts of Compound 1 selected from the group consisting of adipate salt of Compound 1, besylate salt of Compound 1, cyclamate salt of Compound 1, edisylate salt of Compound 1, esylate salt of Compound 1, isethionate salt of Compound 1, gentisate salt of Compound 1, glucuronate salt of Compound 1, glutamate salt of Compound 1, glutarate salt of Compound 1, ketoglutarate salt of Compound 1, glycolate salt of Compound 1, hippurate salt of Compound 1, mesylate salt of Compound 1, napadisylate salt of Compound 1, napsylate salt of Compound 1, oroate salt of Compound 1 and sebacate salt of Compound 1.

In some embodiments, the present disclosure provides acetate salt of Compound 1.

In some embodiments, the present disclosure provides adipate salt of Compound 1.

In some embodiments, the present disclosure provides alginate salt of Compound 1.

In some embodiments, the present disclosure provides ascorbate salt of Compound 1.

In some embodiments, the present disclosure provides asparatate salt of Compound 1.

In some embodiments, the present disclosure provides besylate salt of Compound 1.

In some embodiments, the present disclosure provides benzoate salt of Compound 1.

In some embodiments, the present disclosure provides citrate salt of Compound 1.

In some embodiments, the present disclosure provides cyclamate salt of Compound 1.

In some embodiments, the present disclosure provides edisylate salt of Compound 1.

In some embodiments, the present disclosure provides esylate salt of Compound 1.

In some embodiments, the present disclosure provides isethionate salt of Compound 1.

In some embodiments, the present disclosure provides fumarate salt of Compound 1.

In some embodiments, the present disclosure provides gentisate salt of Compound 1.

In some embodiments, the present disclosure provides gluconate salt of Compound 1.

In some embodiments, the present disclosure provides glucuronate salt of Compound 1.

In some embodiments, the present disclosure provides glutamate salt of Compound 1.

In some embodiments, the present disclosure provides glutarate salt of Compound 1.

In some embodiments, the present disclosure provides ketoglutarate salt of Compound 1.

In some embodiments, the present disclosure provides glycolate salt of Compound 1.

In some embodiments, the present disclosure provides hippurate salt of Compound 1.

In some embodiments, the present disclosure provides lactobionate salt of Compound 1.

In some embodiments, the present disclosure provides malate salt of Compound 1.

In some embodiments, the present disclosure provides malate salt of Compound 1.

In some embodiments, the present disclosure provides malonate salt of Compound 1.

In some embodiments, the present disclosure provides mesylate salt of Compound 1.

In some embodiments, the present disclosure provides napadisylate salt of Compound 1.

In some embodiments, the present disclosure provides napsylate salt of Compound 1.

In some embodiments, the present disclosure provides oleate salt of Compound 1.

In some embodiments, the present disclosure provides oroate salt of Compound 1.

In some embodiments, the present disclosure provides oxalate salt of Compound 1.

In some embodiments, the present disclosure provides pamoate salt of Compound 1.

In some embodiments, the present disclosure provides phosphate salt of Compound 1.

In some embodiments, the present disclosure provides sebacate salt of Compound 1.

In some embodiments, the present disclosure provides succinate salt of Compound 1.

In some embodiments, the present disclosure provides tartrate salt of Compound 1.

### Crystalline Salt Forms of Compound 1

The present disclosure also provides crystalline forms of a salt of Compound 1. In some embodiments, the salt is selected from the group consisting of adipate salt, besylate salt, cyclamate salt, edisylate salt, esylate salt, fumarate salt, glutarate salt, glycolate salt, napadisylate salt, napsylate salt, orotate salt, oxalate salt, maleate salt, malonate salt, mesylate salt, pamoate salt, phosphate salt, and sebacate salt.

### Crystalline Forms of Edisylate Salt of Compound 1

In some embodiments, the present disclosure provides crystalline forms of edisylate salt of Compound 1.

In some embodiments, the present disclosure provides crystalline Form 1 of edisylate salt of Compound 1 that is characterized by an X-ray powder diffraction pattern (XRPD pattern) that comprises at least one peak, e.g., at least two peaks, at least three peaks, at least four peaks, or at least five or more peaks at the diffraction angle (° 2θ) selected from the group of peaks listed in Table 1.

TABLE 1
°2θ value for edisylate salt

| Position | d-value | Height | Relative Intentity |
|---|---|---|---|
| 9.0400 | 9.7821 | 2894.0000 | 22.9464 |
| 11.0400 | 8.0141 | 5163.0000 | 40.9372 |
| 11.6800 | 7.5764 | 1077.0000 | 8.5395 |
| 11.8600 | 7.4618 | 1308.0000 | 10.3711 |
| 12.3800 | 7.1495 | 924.0000 | 7.3264 |
| 13.0000 | 6.8099 | 1443.0000 | 11.4415 |
| 13.4200 | 6.5977 | 3200.0000 | 25.3727 |
| 13.8600 | 6.3892 | 2231.0000 | 17.6895 |
| 15.3400 | 5.7759 | 2462.0000 | 19.5211 |
| 16.3600 | 5.4181 | 4296.0000 | 34.0628 |
| 17.6000 | 5.0390 | 5043.0000 | 39.9857 |
| 18.0800 | 4.9063 | 1306.0000 | 10.3552 |
| 18.6200 | 4.7652 | 11132.0000 | 88.2651 |
| 19.4800 | 4.5568 | 10398.0000 | 82.4453 |
| 19.8600 | 4.4704 | 2913.0000 | 23.0971 |
| 20.2400 | 4.3873 | 4986.0000 | 39.5338 |
| 20.4800 | 4.3365 | 4148.0000 | 32.8893 |
| 21.0800 | 4.2144 | 12612.0000 | 100.0000 |
| 21.8400 | 4.0694 | 7467.0000 | 59.2055 |
| 23.1000 | 3.8502 | 4230.0000 | 33.5395 |
| 23.8000 | 3.7385 | 1775.0000 | 14.0739 |
| 24.2400 | 3.6717 | 4894.0000 | 38.8043 |
| 24.8200 | 3.5872 | 2005.0000 | 15.8976 |
| 25.4000 | 3.5065 | 1830.0000 | 14.5100 |
| 25.7000 | 3.4663 | 1821.0000 | 14.4386 |
| 26.1000 | 3.4141 | 1720.0000 | 13.6378 |
| 26.4000 | 3.3759 | 1907.0000 | 15.1205 |
| 26.6000 | 3.3510 | 1554.0000 | 12.3216 |
| 27.1400 | 3.2856 | 1659.0000 | 13.1541 |
| 27.8800 | 3.2000 | 4433.0000 | 35.1491 |
| 28.2600 | 3.1578 | 1157.0000 | 9.1738 |
| 28.9400 | 3.0852 | 1681.0000 | 13.3286 |
| 29.7400 | 3.0040 | 1666.0000 | 13.2096 |
| 30.6000 | 2.9215 | 1673.0000 | 13.2651 |
| 31.0800 | 2.8774 | 2291.0000 | 18.1652 |
| 31.7200 | 2.8208 | 807.0000 | 6.3987 |
| 32.4800 | 2.7565 | 1392.0000 | 11.0371 |
| 32.8200 | 2.7288 | 1057.0000 | 8.3809 |
| 33.1800 | 2.7000 | 905.0000 | 7.1757 |
| 34.0200 | 2.6352 | 948.0000 | 7.5167 |
| 35.5400 | 2.5259 | 1052.0000 | 8.3413 |
| 35.9200 | 2.5001 | 1309.0000 | 10.3790 |
| 37.2200 | 2.4157 | 1765.0000 | 13.9946 |
| 37.7000 | 2.3860 | 945.0000 | 7.4929 |
| 38.4000 | 2.3441 | 1050.0000 | 8.3254 |
| 38.9800 | 2.3106 | 954.0000 | 7.5642 |
| 39.6000 | 2.2758 | 1017.0000 | 8.0637 |

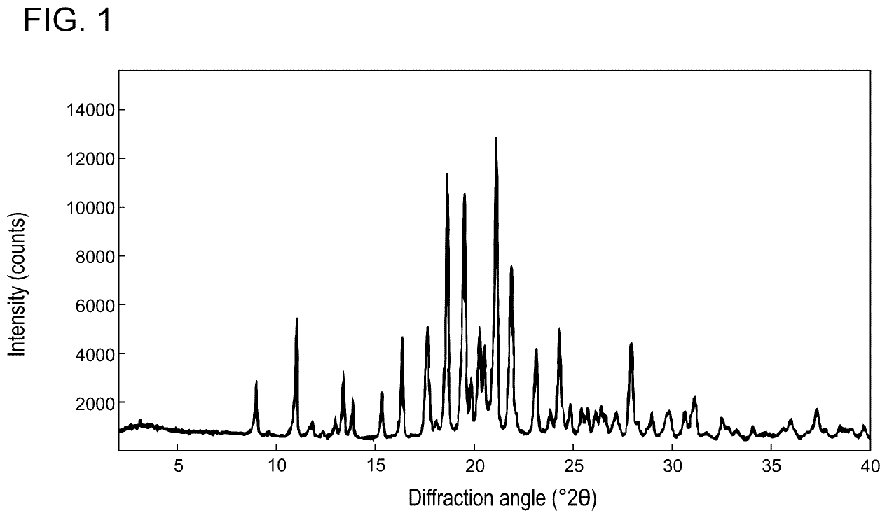

In some embodiments, crystalline Form 1 of edisylate salt of Compound 1 is characterized by an XRPD pattern substantially the same as the XRPD pattern depicted in FIG. 1. In some embodiments, the crystalline form of edisylate salt of Compound 1 is crystalline Form 1 characterized by an XRPD pattern comprising at least one peak, e.g., at least two peaks, at least three peaks, at least four peaks, or at least five or more peaks, at the following 2θ angles (±0.2°): 11.0°, 16.4°, 17.6°, 18.6°, 19.5°, 20.2°, 20.5°, 21.1°, 21.3°, 21.8°, 21.9°, 22.6°, 23.1°, and 38.8°. In another embodiment, the crystalline form of edisylate salt of Compound 1 is crystalline Form 1 characterized by an an XRPD pattern comprising peaks at the following 2θ angles (±0.2°): 18.6°, 19.5°, 21.1°, and 21.8°.

In some embodiments, crystalline Form 1 of edisylate salt of Compound 1 has a melting point as determined by differential scanning calorimetry (DSC) at about 176° C.

In some embodiments, the crystalline Form 1 of edisylate salt of Compound 1 is at least about 60% a single crystalline form, at least about 70% a single crystalline form, at least about 80% a single crystalline form, at least about 90% a single crystalline form, at least about 95% a single crystalline form, or at least about 99% a single crystalline form by weight.

In some embodiments, the crystalline Form 1 of edisylate salt of Compound 1 is substantially free of amorphous forms of Compound 1.

### Crystalline Form of Esylate Salt of Compound 1

In some embodiments, the present disclosure provides crystalline forms of esylate salt of Compound 1.

In some embodiments, the present disclosure provides a crystalline form of esylate salt of Compound 1 that is Crystalline Form 2, characterized by an XRPD pattern that comprises at least one peak, e.g., at least two peaks, at least three peaks, at least four peaks, or at least five or more peaks at the diffraction angle (° 2θ) selected from the group of peaks listed in Table 2.

TABLE 2
°2θ value for esylate salt

| Position | d-value | Height | Relative Intensity |
|---|---|---|---|
| 5.5400 | 15.9518 | 1267.0000 | 2.3930 |
| 6.7000 | 13.1924 | 1114.0000 | 2.1040 |
| 7.3000 | 12.1094 | 1353.0000 | 2.5554 |
| 8.0800 | 10.9421 | 52947.0000 | 100.0000 |
| 10.4600 | 8.4571 | 925.0000 | 1.7470 |
| 11.4400 | 7.7348 | 1957.0000 | 3.6961 |
| 12.9600 | 6.8308 | 1762.0000 | 3.3279 |
| 14.6000 | 6.0670 | 2005.0000 | 3.7868 |
| 15.5600 | 5.6948 | 2405.0000 | 4.5423 |
| 16.1400 | 5.4914 | 10356.0000 | 19.5592 |
| 16.3800 | 5.4115 | 1841.0000 | 3.4771 |
| 17.4400 | 5.0849 | 10773.0000 | 20.3468 |
| 18.0400 | 4.9171 | 977.0000 | 1.8452 |
| 18.6200 | 4.7652 | 1397.0000 | 2.6385 |
| 19.0800 | 4.6514 | 16228.0000 | 30.6495 |
| 19.3600 | 4.5847 | 7272.0000 | 13.7345 |
| 19.9400 | 4.4527 | 6504.0000 | 12.2840 |
| 20.7000 | 4.2909 | 5848.0000 | 11.0450 |
| 20.9800 | 4.2342 | 1577.0000 | 2.9785 |
| 21.2400 | 4.1830 | 3386.0000 | 6.3951 |
| 21.8000 | 4.0768 | 1888.0000 | 3.5658 |
| 22.8600 | 3.8901 | 1934.0000 | 3.6527 |
| 23.4000 | 3.8015 | 2814.0000 | 5.3147 |
| 23.9000 | 3.7231 | 4043.0000 | 7.6359 |
| 24.2400 | 3.6717 | 5286.0000 | 9.9836 |
| 24.7400 | 3.5986 | 2100.0000 | 3.9662 |
| 25.1800 | 3.5367 | 2815.0000 | 5.3166 |
| 25.4000 | 3.5065 | 1311.0000 | 2.4761 |
| 25.7000 | 3.4663 | 1589.0000 | 3.0011 |
| 26.3200 | 3.3860 | 2770.0000 | 5.2316 |
| 26.5400 | 3.3585 | 1324.0000 | 2.5006 |
| 27.2400 | 3.2737 | 1067.0000 | 2.0152 |
| 27.7400 | 3.2158 | 1274.0000 | 2.4062 |
| 29.1400 | 3.0644 | 5830.0000 | 11.0110 |
| 29.5800 | 3.0199 | 1198.0000 | 2.2626 |
| 30.3600 | 2.9440 | 1433.0000 | 2.7065 |
| 30.9200 | 2.8920 | 1363.0000 | 2.5743 |
| 31.2000 | 2.8667 | 957.0000 | 1.8075 |
| 31.8000 | 2.8139 | 2838.0000 | 5.3601 |
| 32.5000 | 2.7549 | 1491.0000 | 2.8160 |
| 33.2000 | 2.6984 | 1194.0000 | 2.2551 |
| 34.1800 | 2.6232 | 959.0000 | 1.8112 |
| 34.8200 | 2.5765 | 1550.0000 | 2.9275 |
| 35.3000 | 2.5425 | 1133.0000 | 2.1399 |
| 35.7800 | 2.5095 | 1814.0000 | 3.4261 |
| 37.3200 | 2.4094 | 1253.0000 | 2.3665 |
| 38.7000 | 2.3266 | 1058.0000 | 1.9982 |

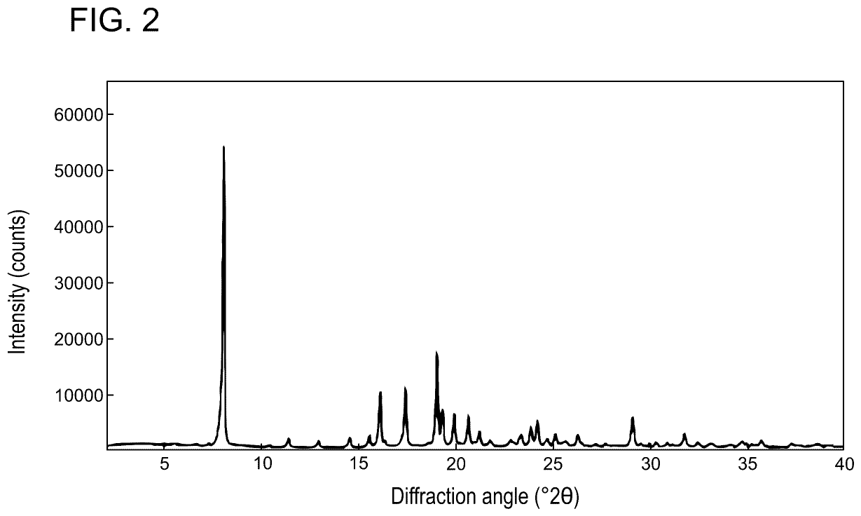

In some embodiments, the crystalline form of esylate salt of Compound 1 is characterized by an XRPD pattern substantially the same as the XRPD pattern depicted in FIG. 2. In some embodiments, the crystalline form of esylate salt of Compound 1 is crystalline Form 2 characterized by an an XRPD pattern comprising at least one peak, e.g., at least two peaks, at least three peaks, at least four peaks, or at least five or more peaks, at the following 2θ angles (±0.2°): 8.1°, 16.1°, 17.4°, 19.1°, 19.4°, 19.9°, 20.7°, and 29.1°. In another embodiment, the crystalline form of esylate salt of Compound 1 is crystalline Form 2 characterized by an an XRPD pattern comprising peaks at the following 2θ angles (±0.2°): 8.1°, 17.4°, and 19.1°.

In some embodiments, the crystalline form of esylate salt of Compound 1 is at least about 60% a single crystalline form, at least about 70% a single crystalline form, at least about 80% a single crystalline form, at least about 90% a single crystalline form, at least about 95% a single crystalline form, or at least about 99% a single crystalline form by weight.

In some embodiments, the crystalline form of esylate salt of Compound 1 is substantially free of amorphous forms of Compound 1.

### Crystalline Forms of Glutarate Salt of Compound 1

In some embodiments, the present disclosure provides crystalline forms of glutarate salt of Compound 1.

In some embodiments, the present disclosure provides crystalline Form 3 of glutarate salt of Compound 1 that is characterized by an XRPD pattern that comprises at least one peak, e.g., at least two peaks, at least three peaks, at least four peaks, or at least five or more peaks at the diffraction angle (° 2θ) selected from the group of peaks listed in Table 3.

TABLE 3
°2θ value for glutarate salt

| Position | d-value | Height | Relative Intensity |
|---|---|---|---|
| 6.9400 | 12.7367 | 8480.0000 | 34.3905 |
| 9.9800 | 8.8628 | 2764.0000 | 11.2093 |
| 12.6600 | 6.9920 | 6409.0000 | 25.9916 |
| 13.1600 | 6.7274 | 1991.0000 | 8.0745 |
| 13.5800 | 6.5203 | 1083.0000 | 4.3921 |
| 14.3600 | 6.1678 | 1747.0000 | 7.0849 |
| 14.8600 | 5.9614 | 1534.0000 | 6.2211 |
| 15.5600 | 5.6948 | 1134.0000 | 4.5989 |
| 16.2600 | 5.4512 | 1406.0000 | 5.7020 |
| 17.4600 | 5.0791 | 1848.0000 | 7.4945 |
| 17.8000 | 4.9829 | 6335.0000 | 25.6915 |
| 18.8000 | 4.7200 | 4835.0000 | 19.6082 |
| 19.0800 | 4.6514 | 9438.0000 | 38.2756 |
| 19.6000 | 4.5291 | 4943.0000 | 20.0462 |
| 20.3200 | 4.3702 | 24658.0000 | 100.0000 |
| 20.7400 | 4.2827 | 8464.0000 | 34.3256 |
| 21.3600 | 4.1598 | 1527.0000 | 6.1927 |
| 21.7400 | 4.0879 | 4859.0000 | 19.7056 |
| 21.9600 | 4.0474 | 3275.0000 | 13.2817 |
| 22.4400 | 3.9619 | 1853.0000 | 7.5148 |
| 23.0600 | 3.8568 | 3553.0000 | 14.4091 |
| 23.3400 | 3.8112 | 2901.0000 | 11.7649 |
| 23.7600 | 3.7447 | 2224.0000 | 9.0194 |
| 24.0200 | 3.7048 | 5189.0000 | 21.0439 |
| 24.5400 | 3.6274 | 3980.0000 | 16.1408 |
| 25.2800 | 3.5229 | 4812.0000 | 19.5150 |
| 25.4400 | 3.5011 | 4374.0000 | 17.7387 |
| 25.8800 | 3.4426 | 1631.0000 | 6.6145 |
| 26.1400 | 3.4089 | 3421.0000 | 13.8738 |
| 26.5200 | 3.3609 | 1993.0000 | 8.0826 |
| 27.3400 | 3.2620 | 2094.0000 | 8.4922 |
| 27.7000 | 3.2204 | 1399.0000 | 5.6736 |
| 28.2800 | 3.1556 | 1408.0000 | 5.7101 |
| 28.7600 | 3.1041 | 3959.0000 | 16.0556 |
| 29.8400 | 2.9941 | 1328.0000 | 5.3857 |
| 30.6400 | 2.9178 | 1595.0000 | 6.4685 |
| 31.0200 | 2.8829 | 2473.0000 | 10.0292 |
| 31.4400 | 2.8453 | 1256.0000 | 5.0937 |
| 32.1800 | 2.7816 | 1436.0000 | 5.8237 |
| 32.6600 | 2.7418 | 1276.0000 | 5.1748 |
| 33.5200 | 2.6734 | 1496.0000 | 6.0670 |
| 34.4200 | 2.6055 | 1139.0000 | 4.6192 |
| 34.7800 | 2.5793 | 1039.0000 | 4.2136 |
| 35.2600 | 2.5453 | 1207.0000 | 4.8950 |
| 35.4400 | 2.5328 | 1107.0000 | 4.4894 |
| 36.0600 | 2.4907 | 1647.0000 | 6.6794 |
| 36.7400 | 2.4461 | 1289.0000 | 5.2275 |
| 37.1200 | 2.4219 | 1344.0000 | 5.4506 |
| 37.4400 | 2.4020 | 1338.0000 | 5.4262 |
| 37.7800 | 2.3811 | 1430.0000 | 5.7993 |
| 38.1000 | 2.3619 | 2473.0000 | 10.0292 |
| 38.5800 | 2.3336 | 1081.0000 | 4.3840 |
| 39.0600 | 2.3060 | 1135.0000 | 4.6030 |
| 39.5800 | 2.2769 | 1180.0000 | 4.7855 |

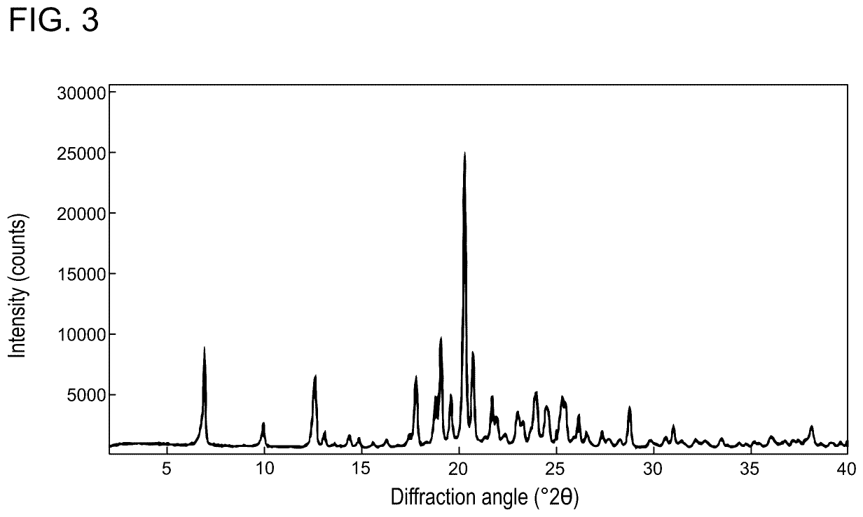

In some embodiments, crystalline Form 3 of glutarate salt of Compound 1 is characterized by an XRPD pattern substantially the same as the XRPD pattern depicted in FIG. 3. In some embodiments, the crystalline form of glutarate salt of Compound 1 is crystalline Form 3 characterized by an XRPD pattern comprising at least one peak, e.g., at least two peaks, at least three peaks, at least four peaks, or at least five or more peaks, at the following 2θ angles (±0.2°): 6.9°, 12.7°, 17.8°, 18.8°, 19.1°, 19.6°, 20.3°, 20.7°, 21.7°, 24.0°, 24.5°, 25.3°, 25.4°, and 28.8°. In another embodiment, the crystalline form of the glutarate salt of Compound 1 is crystalline Form 3 characterized by an XRPD pattern comprising peaks at following 2θ angles (±0.2°): 12.7°, 17.8°, 19.1°, 20.3°, and 20.7°.

In some embodiments, the crystalline Form 3 of glutarate salt of Compound 1 is at least about 60% a single crystalline form, at least about 70% a single crystalline form, at least about 80% a single crystalline form, at least about 90% a single crystalline form, at least about 95% a single crystalline form, or at least about 99% a single crystalline form by weight.

In some embodiments, the crystalline Form 3 of glutarate salt of Compound 1 is substantially free of amorphous forms of Compound 1.

### Crystalline Form of Napadisylate Salt of Compound 1

In some embodiments, the present disclosure provides crystalline forms of napadisylate salt of Compound 1.

In some embodiments, the present disclosure provides a crystalline form of napadisylate salt of Compound 1 that is Crystalline Form 4 characterized by an XRPD pattern that comprises at least one peak, e.g., at least two peaks, at least three peaks, at least four peaks, or at least five or more peaks at the diffraction angle (° 2θ) selected from the group of peaks listed in Table 4.

TABLE 4
°2θ value for napadisylate salt

| Position | d-value | Height | Relative Intensity |
|---|---|---|---|
| 6.4 | 13.8101 | 700.8474 | 3.3026 |
| 7.6 | 11.6935 | 21220.9277 | 100.0000 |
| 10.0 | 8.8805 | 493.2488 | 2.3244 |
| 10.2 | 8.6383 | 1324.6324 | 6.2421 |
| 11.1 | 7.9852 | 415.9244 | 1.9600 |
| 12.1 | 7.3385 | 524.6832 | 2.4725 |
| 12.4 | 7.1151 | 4578.2852 | 21.5744 |
| 13.3 | 6.6569 | 446.1123 | 2.1022 |
| 13.6 | 6.4918 | 596.9269 | 2.8129 |
| 14.7 | 6.0178 | 687.5988 | 3.2402 |
| 15.1 | 5.8595 | 3217.2886 | 15.1609 |
| 15.9 | 5.5599 | 519.1013 | 2.4462 |
| 16.7 | 5.3148 | 602.5036 | 2.8392 |
| 17.3 | 5.1375 | 831.6723 | 3.9191 |
| 17.4 | 5.0965 | 900.1793 | 4.2419 |
| 18.3 | 4.8531 | 1063.2323 | 5.0103 |
| 18.7 | 4.7350 | 531.8021 | 2.5060 |
| 19.2 | 4.6178 | 1192.9048 | 5.6214 |
| 19.6 | 4.5291 | 14100.2256 | 66.4449 |
| 20.0 | 4.4438 | 754.9662 | 3.5576 |
| 20.2 | 4.3959 | 648.4094 | 3.0555 |
| 20.7 | 4.2950 | 1333.5421 | 6.2841 |
| 21.2 | 4.1986 | 1547.7050 | 7.2933 |
| 21.4 | 4.1521 | 1600.9786 | 7.5443 |
| 21.6 | 4.1141 | 2587.6160 | 12.1937 |
| 21.6 | 4.1141 | 2587.6160 | 12.1937 |
| 21.9 | 4.0621 | 744.7073 | 3.5093 |
| 22.4 | 3.9689 | 595.3834 | 2.8056 |
| 22.7 | 3.9171 | 1089.9473 | 5.1362 |
| 23.3 | 3.8176 | 1076.9133 | 5.0748 |
| 23.9 | 3.7170 | 1220.4802 | 5.7513 |
| 24.2 | 3.6806 | 1040.8503 | 4.9048 |
| 24.4 | 3.6421 | 2241.3159 | 10.5618 |
| 24.6 | 3.6216 | 2172.3450 | 10.2368 |
| 25.0 | 3.5645 | 2037.9131 | 9.6033 |
| 25.5 | 3.4957 | 708.7850 | 3.3400 |
| 25.8 | 3.4583 | 623.6691 | 2.9389 |
| 26.8 | 3.3265 | 682.3600 | 3.2155 |
| 27.1 | 3.2951 | 739.6958 | 3.4857 |
| 27.2 | 3.2761 | 807.6981 | 3.8061 |
| 28.0 | 3.1821 | 511.3859 | 2.4098 |
| 28.4 | 3.1383 | 594.3204 | 2.8006 |
| 28.9 | 3.0852 | 374.5040 | 1.7648 |
| 29.4 | 3.0420 | 396.8671 | 1.8702 |
| 29.7 | 3.0079 | 404.0290 | 1.9039 |
| 30.8 | 2.9030 | 380.7592 | 1.7943 |
| 31.2 | 2.8631 | 388.9886 | 1.8330 |
| 33.2 | 2.7016 | 410.8183 | 1.9359 |
| 33.6 | 2.6672 | 437.5262 | 2.0618 |
| 34.8 | 2.5808 | 443.0662 | 2.0879 |
| 38.3 | 2.3488 | 349.6555 | 1.6477 |

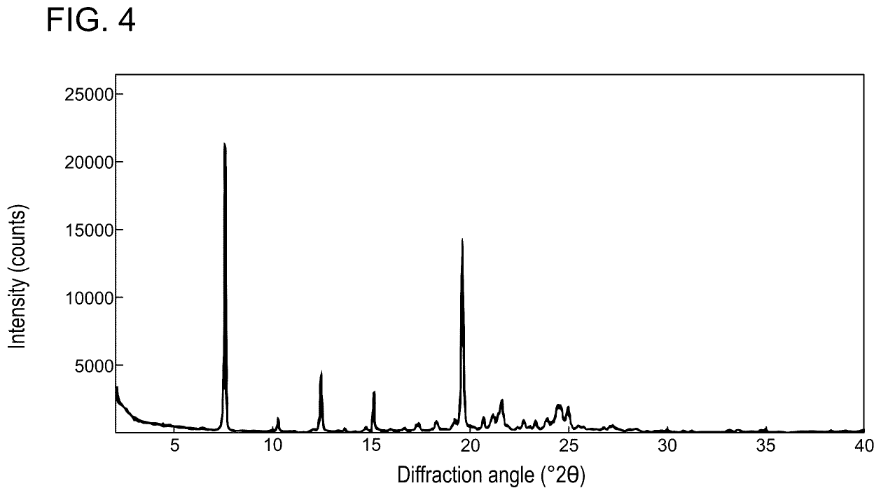

In some embodiments, the crystalline form of napadisylate salt of Compound 1 is characterized by an XRPD pattern substantially the same as the XRPD pattern depicted in FIG. 4. In some embodiments, the crystalline form of napadisylate salt of Compound 1 is crystalline Form 4 characterized by an XRPD pattern comprising at least one peak, e.g., at least two peaks, at least three peaks, at least four peaks, or at least five or more peaks, at the following 2θ angles (±0.2°): 7.6°, 12.4°, 15.1°, 19.6°, 21.6°, 24.4°, 24.6°, and 25.0°. In another embodiment, the crystalline form of napadisylate salt of Compound 1 is crystalline Form 4 characterized by an XRPD spectrum comprising peaks at the following 2θ angles (±0.2°): 7.6°, 12.4°, 15.1°, and 19.6°.

In some embodiments, the crystalline form of napadisylate salt of Compound 1 is at least about 60% a single crystalline form, at least about 70% a single crystalline form, at least about 80% a single crystalline form, at least about 90% a single crystalline form, at least about 95% a single crystalline form, or at least about 99% a single crystalline form by weight.

In some embodiments, the crystalline form of napadisylate salt of Compound 1 is substantially free of amorphous forms of Compound 1.

### Crystalline Form of Napsylate Salt of Compound 1

In some embodiments, the present disclosure provides crystalline forms of napsylate salt of Compound 1.

In some embodiments, the present disclosure provides a crystalline form of napsylate salt of Compound 1 that is Crystalline Form 5 characterized by an XRPD pattern that comprises at least one peak, e.g., at least two peaks, at least three peaks, at least four peaks, or at least five or more peaks at the diffraction angle (° 2θ) selected from the group of peaks listed in Table 5.

TABLE 5
°2θ value for napsylate salt

| Position | d-value | Height | Relative Intensity |
|---|---|---|---|
| 4.5400 | 19.4629 | 1232.0000 | 12.2893 |
| 6.0400 | 14.6324 | 4601.0000 | 45.8953 |
| 7.7000 | 11.4812 | 1336.0000 | 13.3267 |
| 8.3600 | 10.5762 | 937.0000 | 9.3466 |
| 9.5800 | 9.2319 | 864.0000 | 8.6185 |
| 11.0200 | 8.0286 | 1835.0000 | 18.3042 |
| 12.2200 | 7.2427 | 1025.0000 | 10.2244 |
| 13.0800 | 6.7684 | 1151.0000 | 11.4813 |
| 13.4800 | 6.5684 | 1128.0000 | 11.2519 |
| 13.9000 | 6.3709 | 2871.0000 | 28.6384 |
| 14.6000 | 6.0670 | 938.0000 | 9.3566 |
| 15.0400 | 5.8905 | 1199.0000 | 11.9601 |
| 15.3400 | 5.7759 | 3356.0000 | 33.4763 |
| 16.0000 | 5.5391 | 5854.0000 | 58.3940 |
| 16.1600 | 5.4847 | 2445.0000 | 24.3890 |
| 16.6800 | 5.3148 | 1328.0000 | 13.2469 |
| 17.0400 | 5.2033 | 1842.0000 | 18.3741 |
| 17.5000 | 5.0676 | 3389.0000 | 33.8055 |
| 17.9800 | 4.9334 | 2526.0000 | 25.1970 |
| 18.2400 | 4.8636 | 3581.0000 | 35.7207 |
| 18.8000 | 4.7200 | 6324.0000 | 63.0823 |
| 19.1400 | 4.6369 | 1568.0000 | 15.6409 |
| 19.5600 | 4.5383 | 3154.0000 | 31.4613 |
| 19.8000 | 4.4838 | 2146.0000 | 21.4065 |
| 20.0800 | 4.4219 | 2331.0000 | 23.2519 |
| 20.3600 | 4.3617 | 2918.0000 | 29.1072 |
| 20.8600 | 4.2583 | 1658.0000 | 16.5387 |
| 21.1800 | 4.1947 | 3465.0000 | 34.5636 |
| 21.4600 | 4.1406 | 10025.0000 | 100.0000 |
| 21.9400 | 4.0511 | 2073.0000 | 20.6783 |
| 22.5200 | 3.9480 | 5909.0000 | 58.9426 |
| 23.0800 | 3.8535 | 6087.0000 | 60.7182 |
| 23.5600 | 3.7761 | 2436.0000 | 24.2993 |
| 23.9600 | 3.7139 | 1861.0000 | 18.5636 |
| 24.5800 | 3.6216 | 2504.0000 | 24.9776 |
| 25.1000 | 3.5478 | 1587.0000 | 15.8304 |
| 25.4800 | 3.4957 | 1648.0000 | 16.4389 |
| 25.6600 | 3.4716 | 1365.0000 | 13.6160 |
| 26.0400 | 3.4218 | 1837.0000 | 18.3242 |
| 26.2800 | 3.3911 | 1522.0000 | 15.1820 |
| 26.5600 | 3.3560 | 1679.0000 | 16.7481 |
| 27.2200 | 3.2761 | 3022.0000 | 30.1446 |
| 27.5400 | 3.2387 | 1193.0000 | 11.9002 |
| 27.9800 | 3.1888 | 1169.0000 | 11.6608 |
| 28.3600 | 3.1469 | 1209.0000 | 12.0599 |
| 29.1200 | 3.0665 | 1375.0000 | 13.7157 |
| 29.4600 | 3.0319 | 967.0000 | 9.6459 |
| 29.7600 | 3.0020 | 1026.0000 | 10.2344 |
| 30.0000 | 2.9785 | 1024.0000 | 10.2145 |
| 30.2800 | 2.9516 | 1201.0000 | 11.9800 |
| 30.7600 | 2.9066 | 880.0000 | 8.7781 |
| 31.2600 | 2.8613 | 1156.0000 | 11.5312 |
| 31.6000 | 2.8313 | 935.0000 | 9.3267 |
| 32.1000 | 2.7883 | 1050.0000 | 10.4738 |
| 32.4400 | 2.7599 | 1888.0000 | 18.8329 |
| 33.3400 | 2.6874 | 1242.0000 | 12.3890 |
| 34.3400 | 2.6114 | 890.0000 | 8.8778 |
| 34.8600 | 2.5736 | 1012.0000 | 10.0948 |
| 36.9400 | 2.4333 | 1387.0000 | 13.8354 |
| 37.1000 | 2.4232 | 1597.0000 | 15.9302 |
| 37.9400 | 2.3715 | 1270.0000 | 12.6683 |
| 38.8800 | 2.3163 | 960.0000 | 9.5761 |
| 39.3000 | 2.2925 | 1088.0000 | 10.8529 |
| 39.6600 | 2.2725 | 1024.0000 | 10.2145 |

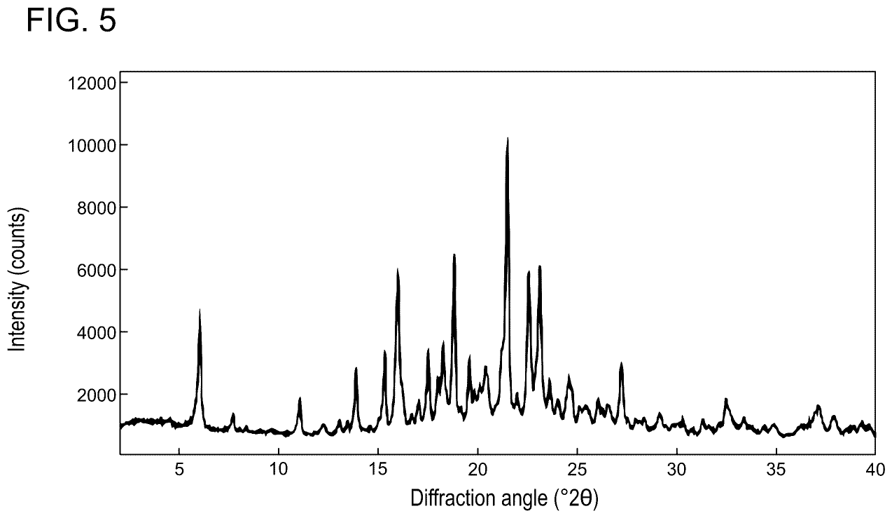

In some embodiments, the crystalline form of napsylate salt of Compound 1 is characterized by an XRPD pattern substantially the same as the XRPD pattern depicted in FIG. 5. In some embodiments, the crystalline form of napsylate salt of Compound 1 is crystalline Form 5 characterized by an XRPD pattern comprising at least one peak, e.g., at least two peaks, at least three peaks, at least four peaks, or at least five or more peaks, at the following 2θ angles (±0.2°): 6.0°, 13.9°, 15.3°, 16.0°, 17.5°, 17.9°, 18.2°, 18.8°, 19.6°, 20.4°, 21.2°, 21.5°, 22.5°, 23.1°, and 27.2°. In another embodiment, the crystalline form of napsylate salt of Compound 1 is crystalline Form 5 characterized by an XRPD pattern comprising peaks at the following 20 angles (±0.2°): 16.0°, 18.8°, 21.5°, 22.5°, and 23.1°.

In some embodiments, the crystalline form of napsylate salt of Compound 1 is at least about 60% a single crystalline form, at least about 70% a single crystalline form, at least about 80% a single crystalline form, at least about 90% a single crystalline form, at least about 95% a single crystalline form, or at least about 99% a single crystalline form by weight.

In some embodiments, the crystalline form of napsylate salt of Compound 1 is substantially free of amorphous forms of Compound 1.

### Crystalline Forms of Orotate Salt of Compound 1

In some embodiments, the present disclosure provides crystalline forms of orotate salt of Compound 1.

In some embodiments, the present disclosure provides crystalline Form 6 of orotate salt of Compound 1 that is Crystalline Form 6 characterized by an XRPD pattern that comprises at least one peak, e.g., at least two peaks, at least three peaks, at least four peaks, or at least five or more peaks at the diffraction angle (° 2θ) selected from the group of peaks listed in Table 6.

TABLE 6
°2θ value for orotate salt

| Position | d-value | Height | Relative Intensity |
|---|---|---|---|
| 8.3400 | 10.6015 | 2535.0000 | 20.8453 |
| 10.9200 | 8.1019 | 5418.0000 | 44.5523 |
| 11.5000 | 7.6945 | 1109.0000 | 9.1193 |
| 12.2400 | 7.2310 | 2750.0000 | 22.6133 |
| 13.1200 | 6.7479 | 1672.0000 | 13.7489 |
| 13.3200 | 6.6470 | 1320.0000 | 10.8544 |
| 13.7200 | 6.4541 | 1231.0000 | 10.1225 |
| 14.0400 | 6.3077 | 1511.0000 | 12.4250 |
| 14.3400 | 6.1764 | 1023.0000 | 8.4121 |
| 15.2200 | 5.8212 | 1331.0000 | 10.9448 |
| 15.9600 | 5.5529 | 1336.0000 | 10.9859 |
| 16.6600 | 5.3212 | 7945.0000 | 65.3318 |
| 16.8800 | 5.2523 | 5096.0000 | 41.9044 |
| 17.1400 | 5.1732 | 4483.0000 | 36.8637 |
| 17.6200 | 5.0334 | 6908.0000 | 56.8045 |
| 18.3800 | 4.8269 | 1956.0000 | 16.0842 |
| 18.9000 | 4.6953 | 7678.0000 | 63.1363 |
| 19.0800 | 4.6514 | 6872.0000 | 56.5085 |
| 19.6600 | 4.5154 | 3811.0000 | 31.3379 |
| 20.0600 | 4.4263 | 3437.0000 | 28.2625 |
| 20.4000 | 4.3533 | 2166.0000 | 17.8110 |
| 20.8000 | 4.2705 | 1520.0000 | 12.4990 |
| 21.6000 | 4.1141 | 2919.0000 | 24.0030 |
| 22.0000 | 4.0402 | 3091.0000 | 25.4173 |
| 22.4200 | 3.9654 | 3073.0000 | 25.2693 |
| 22.9800 | 3.8700 | 5757.0000 | 47.3399 |
| 23.1000 | 3.8502 | 5041.0000 | 41.4522 |
| 24.0000 | 3.7078 | 2802.0000 | 23.0409 |
| 24.1800 | 3.6806 | 2996.0000 | 24.6361 |
| 24.8200 | 3.5872 | 12161.0000 | 100.0000 |
| 25.2200 | 3.5312 | 10269.0000 | 84.4421 |
| 25.7200 | 3.4636 | 2427.0000 | 19.9572 |
| 26.3400 | 3.3835 | 2546.0000 | 20.9358 |
| 26.7400 | 3.3338 | 1382.0000 | 11.3642 |
| 27.0800 | 3.2927 | 1360.0000 | 11.1833 |
| 27.7000 | 3.2204 | 1987.0000 | 16.3391 |
| 28.6200 | 3.1189 | 3729.0000 | 30.6636 |
| 29.2000 | 3.0583 | 3016.0000 | 24.8006 |
| 29.6800 | 3.0099 | 1155.0000 | 9.4976 |
| 30.4800 | 2.9327 | 1676.0000 | 13.7818 |
| 30.6800 | 2.9140 | 1877.0000 | 15.4346 |
| 31.1600 | 2.8702 | 1310.0000 | 10.7721 |
| 32.2400 | 2.7765 | 1880.0000 | 15.4593 |
| 32.5000 | 2.7549 | 1757.0000 | 14.4478 |
| 33.3200 | 2.6890 | 1694.0000 | 13.9298 |
| 33.7600 | 2.6549 | 1485.0000 | 12.2112 |
| 35.0000 | 2.5636 | 1566.0000 | 12.8772 |
| 35.3400 | 2.5397 | 1368.0000 | 11.2491 |
| 35.6600 | 2.5177 | 1818.0000 | 14.9494 |
| 36.0000 | 2.4947 | 1532.0000 | 12.5976 |
| 36.7800 | 2.4436 | 1008.0000 | 8.2888 |
| 38.6400 | 2.3301 | 1668.0000 | 13.7160 |
| 39.4200 | 2.2858 | 1216.0000 | 9.9992 |

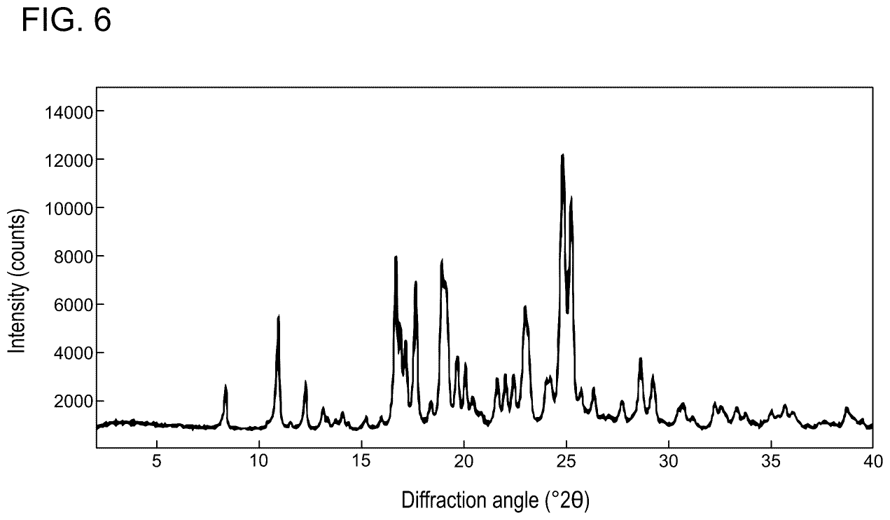

In some embodiments, crystalline Form 6 of orotate salt of Compound 1 is characterized by an XRPD pattern substantially the same as the XRPD pattern depicted in FIG. 6. In some embodiments, the crystalline form of orotate salt of Compound 1 is crystalline Form 6 characterized by an XRPD pattern comprising at least one peak, e.g., at least two peaks, at least three peaks, at least four peaks, or at least five or more peaks, at the following 2θ angles (±0.2°): 10.9°, 16.6°, 16.9°, 17.1°, 17.6°, 18.9°, 19.1°, 19.7°, 20.1°, 22.0°, 22.4°, 23.0°, 23.1°, 24.8°, 25.2°, and 28.6°. In another embodiment, the crystalline form of orotate salt of Compound 1 is crystalline Form 6 characterized by an XRPD pattern comprising peaks at the following 20 angles (±0.2°): 16.6°, 17.6°, 18.9°, 19.1°, 24.8°, 25.2°, and 28.6°.

In some embodiments, the crystalline Form 6 of orotate salt of Compound 1 is at least about 60% a single crystalline form, at least about 70% a single crystalline form, at least about 80% a single crystalline form, at least about 90% a single crystalline form, at least about 95% a single crystalline form, or at least about 99% a single crystalline form by weight.

In some embodiments, the crystalline Form 6 of orotate salt of Compound 1 is substantially free of amorphous forms of Compound 1.

### Crystalline Forms of Maleate Salt of Compound 1

In some embodiments, the present disclosure provides crystalline forms of maleate salt of Compound 1.

In some embodiments, the present disclosure provides crystalline Form 7 of maleate salt of Compound 1 that is characterized by an XRPD pattern that comprises at least one peak, e.g., at least two peaks, at least three peaks, at least four peaks, or at least five or more peaks at the diffraction angle (° 2θ) selected from the group of peaks listed in Table 7.

TABLE 7
°2θ value for maleate salt

| Position | d-value | Height | Relative Intensity |
|---|---|---|---|
| 5.3200 | 16.6110 | 3620.2236 | 83.0515 |
| 10.6200 | 8.3301 | 1190.3066 | 27.3068 |
| 11.7600 | 7.5250 | 783.8263 | 17.9817 |
| 12.1600 | 7.2783 | 1307.0735 | 29.9855 |
| 13.0200 | 6.7995 | 461.5776 | 10.5890 |
| 14.7400 | 6.0097 | 2512.8167 | 57.6465 |
| 15.4400 | 5.7388 | 334.6981 | 7.6783 |
| 15.8800 | 5.5807 | 412.2480 | 9.4574 |
| 16.9600 | 5.2277 | 1455.3320 | 33.3867 |
| 17.1400 | 5.1732 | 730.4818 | 16.7580 |
| 17.7600 | 4.9940 | 1720.6288 | 39.4729 |
| 18.0200 | 4.9225 | 706.1678 | 16.2002 |
| 18.4600 | 4.8062 | 839.9066 | 19.2683 |
| 18.7400 | 4.7350 | 803.5243 | 18.4336 |
| 19.0600 | 4.6562 | 832.5442 | 19.0994 |
| 19.6200 | 4.5246 | 4359.0117 | 100.0000 |
| 20.3800 | 4.3575 | 591.0487 | 13.5592 |
| 20.6400 | 4.3032 | 590.9149 | 13.5562 |
| 20.8800 | 4.2543 | 568.6529 | 13.0455 |
| 21.3000 | 4.1713 | 1866.2568 | 42.8138 |
| 21.5800 | 4.1178 | 4056.8899 | 93.0690 |
| 22.1600 | 4.0114 | 1417.5344 | 32.5196 |
| 23.3200 | 3.8144 | 1219.2693 | 27.9712 |
| 23.7400 | 3.7478 | 2331.7209 | 53.4920 |
| 24.2200 | 3.6746 | 731.1115 | 16.7724 |
| 24.6800 | 3.6072 | 547.5070 | 12.5603 |
| 25.2600 | 3.5257 | 1194.4324 | 27.4014 |
| 25.9000 | 3.4400 | 751.6902 | 17.2445 |
| 26.7400 | 3.3338 | 934.7172 | 21.4433 |
| 27.9800 | 3.1888 | 686.2824 | 15.7440 |
| 29.2600 | 3.0522 | 507.7452 | 11.6482 |
| 30.1400 | 2.9650 | 712.4576 | 16.3445 |
| 30.8800 | 2.8956 | 526.7016 | 12.0831 |
| 31.3200 | 2.8559 | 494.3901 | 11.3418 |
| 31.9200 | 2.8036 | 395.2169 | 9.0667 |
| 32.2200 | 2.7782 | 816.0969 | 18.7221 |
| 33.0000 | 2.7143 | 429.0774 | 9.8435 |
| 34.2800 | 2.6158 | 358.9306 | 8.2342 |
| 35.8600 | 2.5041 | 345.8572 | 7.9343 |
| 36.6000 | 2.4552 | 565.3837 | 12.9705 |
| 38.4400 | 2.3418 | 364.9887 | 8.3732 |
| 39.6400 | 2.2736 | 325.8625 | 7.4756 |

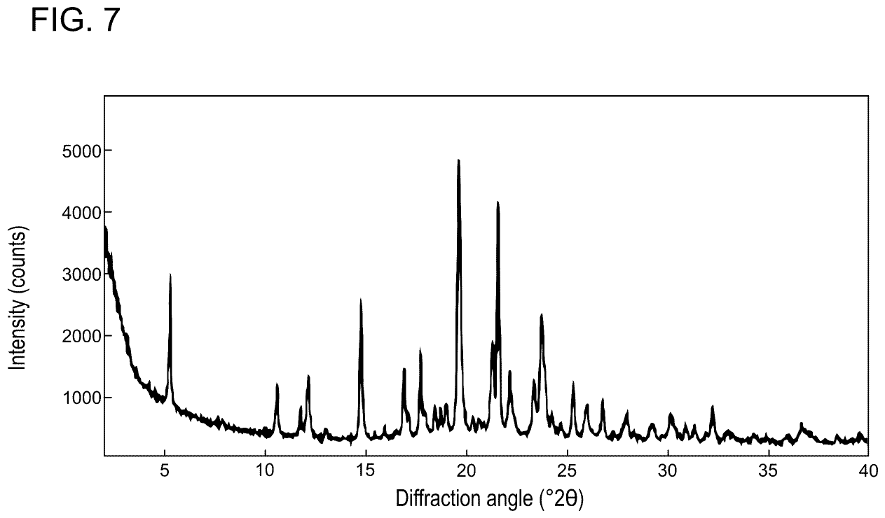

In some embodiments, crystalline Form 7 of maleate salt of Compound 1 is characterized by an XRPD pattern substantially the same as the XRPD pattern depicted in FIG. 7. In some embodiments, the crystalline form of maleate salt of Compound 1 is crystalline Form 7 characterized by an XRPD pattern comprising at least one peak, e.g., at least two peaks, at least three peaks, at least four peaks, or at least five or more peaks, at the following 2θ angles (±0.2°): 5.3°, 10.6°, 12.1°, 14.7°, 17.0°, 17.8°, 19.6°, 21.3°, 21.6°, 22.2°, 23.3°, 23.7°, and 25.3°. In another embodiment, the crystalline form of maleate salt of Compound 1 is crystalline Form 7 characterized by an XRPD pattern comprising peaks at the following 2θ angles (±0.2°): 5.3°, 14.7°, 19.6°, 21.6°, and 23.7°.

In some embodiments, crystalline Form 7 of maleate salt of Compound 1 has a melting point as determined by differential scanning calorimetry (DSC) at about 161° C.

In some embodiments, the crystalline Form 7 of maleate salt of Compound 1 is at least about 60% a single crystalline form, at least about 70% a single crystalline form, at least about 80% a single crystalline form, at least about 90% a single crystalline form, at least about 95% a single crystalline form, or at least about 99% a single crystalline form by weight.

In some embodiments, the crystalline Form 7 of maleate salt of Compound 1 is substantially free of amorphous forms of Compound 1.

### Crystalline Forms of Malonate Salt of Compound 1

In some embodiments, the present disclosure provides crystalline forms of malonate salt of Compound 1.

In some embodiments, the present disclosure provides crystalline Form 8 of malonate salt of Compound 1 that is characterized by an XRPD pattern that comprises at least one peak, e.g., at least two peaks, at least three peaks, at least four peaks, or at least five or more peaks at the diffraction angle (° 2θ) selected from the group of peaks listed in Table 8.

TABLE 8
°2θ value for malonate salt

| Position | d-value | Height | Relative Intensity |
|---|---|---|---|
| 5.5200 | 16.0096 | 6798.8145 | 51.7451 |
| 10.0000 | 8.8451 | 2048.2268 | 15.5888 |
| 10.5600 | 8.3773 | 489.0458 | 3.7221 |
| 10.9200 | 8.1019 | 834.2405 | 6.3493 |
| 11.0800 | 7.9852 | 701.5807 | 5.3397 |
| 11.9400 | 7.4119 | 496.5906 | 3.7795 |
| 12.2200 | 7.2427 | 4173.4717 | 31.7638 |
| 12.6400 | 7.0030 | 1918.3724 | 14.6005 |
| 13.1200 | 6.7479 | 1007.8465 | 7.6706 |
| 14.5600 | 6.0836 | 3608.5232 | 27.4641 |
| 16.1600 | 5.4847 | 845.6823 | 6.4364 |
| 16.4000 | 5.4049 | 4014.0020 | 30.5501 |
| 16.7200 | 5.3022 | 760.9011 | 5.7911 |
| 18.1200 | 4.8956 | 13139.0615 | 100.0000 |
| 18.3000 | 4.8478 | 5584.3120 | 42.5016 |
| 18.9200 | 4.6903 | 1049.3032 | 7.9861 |
| 19.8800 | 4.4660 | 7735.3120 | 58.8726 |
| 20.2800 | 4.3788 | 5571.6021 | 42.4049 |
| 20.7000 | 4.2909 | 581.3895 | 4.4249 |
| 21.1400 | 4.2025 | 3064.6130 | 23.3244 |
| 21.9000 | 4.0584 | 779.0318 | 5.9291 |
| 22.1400 | 4.0149 | 1824.4006 | 13.8853 |
| 22.6000 | 3.9342 | 4773.4277 | 36.3301 |
| 23.6400 | 3.7635 | 1052.2843 | 8.0088 |
| 23.9600 | 3.7139 | 3775.6479 | 28.7361 |
| 24.4400 | 3.6421 | 10258.3545 | 78.0752 |
| 24.9600 | 3.5673 | 1758.8093 | 13.3861 |
| 25.3000 | 3.5202 | 744.9724 | 5.6699 |
| 25.7800 | 3.4557 | 1203.8186 | 9.1621 |
| 26.2400 | 3.3962 | 4289.5610 | 32.6474 |
| 26.5000 | 3.3634 | 2216.8323 | 16.8721 |
| 27.4600 | 3.2480 | 2188.0100 | 16.6527 |
| 28.4800 | 3.1339 | 658.1422 | 5.0091 |
| 29.2000 | 3.0583 | 681.5237 | 5.1870 |
| 29.8800 | 2.9902 | 845.9816 | 6.4387 |
| 30.4000 | 2.9402 | 532.9640 | 4.0563 |
| 30.7000 | 2.9122 | 1078.7883 | 8.2105 |
| 31.0400 | 2.8811 | 985.8402 | 7.5031 |
| 31.3000 | 2.8577 | 1044.8127 | 7.9520 |
| 32.2600 | 2.7748 | 745.5473 | 5.6743 |
| 32.5400 | 2.7516 | 683.1293 | 5.1992 |
| 33.7000 | 2.6595 | 1089.9417 | 8.2954 |
| 34.5200 | 2.5982 | 1822.3644 | 13.8698 |
| 35.2200 | 2.5481 | 840.6662 | 6.3982 |
| 36.1000 | 2.4880 | 708.4379 | 5.3918 |
| 36.6600 | 2.4513 | 1277.0458 | 9.7195 |
| 37.4000 | 2.4045 | 689.8886 | 5.2507 |
| 37.8800 | 2.3751 | 683.4025 | 5.2013 |
| 38.3600 | 2.3465 | 1384.2605 | 10.5355 |
| 39.9200 | 2.2583 | 1134.7308 | 8.6363 |

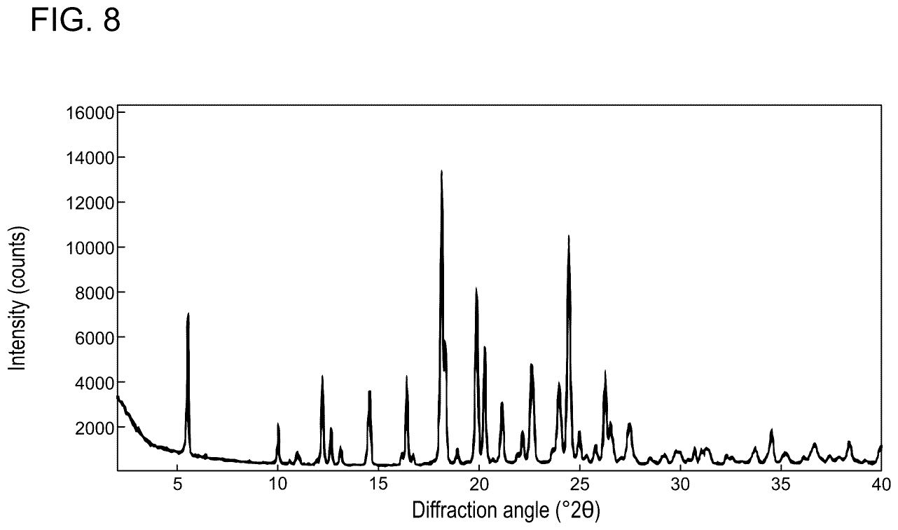

In some embodiments, crystalline Form 8 of malonate salt of Compound 1 is characterized by an XRPD pattern substantially the same as the XRPD pattern depicted in FIG. 8. In some embodiments, the crystalline form of malonate salt of Compound 1 is crystalline Form 8 characterized by an XRPD pattern comprising at least one peak, e.g., at least two peaks, at least three peaks, at least four peaks, or at least five or more peaks, at the following 2θ angles (±0.2°): 5.5°, 12.2°, 14.6°, 16.4°, 18.1°, 18.3°, 19.9°, 20.3°, 22.6°, 24.0°, 24.4°, and 26.2°. In another embodiment, the crystalline form of malonate salt of Compound 1 is crystalline Form 8 characterized by an XRPD pattern comprising peaks at the following 2θ angles (±0.2°): 5.5°, 18.1°, 19.9°, and 24.4°.

In some embodiments, crystalline Form 8 of malonate salt of Compound 1 has a melting point as determined by differential scanning calorimetry (DSC) at about 161° C.

In some embodiments, the crystalline Form 8 of malonate salt of Compound 1 is at least about 60% a single crystalline form, at least about 70% a single crystalline form, at least about 80% a single crystalline form, at least about 90% a single crystalline form, at least about 95% a single crystalline form, or at least about 99% a single crystalline form by weight.

In some embodiments, the crystalline Form 8 of malonate salt of Compound 1 is substantially free of amorphous forms of Compound 1.

### Crystalline Forms of Mesylate Salt of Compound 1

In some embodiments, the present disclosure provides crystalline forms of mesylate salt of Compound 1.

In some embodiments, the present disclosure provides crystalline Form 9 of mesylate salt of Compound 1 that is characterized by an XRPD pattern that comprises at least one peak, e.g., at least two peaks, at least three peaks, at least four peaks, or at least five or more peaks at the diffraction angle (° 2θ) selected from the group of peaks listed in Table 9.

TABLE 9
°2θ value for mesylate salt

| Position | d-value | Height | Relative Intensity |
|---|---|---|---|
| 8.5600 | 10.3295 | 356.6240 | 49.6663 |
| 15.3400 | 5.7759 | 250.1285 | 34.8349 |
| 15.7200 | 5.6372 | 192.3437 | 26.7873 |
| 17.1400 | 5.1732 | 718.0408 | 100.0000 |
| 18.2800 | 4.8531 | 420.8071 | 58.6049 |
| 19.3400 | 4.5894 | 325.0764 | 45.2727 |
| 19.8600 | 4.4704 | 302.5046 | 42.1292 |
| 21.1400 | 4.2025 | 639.0316 | 88.9966 |
| 21.8400 | 4.0694 | 311.2379 | 43.3454 |
| 22.4800 | 3.9550 | 189.4861 | 26.3893 |
| 23.1000 | 3.8502 | 219.0712 | 30.5096 |
| 23.7400 | 3.7478 | 177.7530 | 24.7553 |
| 23.9600 | 3.7139 | 184.5882 | 25.7072 |
| 24.1400 | 3.6866 | 214.4076 | 29.8601 |
| 25.4200 | 3.5038 | 193.4357 | 26.9394 |
| 26.2800 | 3.3911 | 140.1090 | 19.5127 |
| 26.9000 | 3.3143 | 149.6715 | 20.8444 |
| 27.5600 | 3.2364 | 139.8719 | 19.4797 |
| 28.5600 | 3.1253 | 167.9142 | 23.3850 |
| 29.9600 | 2.9824 | 151.3630 | 21.0800 |
| 30.5000 | 2.9308 | 116.1837 | 16.1807 |
| 30.8400 | 2.8993 | 102.3686 | 14.2567 |
| 32.6600 | 2.7418 | 139.2375 | 19.3913 |
| 33.4000 | 2.6827 | 141.1365 | 19.6558 |

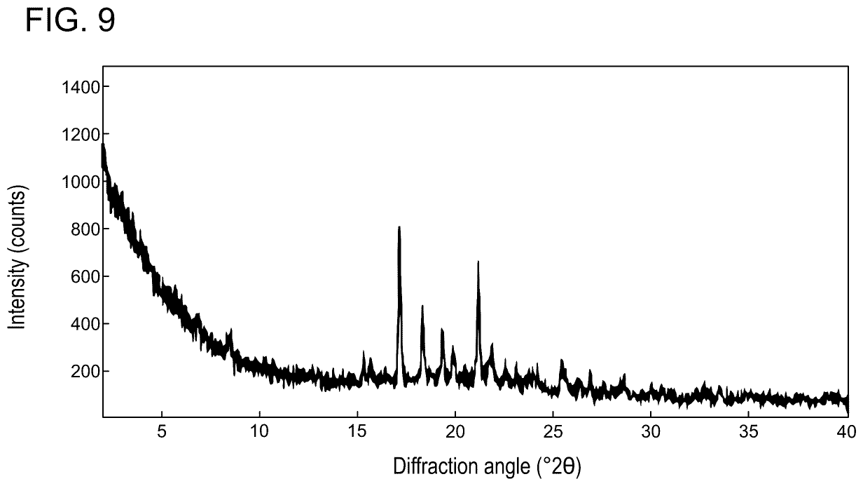

In some embodiments, crystalline Form 9 of mesylate salt of Compound 1 is characterized by an XRPD pattern substantially the same as the XRPD pattern depicted in FIG. 9. In some embodiments, the crystalline form of mesylate salt of Compound 1 is crystalline Form 9 characterized by an XRPD pattern comprising at least one peak, e.g., at least two peaks, at least three peaks, at least four peaks, or at least five or more peaks, at the following 2θ angles (±0.2°): 8.6°, 15.3°, 17.1°, 18.3°, 19.3°, 19.9°, 21.2°, 21.8°, and 23.1°. In another embodiment, the crystalline form of mesylate salt of Compound 1 is crystalline Form 9 characterized by an XRPD pattern comprising peaks at following 2θ angles (±0.2°): 8.6°, 17.1°, 18.3°, and 21.2°.

In some embodiments, crystalline Form 9 of mesylate salt of Compound 1 has a melting point as determined by differential scanning calorimetry (DSC) at about 190° C.

In some embodiments, the crystalline Form 9 of mesylate salt of Compound 1 is at least about 60% a single crystalline form, at least about 70% a single crystalline form, at least about 80% a single crystalline form, at least about 90% a single crystalline form, at least about 95% a single crystalline form, or at least about 99% a single crystalline form by weight.

In some embodiments, the crystalline Form 9 of mesylate salt of Compound 1 is substantially free of amorphous forms of Compound 1.

### Crystalline Forms of Pamoate Salt of Compound 1

In some embodiments, the present disclosure provides crystalline forms of pamoate salt of Compound 1.

In some embodiments, the present disclosure provides crystalline Form 10 of pamoate salt of Compound 1 that is characterized by an XRPD pattern that comprises at least one peak, e.g., at least two peaks, at least three peaks, at least four peaks, or at least five or more peaks at the diffraction angle (° 2θ) selected from the group of peaks listed in Table 10.

TABLE 10
°2θ value for pamoate salt

| Position | d-value | Height | Relative Intensity |
|---|---|---|---|
| 5.9000 | 14.9793 | 9492.3125 | 100.0000 |
| 7.3200 | 12.0763 | 883.3963 | 9.3064 |
| 8.9000 | 9.9357 | 1567.8823 | 16.5174 |
| 9.9800 | 8.8628 | 1543.5585 | 16.2611 |
| 11.2000 | 7.8999 | 3506.2222 | 36.9375 |
| 11.7200 | 7.5506 | 3286.4431 | 34.6222 |
| 13.2200 | 6.6970 | 1792.9785 | 18.8887 |
| 13.4800 | 6.5684 | 1446.3015 | 15.2366 |
| 13.8400 | 6.3984 | 1770.9271 | 18.6564 |
| 14.1400 | 6.2633 | 4053.9761 | 42.7080 |
| 14.5600 | 6.0836 | 1726.0170 | 18.1833 |
| 15.0200 | 5.8983 | 1061.1842 | 11.1794 |
| 15.3200 | 5.7834 | 2067.3503 | 21.7792 |
| 17.6000 | 5.0390 | 2935.2603 | 30.9225 |
| 18.5800 | 4.7754 | 1967.2767 | 20.7249 |
| 18.8000 | 4.7200 | 4060.1526 | 42.7731 |
| 19.0600 | 4.6562 | 1628.3901 | 17.1548 |
| 19.2600 | 4.6083 | 1655.1321 | 17.4366 |
| 19.6400 | 4.5200 | 2947.2029 | 31.0483 |
| 20.1400 | 4.4089 | 5716.4355 | 60.2217 |
| 20.5600 | 4.3198 | 3688.8394 | 38.8613 |
| 20.9800 | 4.2342 | 5971.6030 | 62.9099 |
| 21.9800 | 4.0438 | 2355.1340 | 24.8110 |
| 22.6800 | 3.9205 | 3504.6794 | 36.9212 |
| 23.3800 | 3.8047 | 1269.2156 | 13.3710 |
| 23.7400 | 3.7478 | 3822.9778 | 40.2745 |
| 24.4600 | 3.6391 | 2001.9603 | 21.0903 |
| 24.8200 | 3.5872 | 784.7889 | 8.2676 |
| 25.4000 | 3.5065 | 700.3268 | 7.3778 |
| 25.9800 | 3.4296 | 508.2207 | 5.3540 |
| 26.5800 | 3.3535 | 1071.4956 | 11.2880 |
| 27.0800 | 3.2927 | 804.5250 | 8.4755 |
| 27.4400 | 3.2503 | 1263.7080 | 13.3130 |
| 27.8800 | 3.2000 | 1646.9094 | 17.3499 |
| 28.2000 | 3.1644 | 2014.9738 | 21.2274 |
| 28.8000 | 3.0998 | 611.3683 | 6.4407 |
| 29.6400 | 3.0139 | 1026.2816 | 10.8117 |
| 30.2400 | 2.9554 | 1877.6625 | 19.7809 |
| 31.1000 | 2.8756 | 776.6078 | 8.1814 |
| 31.5600 | 2.8348 | 818.4711 | 8.6225 |
| 31.8600 | 2.8088 | 660.3561 | 6.9567 |
| 32.7400 | 2.7352 | 724.3390 | 7.6308 |
| 34.7400 | 2.5822 | 639.2991 | 6.7349 |
| 35.3800 | 2.5370 | 725.3464 | 7.6414 |
| 36.4600 | 2.4643 | 620.1773 | 6.5335 |
| 37.4600 | 2.4007 | 609.5807 | 6.4218 |
| 38.2200 | 2.3547 | 1096.7999 | 11.5546 |

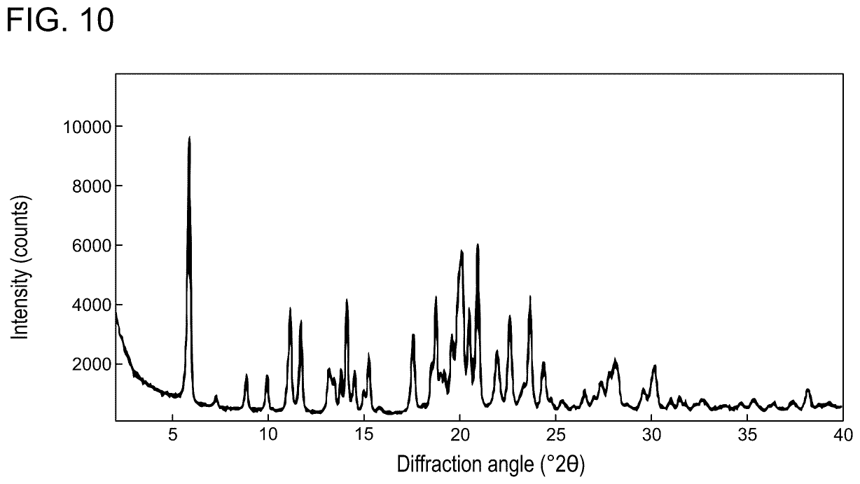

In some embodiments, crystalline Form 10 of pamoate salt of Compound 1 is characterized by an XRPD pattern substantially the same as the XRPD pattern depicted in FIG. 10. In some embodiments, the crystalline form of pamoate salt of Compound 1 is crystalline Form 10 characterized by an XRPD pattern comprising at least one peak, e.g., at least two peaks, at least three peaks, at least four peaks, or at least five or more peaks, at the following 2θ angles (±0.2°): 5.9°, 11.2°, 11.7°, 14.1°, 17.6°, 18.8°, 19.6°, 20.1°, 20.6°, 21.0°, 22.7°, and 23.7°. In another embodiment, the crystalline form of pamoate salt of Compound 1 is crystalline Form 10 characterized by an XRPD pattern comprising peaks at 2θ angles (±0.2°) 5.9°, 14.1°, 18.8°, 20.1°, 21.0°, and 23.7°.

In some embodiments, crystalline Form 10 of pamoate salt of Compound 1 has a melting point as determined by differential scanning calorimetry (DSC) at about 225° C.

In some embodiments, the crystalline Form 10 of pamoate salt of Compound 1 is at least about 60% a single crystalline form, at least about 70% a single crystalline form, at least about 80% a single crystalline form, at least about 90% a single crystalline form, at least about 95% a single crystalline form, or at least about 99% a single crystalline form by weight.

In some embodiments, the crystalline Form 10 of pamoate salt of Compound 1 is substantially free of amorphous forms of Compound 1.

### Crystalline Forms of Adipate Salt of Compound 1

In some embodiments, the present disclosure provides crystalline forms of adipate salt of Compound 1.

In some embodiments, the present disclosure provides crystalline Form 11 of adipate salt of Compound 1 that is characterized by an XRPD pattern that comprises at least one peak, e.g., at least two peaks, at least three peaks, at least four peaks, or at least five or more peaks at the diffraction angle (° 2θ) selected from the group of peaks listed in Table 11.

TABLE 11
°2θ value for adipate salt

| Position | d-value | Height | Relative Intensity |
|---|---|---|---|
| 6.8400 | 12.9227 | 6081.0000 | 46.5656 |
| 9.2200 | 9.5915 | 1525.0000 | 11.6778 |
| 10.0200 | 8.8275 | 984.0000 | 7.5350 |
| 10.5200 | 8.4090 | 3021.0000 | 23.1335 |
| 12.2200 | 7.2427 | 1950.0000 | 14.9322 |
| 12.4000 | 7.1380 | 1802.0000 | 13.7989 |
| 12.6600 | 6.9920 | 1781.0000 | 13.6381 |
| 13.0600 | 6.7787 | 788.0000 | 6.0342 |
| 13.2400 | 6.6870 | 1066.0000 | 8.1630 |
| 13.7000 | 6.4635 | 13059.0000 | 100.0000 |
| 15.1400 | 5.8518 | 969.0000 | 7.4202 |
| 15.5600 | 5.6948 | 1376.0000 | 10.5368 |
| 15.7400 | 5.6301 | 1135.0000 | 8.6913 |
| 16.1200 | 5.4982 | 1357.0000 | 10.3913 |
| 16.3000 | 5.4379 | 1031.0000 | 7.8949 |
| 16.6400 | 5.3275 | 1140.0000 | 8.7296 |
| 16.9800 | 5.2216 | 1679.0000 | 12.8570 |
| 17.2400 | 5.1434 | 1467.0000 | 11.2336 |
| 17.5800 | 5.0447 | 2054.0000 | 15.7286 |
| 17.6800 | 5.0164 | 3603.0000 | 27.5902 |
| 18.4200 | 4.8165 | 9075.0000 | 69.4923 |
| 18.6200 | 4.7652 | 3973.0000 | 30.4235 |
| 19.7200 | 4.5018 | 12865.0000 | 98.5144 |
| 20.5800 | 4.3156 | 8419.0000 | 64.4689 |
| 20.8200 | 4.2664 | 7027.0000 | 53.8096 |
| 21.0600 | 4.2183 | 4718.0000 | 36.1283 |
| 21.2200 | 4.1869 | 4325.0000 | 33.1189 |
| 21.5200 | 4.1292 | 5919.0000 | 45.3251 |
| 22.3400 | 3.9794 | 4792.0000 | 36.6950 |
| 22.9000 | 3.8834 | 7314.0000 | 56.0074 |
| 23.2800 | 3.8208 | 1529.0000 | 11.7084 |
| 24.0200 | 3.7048 | 3773.0000 | 28.8920 |
| 24.2200 | 3.6746 | 4637.0000 | 35.5081 |
| 24.4400 | 3.6421 | 2412.0000 | 18.4700 |
| 24.8400 | 3.5843 | 1241.0000 | 9.5030 |
| 25.5800 | 3.4823 | 5543.0000 | 42.4458 |
| 25.8600 | 3.4452 | 4763.0000 | 36.4729 |
| 26.0400 | 3.4218 | 2779.0000 | 21.2803 |
| 26.2600 | 3.3936 | 1301.0000 | 9.9625 |
| 26.7200 | 3.3362 | 1388.0000 | 10.6287 |
| 27.2400 | 3.2737 | 1719.0000 | 13.1633 |
| 27.5400 | 3.2387 | 4920.0000 | 37.6752 |
| 27.7600 | 3.2136 | 2057.0000 | 15.7516 |
| 28.0600 | 3.1799 | 1421.0000 | 10.8814 |
| 28.2600 | 3.1578 | 1346.0000 | 10.3071 |
| 28.6200 | 3.1189 | 1967.0000 | 15.0624 |
| 28.7400 | 3.1062 | 1849.0000 | 14.1588 |
| 29.0600 | 3.0727 | 1539.0000 | 11.7850 |
| 29.2800 | 3.0501 | 1946.0000 | 14.9016 |
| 29.4800 | 3.0299 | 1555.0000 | 11.9075 |
| 29.7400 | 3.0040 | 1524.0000 | 11.6701 |
| 30.3000 | 2.9497 | 1229.0000 | 9.4111 |
| 30.6400 | 2.9178 | 1131.0000 | 8.6607 |
| 30.9800 | 2.8865 | 1496.0000 | 11.4557 |
| 31.2200 | 2.8649 | 1418.0000 | 10.8584 |
| 32.2800 | 2.7732 | 874.0000 | 6.6927 |
| 32.7800 | 2.7320 | 1360.0000 | 10.4143 |
| 32.9600 | 2.7175 | 1743.0000 | 13.3471 |
| 33.6400 | 2.6641 | 1367.0000 | 10.4679 |
| 34.1400 | 2.6262 | 1056.0000 | 8.0864 |
| 34.3800 | 2.6084 | 1288.0000 | 9.8629 |
| 35.5200 | 2.5273 | 994.0000 | 7.6116 |
| 35.7200 | 2.5136 | 1785.0000 | 13.6687 |
| 36.3400 | 2.4721 | 750.0000 | 5.7432 |
| 36.7400 | 2.4461 | 947.0000 | 7.2517 |
| 37.4000 | 2.4045 | 912.0000 | 6.9837 |
| 37.8400 | 2.3775 | 937.0000 | 7.1751 |
| 38.4000 | 2.3441 | 982.0000 | 7.5197 |
| 38.9600 | 2.3117 | 854.0000 | 6.5396 |
| 39.6000 | 2.2758 | 782.0000 | 5.9882 |

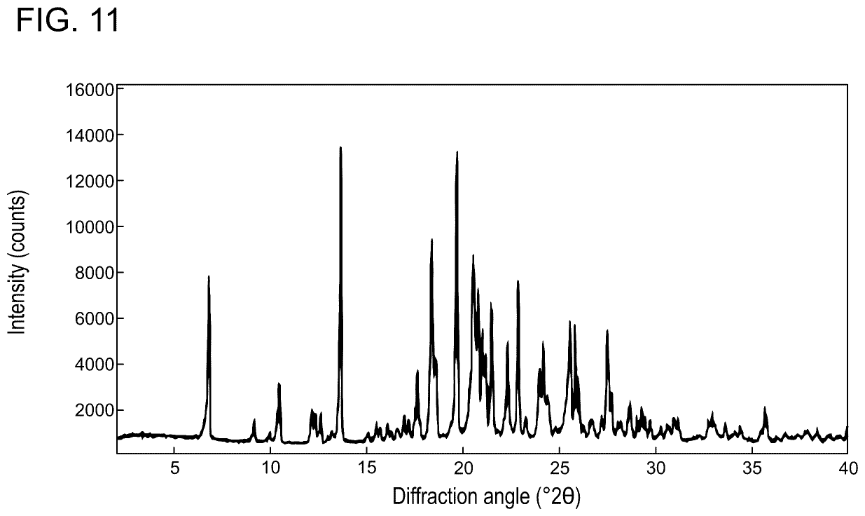

In some embodiments, crystalline Form 11 of adipate salt of Compound 1 is characterized by an XRPD pattern substantially the same as the XRPD pattern depicted in FIG. 11. In some embodiments, the crystalline form of adipate salt of Compound 1 is crystalline Form 11 characterized by an XRPD pattern comprising at least one peak, e.g., at least two peaks, at least three peaks, at least four peaks, or at least five or more peaks, at the following 20 angles (±0.2°): 6.8°, 13.7°, 18.4°, 18.6°, 19.7°, 20.6°, 20.8°, 21.1°, 21.2°, 21.5°, 22.3°, 22.9°, 24.2°, 25.6°, 25.9°, and 27.5°. In another embodiment, the crystalline form of adipate salt of Compound 1 is crystalline Form 11 characterized by an XRPD pattern comprising peaks at the following 2θ angles (±0.2°): 13.7°, 18.4°, 19.7°, 20.6°, 20.8°, and 22.9°.

In some embodiments, the crystalline Form 11 of adipate salt of Compound 1 is at least about 60% a single crystalline form, at least about 70% a single crystalline form, at least about 80% a single crystalline form, at least about 90% a single crystalline form, at least about 95% a single crystalline form, or at least about 99% a single crystalline form by weight.

In some embodiments, the crystalline Form 11 of adipate salt of Compound 1 is substantially free of amorphous forms of Compound 1.

### Crystalline Forms of Besylate Salt of Compound 1

In some embodiments, the present disclosure provides crystalline forms of besylate salt of Compound 1.

In some embodiments, the present disclosure provides crystalline Form 12 of besylate salt of Compound 1 that is characterized by an XRPD pattern that comprises at least one peak, e.g., at least two peaks, at least three peaks, at least four peaks, or at least five or more peaks at the diffraction angle (° 2θ) selected from the group of peaks listed in Table 12.

TABLE 12
°2θ value for besylate salt

| | | | Relative |
|---|---|---|---|
| Position | d-value | Height | Intensity |
| 5.8200 | 15.1850 | 609.2708 | 4.9320 |
| 7.1400 | 12.3804 | 10621.0918 | 85.9770 |
| 9.4600 | 9.3487 | 1145.9053 | 9.2760 |
| 10.2000 | 8.6721 | 421.2856 | 3.4103 |
| 10.2600 | 8.6215 | 565.2717 | 4.5758 |
| 10.5200 | 8.4090 | 805.5078 | 6.5205 |
| 11.1800 | 7.9140 | 3553.8132 | 28.7679 |
| 11.3600 | 7.7890 | 1519.8661 | 12.3032 |
| 12.2200 | 7.2427 | 644.3659 | 5.2161 |
| 12.4000 | 7.1380 | 1345.6495 | 10.8929 |
| 13.2400 | 6.6870 | 292.6049 | 2.3686 |
| 13.6400 | 6.4918 | 418.1405 | 3.3848 |
| 13.8600 | 6.3892 | 785.7319 | 6.3604 |
| 14.2200 | 6.2283 | 1094.6239 | 8.8609 |
| 14.4200 | 6.1423 | 1740.9382 | 14.0928 |
| 14.9200 | 5.9376 | 2489.7996 | 20.1547 |
| 15.3200 | 5.7834 | 5309.5825 | 42.9807 |
| 15.8800 | 5.5807 | 701.0652 | 5.6751 |
| 16.2800 | 5.4445 | 3706.1201 | 30.0008 |
| 16.5800 | 5.3467 | 487.8778 | 3.9493 |
| 17.0200 | 5.2094 | 1403.1097 | 11.3581 |
| 17.2600 | 5.1375 | 822.8193 | 6.6607 |
| 17.4800 | 5.0733 | 5156.5728 | 41.7421 |
| 18.0400 | 4.9171 | 1268.9919 | 10.2724 |
| 18.3600 | 4.8321 | 778.4699 | 6.3017 |
| 18.9000 | 4.6953 | 2828.5488 | 22.8969 |
| 19.3800 | 4.5800 | 457.6616 | 3.7047 |
| 19.9600 | 4.4482 | 2468.4851 | 19.9822 |
| 20.4200 | 4.3491 | 12353.4160 | 100.0000 |
| 20.9800 | 4.2342 | 3893.5303 | 31.5178 |
| 21.4800 | 4.1368 | 2260.1052 | 18.2954 |
| 21.9000 | 4.0584 | 779.9590 | 6.3137 |
| 22.2600 | 3.9936 | 849.9180 | 6.8800 |
| 22.4400 | 3.9619 | 906.6945 | 7.3396 |
| 22.9200 | 3.8800 | 4047.7222 | 32.7660 |
| 23.2400 | 3.8273 | 2674.9851 | 21.6538 |
| 23.7200 | 3.7510 | 1525.8683 | 12.3518 |
| 24.0200 | 3.7048 | 1739.5447 | 14.0815 |
| 24.4800 | 3.6362 | 5191.2324 | 42.0226 |
| 24.8600 | 3.5815 | 787.5301 | 6.3750 |
| 25.2400 | 3.5284 | 1641.7794 | 13.2901 |
| 25.5000 | 3.4930 | 763.2657 | 6.1786 |
| 26.1200 | 3.4115 | 1026.8031 | 8.3119 |
| 26.3600 | 3.3810 | 653.8688 | 5.2930 |
| 26.6800 | 3.3411 | 569.7785 | 4.6123 |
| 26.9600 | 3.3071 | 930.8670 | 7.5353 |
| 27.5000 | 3.2434 | 611.0540 | 4.9464 |
| 27.9200 | 3.1955 | 513.1474 | 4.1539 |
| 28.2000 | 3.1644 | 701.8382 | 5.6813 |
| 28.5200 | 3.1296 | 829.1055 | 6.7115 |
| 29.0200 | 3.0768 | 1610.8196 | 13.0395 |
| 29.2600 | 3.0522 | 907.3018 | 7.3445 |
| 29.6200 | 3.0159 | 1481.3281 | 11.9912 |
| 30.3000 | 2.9497 | 1170.3101 | 9.4736 |
| 30.8800 | 2.8956 | 659.7161 | 5.3404 |
| 31.4800 | 2.8418 | 542.5382 | 4.3918 |
| 32.7800 | 2.7320 | 846.9692 | 6.8562 |
| 33.6800 | 2.6610 | 459.0447 | 3.7159 |
| 33.9800 | 2.6382 | 690.9998 | 5.5936 |
| 34.4200 | 2.6055 | 686.2950 | 5.5555 |
| 34.6400 | 2.5894 | 925.1237 | 7.4888 |
| 35.1400 | 2.5537 | 503.3827 | 4.0748 |
| 35.5600 | 2.5245 | 839.5517 | 6.7961 |
| 36.1800 | 2.4827 | 478.5208 | 3.8736 |
| 36.7400 | 2.4461 | 540.0027 | 4.3713 |
| 37.5600 | 2.3946 | 433.8104 | 3.5117 |
| 38.2200 | 2.3547 | 706.3802 | 5.7181 |
| 39.6000 | 2.2758 | 527.3565 | 4.2689 |

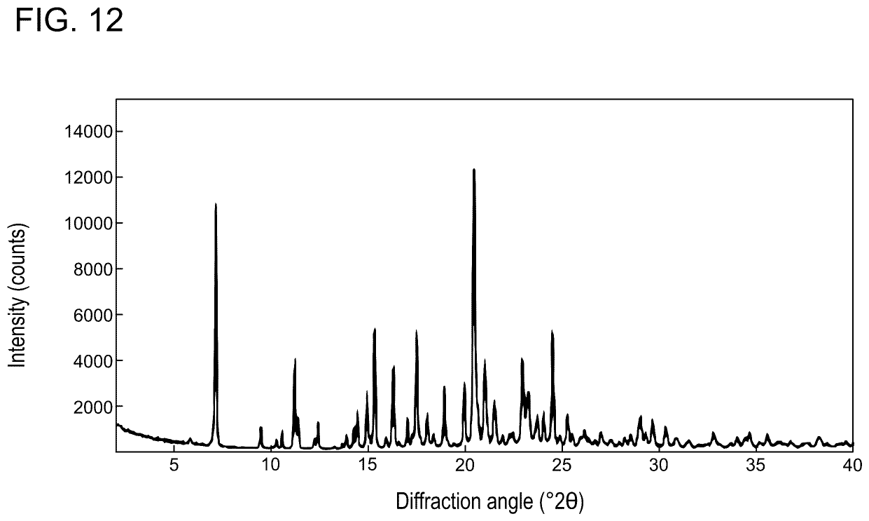

In some embodiments, crystalline Form 12 of besylate salt of Compound 1 is characterized by an XRPD pattern substantially the same as the XRPD pattern depicted in FIG. 12. In some embodiments, the crystalline form of besylate salt of Compound 1 is crystalline Form 12 characterized by an XRPD pattern comprising at least one peak, e.g., at least two peaks, at least three peaks, at least four peaks, or at least five or more peaks, at the following 2θ angles (±0.2°): 7.1°, 15.3°, 16.3°, 17.5°, 20.4°, 21.0°, 22.9°, and 24.5°. In another embodiment, the crystalline form of besylate salt of Compound 1 is crystalline Form 12 characterized by an XRPD pattern comprising peaks at the following 2θ angles (±0.2°): 7.1°, 15.3°, 17.5°, and 20.4°.

In some embodiments, the crystalline Form 12 of besylate salt of Compound 1 is at least about 60% a single crystalline form, at least about 70% a single crystalline form, at least about 80% a single crystalline form, at least about 90% a single crystalline form, at least about 95% a single crystalline form, or at least about 99% a single crystalline form by weight.

In some embodiments, the crystalline Form 12 of besylate salt of Compound 1 is substantially free of amorphous forms of Compound 1.

### Crystalline Forms of Cyclamate Salt of Compound 1

In some embodiments, the present disclosure provides crystalline forms of cyclamate salt of Compound 1.

In some embodiments, the present disclosure provides crystalline Form 13 of cyclamate salt of Compound 1 that is characterized by an XRPD pattern that comprises at least one peak, e.g., at least two peaks, at least three peaks, at least four peaks, or at least five or more peaks at the diffraction angle (° 2θ) selected from the group of peaks listed in Table 13.

TABLE 13
°2θ value for cyclamate salt

| | | | Relative |
|---|---|---|---|
| Position | d-value | Height | Intensity |
| 5.1000 | 17.3270 | 11230.0000 | 71.0849 |
| 9.3000 | 9.5092 | 3821.0000 | 24.1866 |
| 10.1800 | 8.6891 | 15798.0000 | 100.0000 |
| 10.3400 | 8.5550 | 4252.0000 | 26.9148 |
| 10.4800 | 8.4410 | 4321.0000 | 27.3516 |
| 10.9400 | 8.0871 | 1322.0000 | 8.3681 |
| 12.1600 | 7.2783 | 1909.0000 | 12.0838 |
| 12.3800 | 7.1495 | 2675.0000 | 16.9325 |
| 12.8600 | 6.8837 | 1675.0000 | 10.6026 |
| 13.3400 | 6.6371 | 1806.0000 | 11.4318 |
| 13.5600 | 6.5299 | 1424.0000 | 9.0138 |
| 14.2800 | 6.2022 | 3015.0000 | 19.0847 |
| 14.6600 | 6.0423 | 1766.0000 | 11.1786 |
| 14.8800 | 5.9535 | 1716.0000 | 10.8621 |
| 15.4200 | 5.7462 | 2898.0000 | 18.3441 |
| 16.7800 | 5.2834 | 2673.0000 | 16.9199 |
| 17.3600 | 5.1081 | 5924.0000 | 37.4984 |
| 18.2000 | 4.8742 | 3002.0000 | 19.0024 |
| 18.5400 | 4.7856 | 3805.0000 | 24.0853 |
| 19.0400 | 4.6611 | 4099.0000 | 25.9463 |
| 19.3800 | 4.5800 | 2827.0000 | 17.8947 |
| 19.5400 | 4.5429 | 2735.0000 | 17.3123 |
| 20.0000 | 4.4394 | 4292.0000 | 27.1680 |
| 20.4000 | 4.3533 | 3641.0000 | 23.0472 |
| 20.7600 | 4.2786 | 7547.0000 | 47.7719 |
| 21.4400 | 4.1444 | 4287.0000 | 27.1363 |
| 21.9000 | 4.0584 | 3620.0000 | 22.9143 |
| 22.3800 | 3.9724 | 3348.0000 | 21.1926 |
| 22.7200 | 3.9137 | 3110.0000 | 19.6860 |
| 23.2800 | 3.8208 | 3611.0000 | 22.8573 |
| 23.9400 | 3.7170 | 3315.0000 | 20.9837 |
| 24.4600 | 3.6391 | 2861.0000 | 18.1099 |
| 24.8400 | 3.5843 | 3015.0000 | 19.0847 |
| 25.1800 | 3.5367 | 2139.0000 | 13.5397 |
| 25.6000 | 3.4796 | 5234.0000 | 33.1308 |
| 25.9600 | 3.4322 | 2112.0000 | 13.3688 |
| 26.4000 | 3.3759 | 2005.0000 | 12.6915 |
| 27.1600 | 3.2832 | 2087.0000 | 13.2105 |
| 27.9200 | 3.1955 | 2424.0000 | 15.3437 |
| 28.2800 | 3.1556 | 2364.0000 | 14.9639 |
| 28.6600 | 3.1147 | 1401.0000 | 8.8682 |
| 29.2600 | 3.0522 | 2028.0000 | 12.8371 |
| 29.6200 | 3.0159 | 1617.0000 | 10.2355 |
| 31.3000 | 2.8577 | 1645.0000 | 10.4127 |
| 32.2600 | 2.7748 | 1523.0000 | 9.6405 |
| 32.9400 | 2.7191 | 1890.0000 | 11.9635 |
| 34.0200 | 2.6352 | 1827.0000 | 11.5648 |
| 34.6400 | 2.5894 | 1407.0000 | 8.9062 |
| 36.1400 | 2.4853 | 2007.0000 | 12.7041 |
| 36.7200 | 2.4474 | 1505.0000 | 9.5265 |
| 38.0400 | 2.3655 | 1741.0000 | 11.0204 |
| 38.7400 | 2.3243 | 1233.0000 | 7.8048 |
| 39.6800 | 2.2714 | 1272.0000 | 8.0517 |

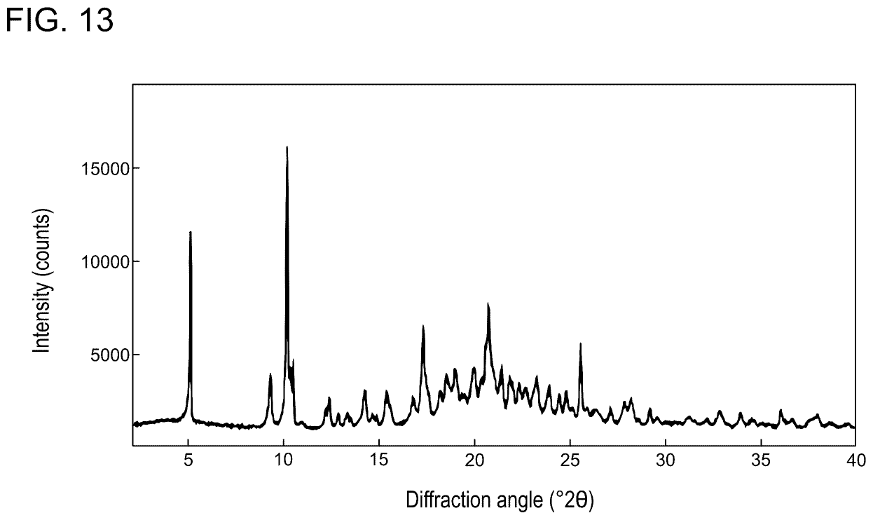

In some embodiments, crystalline Form 13 of cyclamate salt of Compound 1 is characterized by an XRPD pattern substantially the same as the XRPD pattern depicted in FIG. 13. In some embodiments, the crystalline form of cyclamate salt of Compound 1 is crystalline Form 13 characterized by an XRPD pattern comprising at least one peak, e.g., at least two peaks, at least three peaks, at least four peaks, or at least five or more peaks, at the following 2θ angles (±0.2°): 5.1°, 9.3°, 10.2°, 10.3°, 10.5°, 17.4°, 18.5°, 19.0°, 20.0°, 20.4°, 20.8°, 21.4°, 21.9°, 22.4°, and 25.6°. In another embodiment, the crystalline form of cyclamate salt of Compound 1 is crystalline Form 13 characterized by an XRPD pattern comprising peaks at 2θ angles (±0.2°) 5.1°, 10.2°, 17.4°, 20.8°, and 25.6°.

In some embodiments, the crystalline Form 13 of cyclamate salt of Compound 1 is at least about 60% a single crystalline form, at least about 70% a single crystalline form, at least about 80% a single crystalline form, at least about 90% a single crystalline form, at least about 95% a single crystalline form, or at least about 99% a single crystalline form by weight.

In some embodiments, the crystalline Form 13 of cyclamate salt of Compound 1 is substantially free of amorphous forms of Compound 1.

### Crystalline Forms of Fumarate Salt of Compound 1

In some embodiments, the present disclosure provides crystalline forms of fumarate salt of Compound 1.

In some embodiments, the present disclosure provides crystalline Form 14 of fumarate salt of Compound 1 that is characterized by an XRPD pattern that comprises at least one peak, e.g., at least two peaks, at least three peaks, at least four peaks, or at least five or more peaks at the diffraction angle (° 2θ) selected from the group of peaks listed in Table 14.

TABLE 14
°2θ value for fumarate salt

| | | | Relative |
|---|---|---|---|
| Position | d-value | Height | Intensity |
| 5.1000 | 17.3270 | 1982.0000 | 15.8269 |
| 10.1400 | 8.7233 | 2643.0000 | 21.1052 |
| 10.3000 | 8.5881 | 3918.0000 | 31.2864 |
| 10.5400 | 8.3931 | 1155.0000 | 9.2230 |
| 11.7800 | 7.5123 | 2748.0000 | 21.9436 |
| 12.3600 | 7.1610 | 2149.0000 | 17.1604 |
| 12.8600 | 6.8837 | 1955.0000 | 15.6113 |
| 13.2000 | 6.7071 | 2617.0000 | 20.8975 |
| 14.5400 | 6.0919 | 1989.0000 | 15.8828 |
| 14.8000 | 5.9855 | 1181.0000 | 9.4306 |
| 15.1400 | 5.8518 | 1838.0000 | 14.6770 |
| 15.8400 | 5.5947 | 1842.0000 | 14.7089 |
| 16.4800 | 5.3789 | 4709.0000 | 37.6028 |
| 18.0000 | 4.9279 | 12523.0000 | 100.0000 |
| 18.3400 | 4.8373 | 5653.0000 | 45.1409 |
| 18.5000 | 4.7959 | 7826.0000 | 62.4930 |
| 18.9600 | 4.6805 | 2216.0000 | 17.6954 |
| 19.3200 | 4.5941 | 1374.0000 | 10.9718 |
| 19.6800 | 4.5109 | 1467.0000 | 11.7144 |
| 20.0200 | 4.4350 | 3928.0000 | 31.3663 |
| 20.2000 | 4.3959 | 7422.0000 | 59.2669 |
| 20.7400 | 4.2827 | 5385.0000 | 43.0009 |
| 22.4600 | 3.9584 | 7572.0000 | 60.4647 |
| 22.7400 | 3.9103 | 3237.0000 | 25.8484 |
| 23.4600 | 3.7919 | 3074.0000 | 24.5468 |
| 24.0600 | 3.6987 | 5362.0000 | 42.8172 |
| 24.3200 | 3.6598 | 5698.0000 | 45.5003 |
| 24.6600 | 3.6101 | 2692.0000 | 21.4964 |
| 24.6600 | 3.6101 | 2692.0000 | 21.4964 |
| 25.3800 | 3.5093 | 3866.0000 | 30.8712 |
| 26.0600 | 3.4192 | 2300.0000 | 18.3662 |
| 26.4800 | 3.3659 | 3770.0000 | 30.1046 |
| 26.9000 | 3.3143 | 1797.0000 | 14.3496 |
| 27.6600 | 3.2250 | 3144.0000 | 25.1058 |
| 29.3200 | 3.0460 | 1960.0000 | 15.6512 |
| 29.6400 | 3.0139 | 2222.0000 | 17.7434 |
| 30.5200 | 2.9290 | 1578.0000 | 12.6008 |
| 30.8200 | 2.9011 | 1135.0000 | 9.0633 |
| 31.3000 | 2.8577 | 1188.0000 | 9.4865 |
| 31.7000 | 2.8226 | 1544.0000 | 12.3293 |
| 32.5200 | 2.7532 | 1283.0000 | 10.2451 |
| 32.9000 | 2.7223 | 1360.0000 | 10.8600 |
| 34.1800 | 2.6232 | 1301.0000 | 10.3889 |
| 34.4200 | 2.6055 | 1349.0000 | 10.7722 |
| 35.6200 | 2.5204 | 1061.0000 | 8.4724 |
| 36.3600 | 2.4708 | 1083.0000 | 8.6481 |
| 36.9000 | 2.4359 | 922.0000 | 7.3625 |
| 37.4400 | 2.4020 | 1143.0000 | 9.1272 |
| 37.9200 | 2.3727 | 1189.0000 | 9.4945 |
| 38.5000 | 2.3383 | 1068.0000 | 8.5283 |
| 38.6800 | 2.3278 | 1113.0000 | 8.8876 |
| 39.1200 | 2.3026 | 1113.0000 | 8.8876 |
| 39.5400 | 2.2791 | 1532.0000 | 12.2335 |

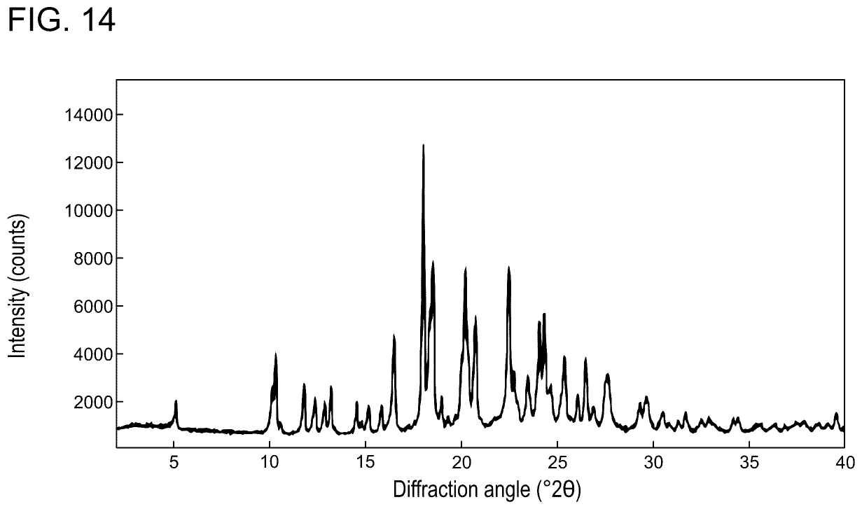

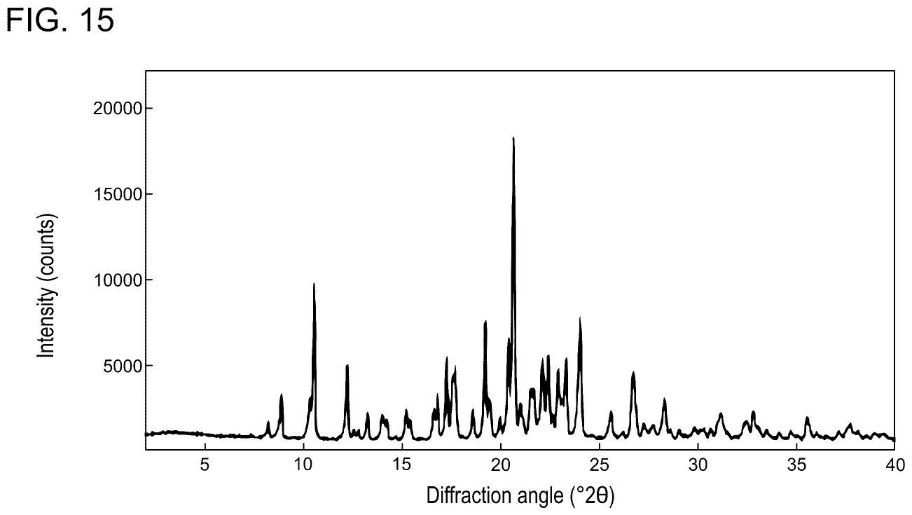

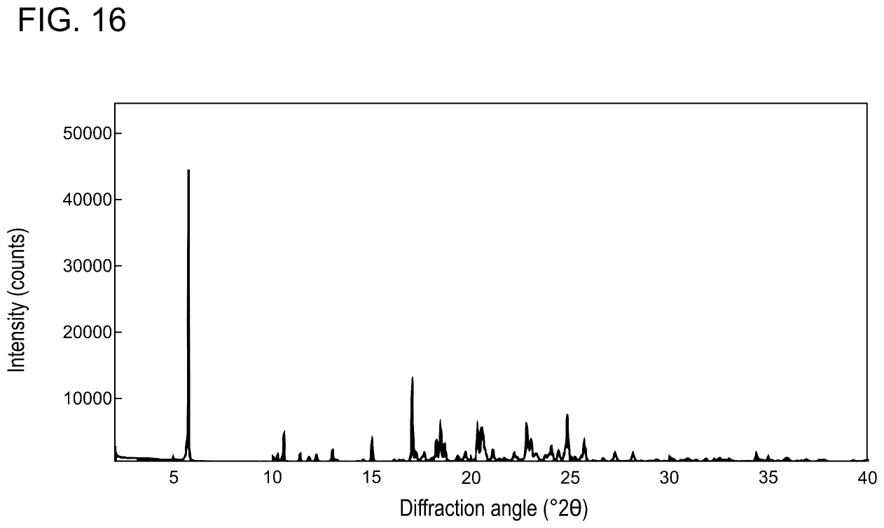

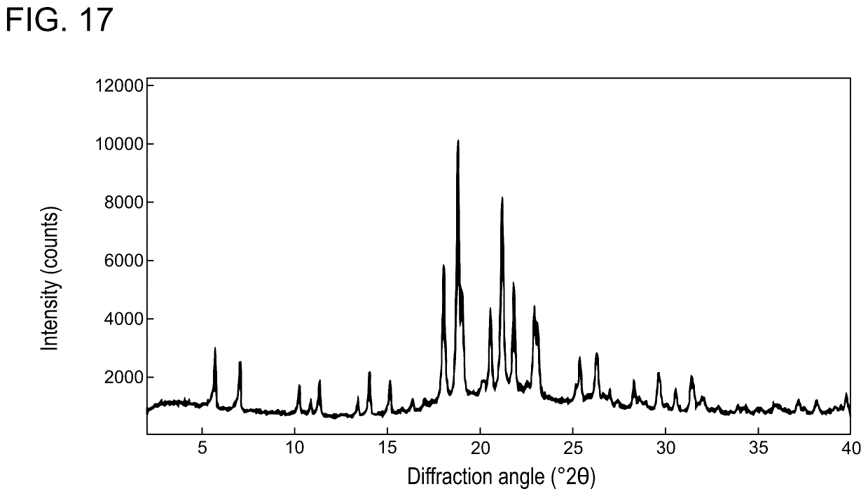

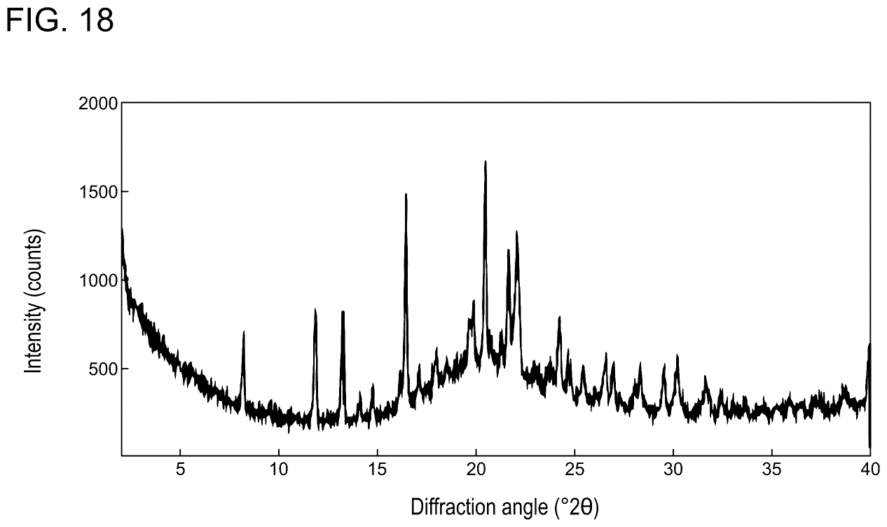

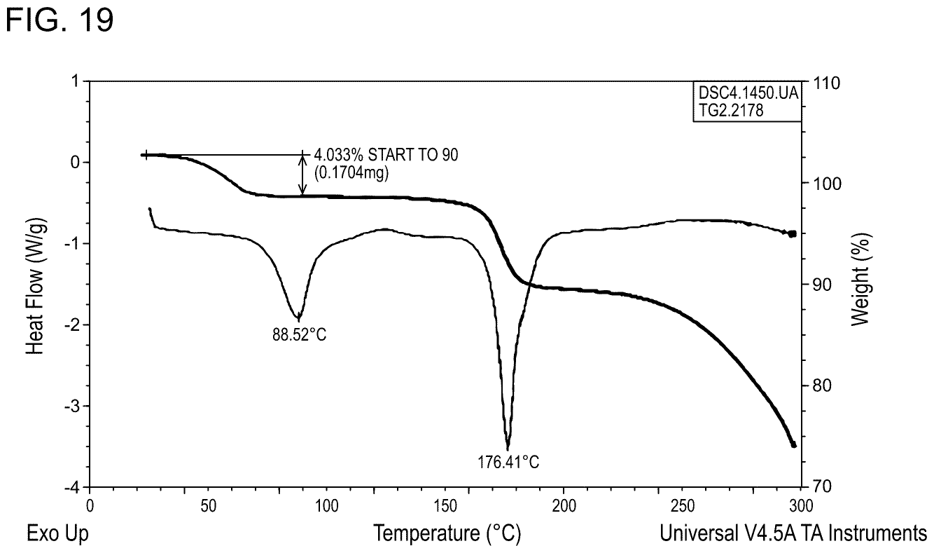

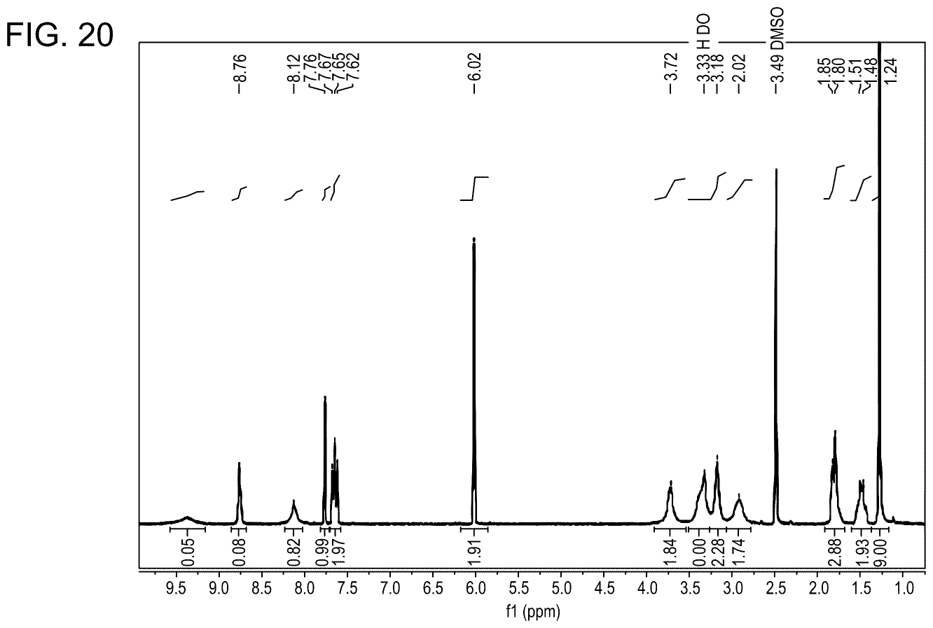

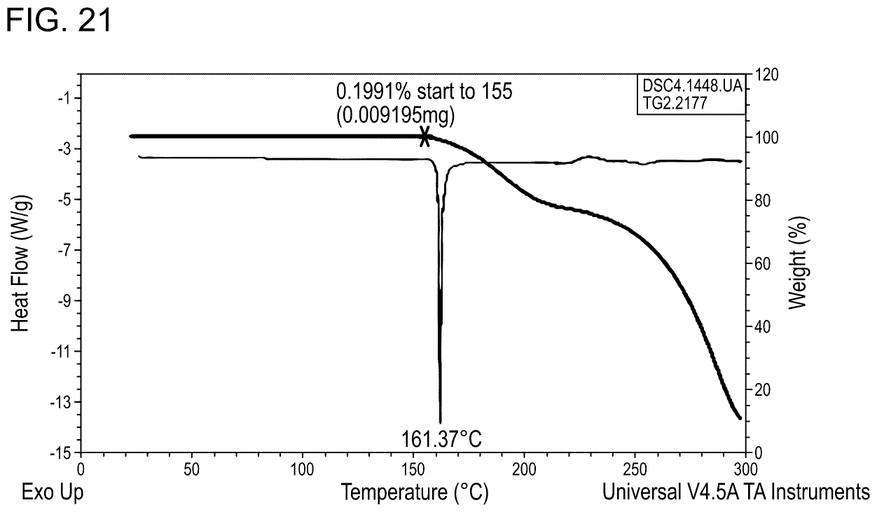

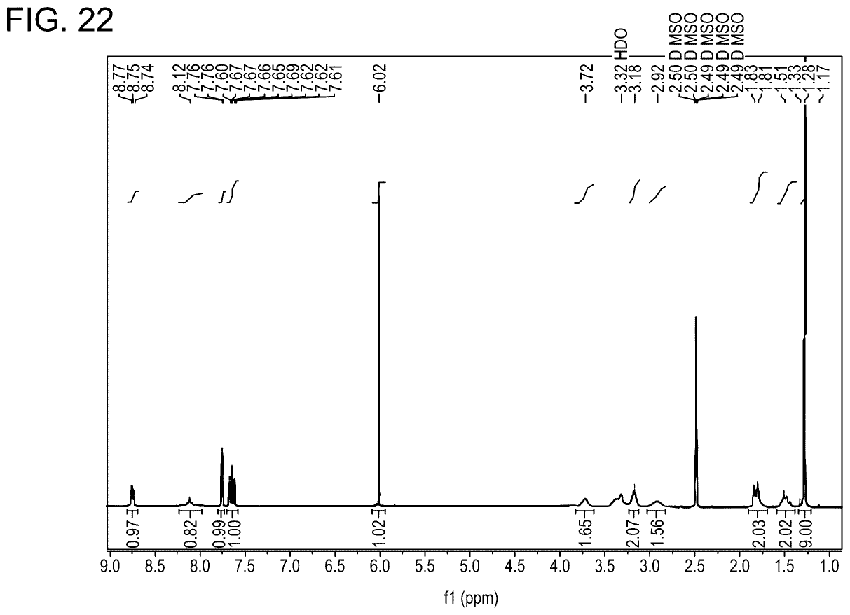

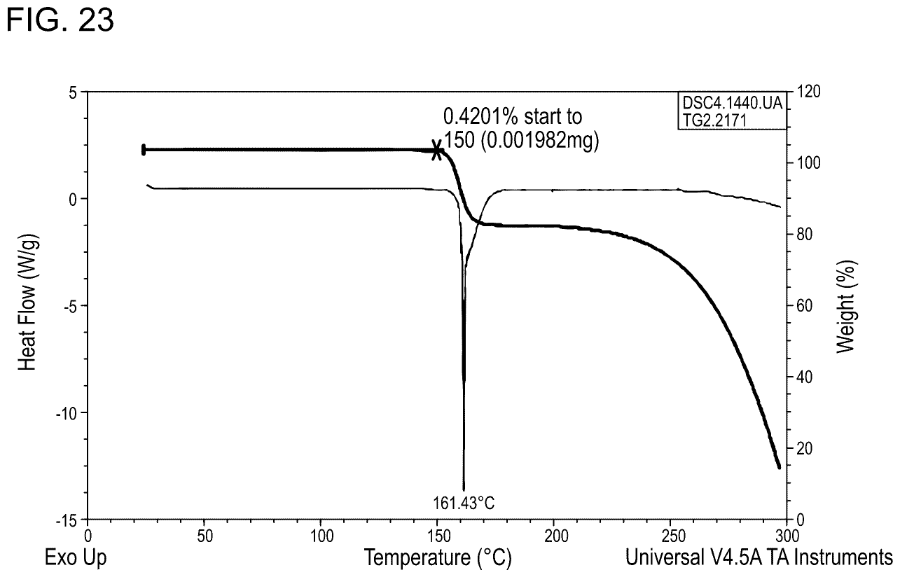

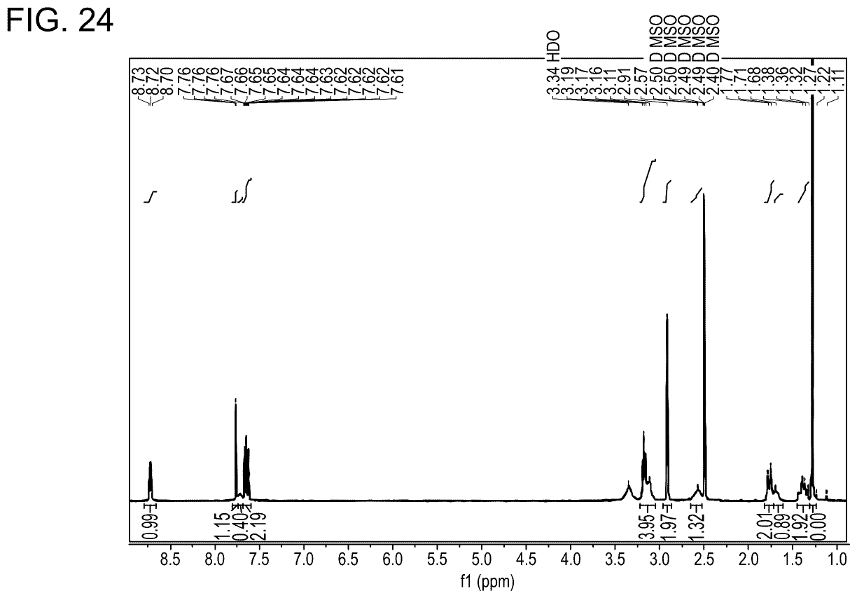

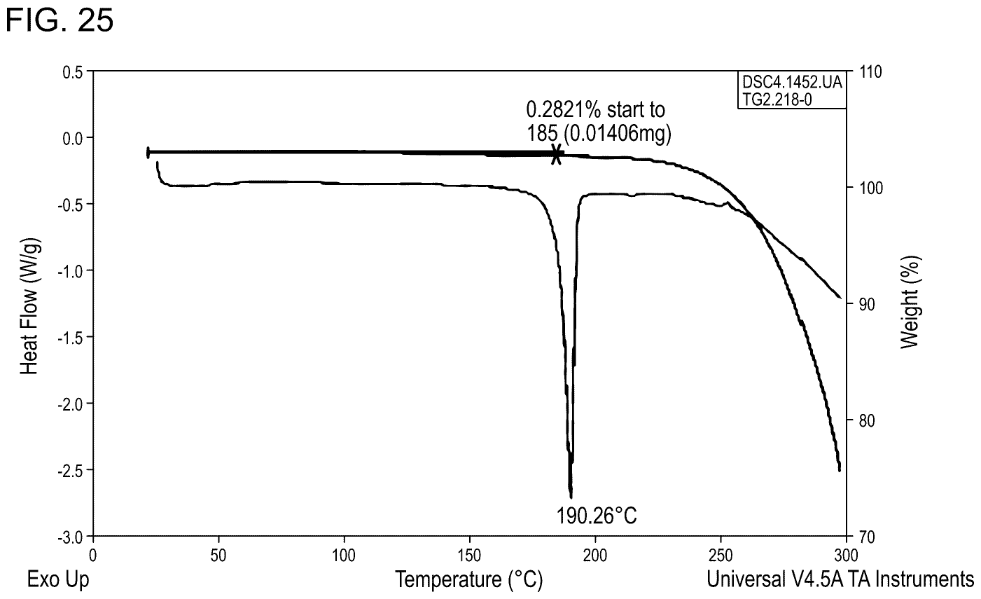

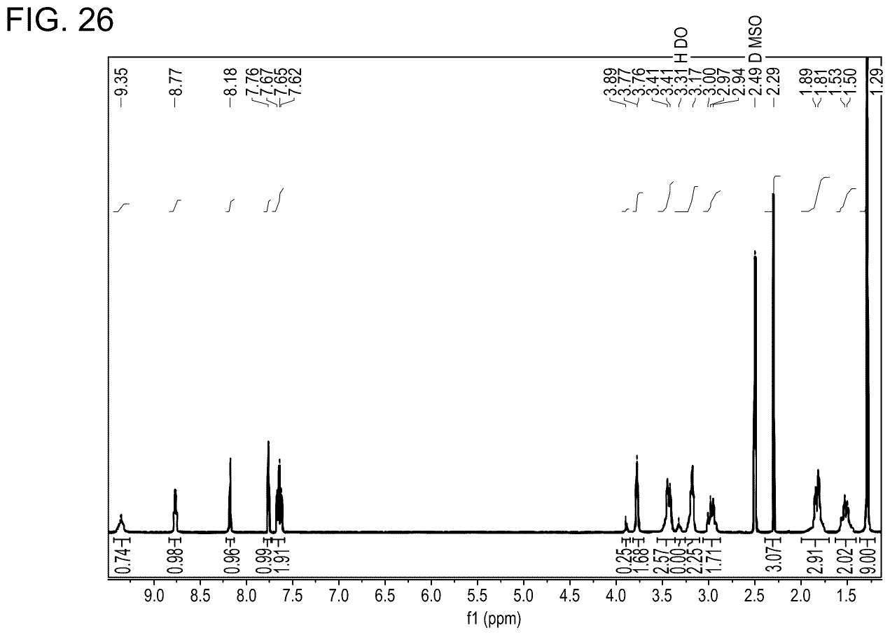

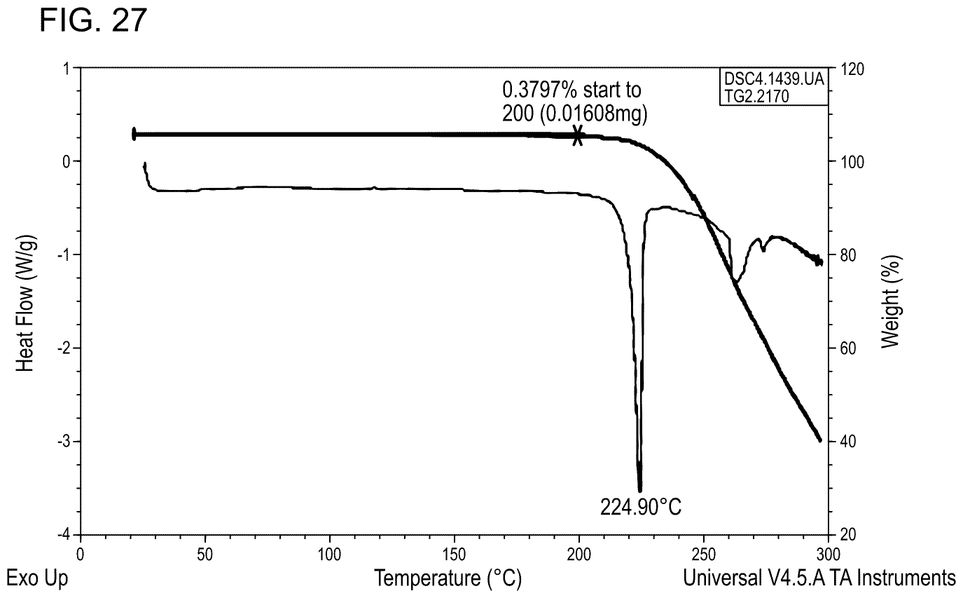

In some embodiments, crystalline Form 14 of fumarate salt of Compound 1 is characterized by an XRPD pattern substantially the same as the XRPD pattern depicted in FIG. 14. In some embodiments, the crystalline form of fumarate salt of Compound 1 is crystalline Form 14 characterized by an XRPD pattern comprising at least one peak, e.g., at least two peaks, at least three peaks, at least four peaks, or at least five or more peaks, at the following 2θ angles (±0.2°): 10.3°, 16.5°, 18.0°, 18.3°, 18.5°, 20.0°, 20.2°, 20.7°, 22.5°, 24.1°, 24.3°, 25.4°, and 26.5°. In another embodiment, the crystalline form is crystalline Form 14 characterized by an XRPD pattern comprising peaks at the following 2θ angles (±0.2°): 18.0°, 18.5°, 20.2°, and 22.5°.

In some embodiments, the crystalline Form 14 of fumarate salt of Compound 1 is at least about 60% a single crystalline form, at least about 70% a single crystalline form, at least about 80% a single crystalline form, at least about 90% a single crystalline form, at least about 95% a single crystalline form, or at least about 99% a single crystalline form by weight.

In some embodiments, the crystalline Form 14 of fumarate salt of Compound 1 is substantially free of amorphous forms of Compound 1.

### Crystalline Forms of Glycolate Salt of Compound 1

In some embodiments, the present disclosure provides crystalline forms of glycolate salt of Compound 1.

In some embodiments, the present disclosure provides crystalline Form 15 of glycolate salt of Compound 1 that is characterized by an XRPD pattern that comprises at least one peak, e.g., at least two peaks, at least three peaks, at least four peaks, or at least five or more peaks at the diffraction angle (° 2θ) selected from the group of peaks listed in Table 15.

TABLE 15
°2θ value for glycolate salt

| | | | Relative |
|---|---|---|---|
| Position | d-value | Height | Intensity |
| 7.3600 | 12.0108 | 998.0000 | 5.5779 |
| 8.2200 | 10.7560 | 1688.0000 | 9.4344 |
| 8.9000 | 9.9357 | 3222.0000 | 18.0080 |
| 10.3400 | 8.5550 | 3007.0000 | 16.8064 |
| 10.5600 | 8.3773 | 9390.0000 | 52.4816 |
| 10.7400 | 8.2373 | 1010.0000 | 5.6450 |
| 12.2400 | 7.2310 | 4972.0000 | 27.7890 |
| 12.5800 | 7.0363 | 1265.0000 | 7.0702 |
| 12.8200 | 6.9051 | 1220.0000 | 6.8187 |
| 13.2800 | 6.6669 | 2237.0000 | 12.5028 |
| 14.0000 | 6.3256 | 2086.0000 | 11.6588 |
| 14.2400 | 6.2196 | 1845.0000 | 10.3119 |
| 14.7000 | 6.0259 | 875.0000 | 4.8905 |
| 15.2200 | 5.8212 | 2370.0000 | 13.2461 |
| 15.4200 | 5.7462 | 1833.0000 | 10.2448 |
| 16.6200 | 5.3339 | 2402.0000 | 13.4250 |
| 16.8000 | 5.2771 | 3142.0000 | 17.5609 |
| 17.2600 | 5.1375 | 5181.0000 | 28.9571 |
| 17.5800 | 5.0447 | 4294.0000 | 23.9996 |
| 17.6800 | 5.0164 | 4675.0000 | 26.1290 |
| 17.6800 | 5.0164 | 4675.0000 | 26.1290 |
| 18.0800 | 4.9063 | 890.0000 | 4.9743 |
| 18.6000 | 4.7703 | 2386.0000 | 13.3356 |
| 19.2200 | 4.6178 | 7470.0000 | 41.7505 |
| 19.4200 | 4.5707 | 2985.0000 | 16.6834 |
| 19.9600 | 4.4482 | 1918.0000 | 10.7199 |
| 20.3800 | 4.3575 | 6304.0000 | 35.2336 |
| 20.6600 | 4.2991 | 17892.0000 | 100.0000 |
| 21.0000 | 4.2302 | 2787.0000 | 15.5768 |
| 21.5200 | 4.1292 | 3605.0000 | 20.1487 |
| 21.6800 | 4.0991 | 3611.0000 | 20.1822 |
| 21.9000 | 4.0584 | 1827.0000 | 10.2113 |
| 22.1200 | 4.0185 | 5187.0000 | 28.9906 |
| 22.4200 | 3.9654 | 5541.0000 | 30.9691 |
| 22.6400 | 3.9274 | 2096.0000 | 11.7147 |
| 22.9200 | 3.8800 | 4677.0000 | 26.1402 |
| 23.1000 | 3.8502 | 2955.0000 | 16.5158 |
| 23.3200 | 3.8144 | 5247.0000 | 29.3260 |
| 24.0400 | 3.7017 | 7510.0000 | 41.9741 |
| 25.6000 | 3.4796 | 2282.0000 | 12.7543 |
| 26.2200 | 3.3987 | 1112.0000 | 6.2151 |
| 26.7200 | 3.3362 | 4562.0000 | 25.4974 |
| 27.2600 | 3.2714 | 1620.0000 | 9.0543 |
| 27.7600 | 3.2136 | 1530.0000 | 8.5513 |
| 28.3200 | 3.1513 | 2987.0000 | 16.6946 |
| 28.6000 | 3.1211 | 1252.0000 | 6.9975 |
| 29.0400 | 3.0748 | 1252.0000 | 6.9975 |
| 29.3800 | 3.0400 | 986.0000 | 5.5108 |
| 29.8200 | 2.9961 | 1386.0000 | 7.7465 |
| 30.1200 | 2.9669 | 1238.0000 | 6.9193 |
| 30.3200 | 2.9478 | 1335.0000 | 7.4614 |
| 30.6200 | 2.9196 | 1298.0000 | 7.2546 |
| 31.1800 | 2.8684 | 2212.0000 | 12.3631 |
| 31.5400 | 2.8365 | 1034.0000 | 5.7791 |
| 32.4800 | 2.7565 | 1774.0000 | 9.9150 |
| 32.8000 | 2.7304 | 2269.0000 | 12.6816 |
| 33.1000 | 2.7063 | 1376.0000 | 7.6906 |
| 33.5000 | 2.6749 | 1248.0000 | 6.9752 |
| 34.1000 | 2.6292 | 1092.0000 | 6.1033 |
| 34.7000 | 2.5851 | 1162.0000 | 6.4945 |
| 35.0400 | 2.5608 | 902.0000 | 5.0414 |
| 35.5200 | 2.5273 | 1954.0000 | 10.9211 |
| 36.0200 | 2.4933 | 1110.0000 | 6.2039 |
| 36.5800 | 2.4565 | 815.0000 | 4.5551 |
| 37.1400 | 2.4207 | 1154.0000 | 6.4498 |
| 37.7200 | 2.3848 | 1606.0000 | 8.9761 |
| 38.1200 | 2.3607 | 1175.0000 | 6.5672 |
| 38.5600 | 2.3348 | 907.0000 | 5.0693 |

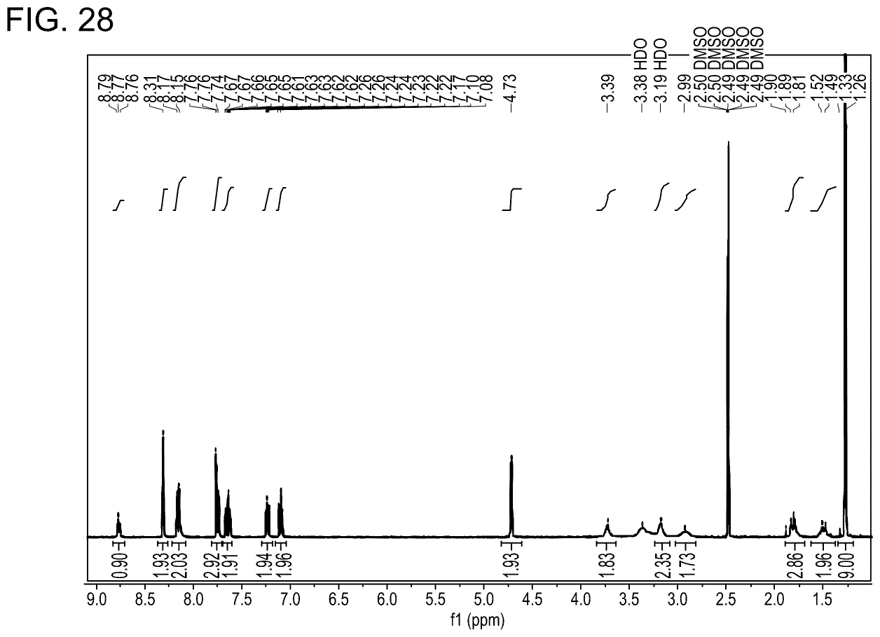

In some embodiments, crystalline Form 15 of glycolate salt of Compound 1 is characterized by an XRPD pattern substantially the same as the XRPD pattern depicted in FIG. 15. In some embodiments, the crystalline form of glycolate salt of Compound 1 is crystalline Form 15 characterized by an XRPD pattern comprising at least one peak, e.g., at least two peaks, at least three peaks, at least four peaks, or at least five or more peaks, at the following 2θ angles (±0.2°): 10.6°, 12.2°, 17.3°, 17.7°, 19.2°, 20.4°, 20.7°, 22.1°, 22.4°, 22.9°, 23.3°, and 24.0°. In another embodiment, the crystalline form of glycolate salt of Compound 1 is crystalline Form 15 characterized by an XRPD pattern comprising peaks at 2θ angles (±0.2°) 10.6°, 19.2°, 20.4°, 20.7°, and 24.0°.

In some embodiments, the crystalline Form 15 of glycolate salt of Compound 1 is at least about 60% a single crystalline form, at least about 70% a single crystalline form, at least about 80% a single crystalline form, at least about 90% a single crystalline form, at least about 95% a single crystalline form, or at least about 99% a single crystalline form by weight.

In some embodiments, the crystalline Form 15 of glycolate salt of Compound 1 is substantially free of amorphous forms of Compound 1.

### Crystalline Forms of Oxalate Salt of Compound 1

In some embodiments, the present disclosure provides crystalline forms of oxalate salt of Compound 1.

In some embodiments, the present disclosure provides crystalline Form 16 of oxalate salt of Compound 1 that is characterized by an XRPD pattern that comprises at least one peak, e.g., at least two peaks, at least three peaks, at least four peaks, or at least five or more peaks at the diffraction angle (° 2θ) selected from the group of peaks listed in Table 16.

TABLE 16
°2θ value for oxalate salt

| | | | Relative |
|---|---|---|---|
| Position | d-value | Height | Intensity |
| 5.6800 | 15.5589 | 43687.2109 | 100.0000 |
| 10.2000 | 8.6721 | 1521.4386 | 3.4826 |
| 10.5200 | 8.4090 | 4529.4526 | 10.3679 |
| 11.3400 | 7.8027 | 1529.5339 | 3.5011 |
| 11.8000 | 7.4996 | 1060.6533 | 2.4278 |
| 12.1800 | 7.2664 | 1414.2679 | 3.2373 |
| 12.9800 | 6.8203 | 2070.3398 | 4.7390 |
| 13.1000 | 6.7581 | 644.5694 | 1.4754 |
| 13.2200 | 6.6970 | 604.8115 | 1.3844 |
| 14.2600 | 6.2109 | 485.7940 | 1.1120 |
| 14.5400 | 6.0919 | 666.8890 | 1.5265 |
| 15.0000 | 5.9061 | 3785.4070 | 8.6648 |
| 16.1200 | 5.4982 | 691.0457 | 1.5818 |
| 16.3600 | 5.4181 | 627.7271 | 1.4369 |
| 16.5400 | 5.3595 | 621.0368 | 1.4216 |
| 17.0200 | 5.2094 | 12250.4053 | 28.0412 |
| 17.2200 | 5.1494 | 1798.3745 | 4.1165 |
| 17.5200 | 5.0619 | 1112.6129 | 2.5468 |
| 17.6400 | 5.0277 | 1697.6737 | 3.8860 |
| 18.0200 | 4.9225 | 902.4925 | 2.0658 |
| 18.2400 | 4.8636 | 3415.4158 | 7.8179 |
| 18.4600 | 4.8062 | 5973.9463 | 13.6744 |
| 18.6800 | 4.7501 | 3114.2297 | 7.1285 |
| 19.3200 | 4.5941 | 1214.2098 | 2.7793 |
| 19.7200 | 4.5018 | 1797.3568 | 4.1141 |
| 20.3400 | 4.3660 | 5857.4863 | 13.4078 |
| 20.5600 | 4.3198 | 5578.3921 | 12.7689 |
| 20.7400 | 4.2827 | 2111.5491 | 4.8333 |
| 21.1000 | 4.2104 | 2201.8508 | 5.0400 |
| 21.4600 | 4.1406 | 782.8640 | 1.7920 |
| 21.7000 | 4.0953 | 956.1747 | 2.1887 |
| 22.2000 | 4.0042 | 1701.2131 | 3.8941 |
| 22.3600 | 3.9759 | 1026.8710 | 2.3505 |
| 22.7400 | 3.9103 | 5771.1758 | 13.2102 |
| 22.9800 | 3.8700 | 3646.5190 | 8.3469 |
| 23.2400 | 3.8273 | 1542.3864 | 3.5305 |
| 23.6800 | 3.7572 | 1328.9153 | 3.0419 |
| 23.8600 | 3.7293 | 1421.9144 | 3.2548 |
| 24.0000 | 3.7078 | 2746.5574 | 6.2869 |
| 24.3600 | 3.6538 | 1957.2205 | 4.4801 |
| 24.7000 | 3.6043 | 2356.8975 | 5.3949 |
| 24.8000 | 3.5900 | 6920.2871 | 15.8405 |
| 25.1800 | 3.5367 | 1165.9471 | 2.6689 |
| 25.5200 | 3.4903 | 1234.0406 | 2.8247 |
| 25.6600 | 3.4716 | 3628.8020 | 8.3063 |
| 26.1000 | 3.4141 | 533.1056 | 1.2203 |
| 26.6200 | 3.3485 | 834.6998 | 1.9106 |
| 27.0600 | 3.2951 | 603.6350 | 1.3817 |
| 27.2200 | 3.2761 | 1740.1896 | 3.9833 |
| 28.1400 | 3.1710 | 1700.5569 | 3.8926 |
| 28.5600 | 3.1253 | 487.5840 | 1.1161 |
| 29.2800 | 3.0501 | 656.9351 | 1.5037 |
| 29.7200 | 3.0059 | 486.1585 | 1.1128 |
| 30.1400 | 2.9650 | 729.5934 | 1.6700 |
| 30.5400 | 2.9271 | 519.8840 | 1.1900 |
| 30.9400 | 2.8901 | 825.3423 | 1.8892 |
| 31.3400 | 2.8542 | 616.9506 | 1.4122 |
| 31.8200 | 2.8122 | 811.1391 | 1.8567 |
| 32.2600 | 2.7748 | 745.9998 | 1.7076 |
| 32.5400 | 2.7516 | 913.6664 | 2.0914 |
| 32.7600 | 2.7336 | 613.0424 | 1.4033 |
| 33.0000 | 2.7143 | 803.0989 | 1.8383 |
| 33.2800 | 2.6921 | 503.6571 | 1.1529 |
| 33.9200 | 2.6427 | 463.1584 | 1.0602 |
| 34.0600 | 2.6322 | 457.7043 | 1.0477 |
| 34.3800 | 2.6084 | 1676.8276 | 3.8383 |
| 34.4800 | 2.6011 | 892.6708 | 2.0433 |
| 34.6600 | 2.5880 | 612.5185 | 1.4021 |
| 34.8600 | 2.5736 | 478.3242 | 1.0949 |
| 35.1400 | 2.5537 | 529.7245 | 1.2125 |
| 35.4800 | 2.5300 | 529.7609 | 1.2126 |
| 35.8200 | 2.5068 | 664.4661 | 1.5210 |
| 35.9400 | 2.4987 | 943.9102 | 2.1606 |
| 36.9000 | 2.4359 | 690.8985 | 1.5815 |
| 37.8200 | 2.3787 | 591.3420 | 1.3536 |
| 39.2800 | 2.2936 | 516.9450 | 1.1833 |

In some embodiments, crystalline Form 16 of oxalate salt of Compound 1 is characterized by an XRPD pattern substantially the same as the XRPD pattern depicted in FIG. 16. In some embodiments, the crystalline form of oxalate salt of Compound 1 is crystalline Form 16 characterized by an XRPD pattern comprising at least one peak, e.g., at least two peaks, at least three peaks, at least four peaks, or at least five or more peaks, at the following 2θ angles (±0.2°): 5.7°, 10.5°, 17.0°, 18.5°, 20.3°, 20.6°, 22.7°, and 24.8°. In another embodiment, the crystalline form of oxalate salt of Compound 1 is crystalline Form 16 characterized an XRPD pattern comprising peaks at 2θ angles (±0.2°) 5.7°, 17.0°, and 24.8°.

In some embodiments, the crystalline Form 16 of oxalate salt of Compound 1 is at least about 60% a single crystalline form, at least about 70% a single crystalline form, at least about 80% a single crystalline form, at least about 90% a single crystalline form, at least about 95% a single crystalline form, or at least about 99% a single crystalline form by weight.

In some embodiments, the crystalline Form 16 of oxalate salt of Compound 1 is substantially free of amorphous forms of Compound 1.

### Crystalline Forms of Phosphate Salt of Compound 1

In some embodiments, the present disclosure provides crystalline forms of phosphate salt of Compound 1.

In some embodiments, the present disclosure provides crystalline Form 17 of phosphate salt of Compound 1 that is characterized by an XRPD pattern that comprises at least one peak, e.g., at least two peaks, at least three peaks, at least four peaks, or at least five or more peaks at the diffraction angle (° 2θ) selected from the group of peaks listed in Table 17.

TABLE 17
°2θ value for phosphate salt

| | | | Relative |
|---|---|---|---|
| Position | d-value | Height | Intensity |
| 5.7200 | 15.4502 | 2936.0000 | 29.6506 |
| 7.0600 | 12.5205 | 2528.0000 | 25.5302 |
| 10.2600 | 8.6215 | 1672.0000 | 16.8855 |
| 10.5200 | 8.4090 | 854.0000 | 8.6245 |
| 10.8800 | 8.1316 | 1233.0000 | 12.4520 |
| 11.3600 | 7.7890 | 1865.0000 | 18.8346 |
| 13.4200 | 6.5977 | 1271.0000 | 12.8358 |
| 14.0400 | 6.3077 | 2179.0000 | 22.0057 |
| 15.1400 | 5.8518 | 1890.0000 | 19.0871 |
| 15.8200 | 5.6018 | 945.0000 | 9.5435 |
| 16.3600 | 5.4181 | 1270.0000 | 12.8257 |
| 17.0200 | 5.2094 | 1278.0000 | 12.9065 |
| 18.0600 | 4.9117 | 5741.0000 | 57.9782 |
| 18.8200 | 4.7150 | 9902.0000 | 100.0000 |
| 19.0400 | 4.6611 | 4901.0000 | 49.4951 |
| 20.1600 | 4.4046 | 1901.0000 | 19.1981 |
| 20.5000 | 4.3323 | 4309.0000 | 43.5165 |
| 21.1200 | 4.2065 | 7940.0000 | 80.1858 |
| 21.7600 | 4.0842 | 5066.0000 | 51.1614 |
| 22.4800 | 3.9550 | 1867.0000 | 18.8548 |
| 22.8800 | 3.8867 | 4365.0000 | 44.0820 |
| 23.0600 | 3.8568 | 3817.0000 | 38.5478 |
| 25.0800 | 3.5506 | 1702.0000 | 17.1884 |
| 25.3200 | 3.5174 | 2651.0000 | 26.7724 |
| 26.2200 | 3.3987 | 2811.0000 | 28.3882 |
| 26.5800 | 3.3535 | 1468.0000 | 14.8253 |
| 26.6600 | 3.3436 | 1415.0000 | 14.2900 |
| 26.8000 | 3.3265 | 1335.0000 | 13.4821 |
| 26.9400 | 3.3095 | 1546.0000 | 15.6130 |
| 27.3600 | 3.2596 | 1238.0000 | 12.5025 |
| 28.2400 | 3.1600 | 1878.0000 | 18.9659 |
| 28.5200 | 3.1296 | 1330.0000 | 13.4316 |
| 28.8400 | 3.0956 | 1187.0000 | 11.9875 |
| 29.5800 | 3.0199 | 2150.0000 | 21.7128 |
| 30.0000 | 2.9785 | 1089.0000 | 10.9978 |
| 30.5000 | 2.9308 | 1573.0000 | 15.8857 |
| 31.3400 | 2.8542 | 1943.0000 | 19.6223 |
| 31.9200 | 2.8036 | 1333.0000 | 13.4619 |
| 32.4200 | 2.7615 | 923.0000 | 9.3213 |
| 32.8000 | 2.7304 | 1006.0000 | 10.1596 |
| 33.8800 | 2.6458 | 1065.0000 | 10.7554 |
| 35.9000 | 2.5014 | 1033.0000 | 10.4322 |
| 37.1400 | 2.4207 | 1206.0000 | 12.1794 |
| 37.4600 | 2.4007 | 958.0000 | 9.6748 |
| 38.0800 | 2.3631 | 1192.0000 | 12.0380 |
| 39.1000 | 2.3037 | 999.0000 | 10.0889 |
| 39.7200 | 2.2692 | 1416.0000 | 14.3001 |

In some embodiments, crystalline Form 17 of phosphate salt of Compound 1 is characterized by an XRPD pattern substantially the same as the XRPD pattern depicted in FIG. 17. In some embodiments, the crystalline form of phospates salt of Compound 1 is crystalline Form 17 characterized by an XRPD pattern comprising at least one peak, e.g., at least two peaks, at least three peaks, at least four peaks, or at least five or more peaks, at the following 2θ angles (±0.2°): 5.7°, 7.1°, 18.1°, 18.8°, 19.0°, 20.5°, 21.2°, 21.8°, 22.9°, 23.1°, 25.3°, and 26.2°. In another embodiment, the crystalline form of phosphate salt of Compound 1 is crystalline Form 17 characterized by an XRPD pattern comprising peaks at the following 2θ angles (±0.2°): 18.1°, 18.8°, 21.2°, and 21.8°.

In some embodiments, the crystalline Form 17 of phosphate salt of Compound 1 is at least about 60% a single crystalline form, at least about 70% a single crystalline form, at least about 80% a single crystalline form, at least about 90% a single crystalline form, at least about 95% a single crystalline form, or at least about 99% a single crystalline form by weight.

In some embodiments, the crystalline Form 17 of phosphate salt of Compound 1 is substantially free of amorphous forms of Compound 1.

### Crystalline Forms of Sebacate Salt of Compound 1

In some embodiments, the present disclosure provides crystalline forms of sebacate salt of Compound 1.

In some embodiments, the present disclosure provides crystalline Form 18 of sebacate salt of Compound 1 that is characterized by an XRPD pattern that comprises at least one peak, e.g., at least two peaks, at least three peaks, at least four peaks, or at least five or more peaks at the diffraction angle (° 2θ) selected from the group of peaks listed in Table 18.

TABLE 18
°2θ value for sebacate salt

| | | | Relative |
|---|---|---|---|
| Position | d-value | Height | Intensity |
| 8.2200 | 10.7560 | 667.0406 | 40.7297 |
| 11.8600 | 7.4618 | 812.1450 | 49.5898 |
| 13.2400 | 6.6870 | 782.8215 | 47.7993 |
| 14.0800 | 6.2899 | 353.2725 | 21.5709 |
| 14.7400 | 6.0097 | 404.3534 | 24.6899 |
| 16.1400 | 5.4914 | 422.7616 | 25.8140 |
| 16.4200 | 5.3984 | 1453.0674 | 88.7247 |
| 17.1000 | 5.1852 | 490.0565 | 29.9230 |
| 17.9400 | 4.9443 | 572.6228 | 34.9645 |
| 18.5000 | 4.7959 | 541.4030 | 33.0582 |
| 19.0000 | 4.6708 | 559.0601 | 34.1364 |
| 19.6200 | 4.5246 | 766.8754 | 46.8256 |
| 19.8600 | 4.4704 | 869.2078 | 53.0741 |
| 20.4600 | 4.3407 | 1637.7251 | 100.0000 |
| 21.3000 | 4.1713 | 690.7946 | 42.1801 |
| 21.6200 | 4.1103 | 1163.2998 | 71.0314 |
| 22.0600 | 4.0293 | 1236.2267 | 75.4844 |
| 23.0000 | 3.8667 | 484.0168 | 29.5542 |
| 23.7600 | 3.7447 | 530.4952 | 32.3922 |
| 24.2200 | 3.6746 | 777.2438 | 47.4587 |
| 24.6400 | 3.6129 | 546.8872 | 33.3931 |
| 24.7800 | 3.5929 | 465.8834 | 28.4470 |
| 25.4200 | 3.5038 | 507.5801 | 30.9930 |
| 26.1000 | 3.4141 | 345.9577 | 21.1243 |
| 26.5600 | 3.3560 | 528.5072 | 32.2708 |
| 26.9800 | 3.3047 | 524.4874 | 32.0254 |
| 27.3000 | 3.2667 | 366.0664 | 22.3521 |
| 28.0600 | 3.1799 | 417.9956 | 25.5229 |
| 28.3600 | 3.1469 | 492.7919 | 30.0900 |
| 29.5200 | 3.0259 | 484.3113 | 29.5722 |
| 30.2400 | 2.9554 | 506.6928 | 30.9388 |
| 31.6000 | 2.8313 | 431.4577 | 26.3449 |
| 32.3600 | 2.7665 | 316.6806 | 19.3366 |
| 33.0000 | 2.7143 | 331.5425 | 20.2441 |
| 33.6800 | 2.6610 | 306.4579 | 18.7124 |
| 34.2000 | 2.6217 | 290.1310 | 17.7155 |
| 34.5800 | 2.5938 | 299.0270 | 18.2587 |
| 35.2400 | 2.5467 | 277.1468 | 16.9227 |
| 35.9200 | 2.5001 | 304.9290 | 18.6191 |
| 36.4000 | 2.4682 | 306.1805 | 18.6955 |
| 37.2400 | 2.4144 | 331.3577 | 20.2328 |
| 38.6400 | 2.3301 | 350.1133 | 21.3780 |
| 39.9600 | 2.2561 | 613.0710 | 37.4343 |

In some embodiments, crystalline Form 18 of sebacate salt of Compound 1 is characterized by an XRPD pattern substantially the same as the XRPD pattern depicted in FIG. 18. In some embodiments, the crystalline form of sebacate salt of Compound 1 is crystalline Form 18 characterized by an XRPD pattern comprising at least one peak, e.g., at least two peaks, at least three peaks, at least four peaks, or at least five or more peaks, at the following 2θ angles (±0.2°): 8.2°, 11.9°, 16.4°, 19.6°, 19.8°, 20.5°, 21.3°, 21.6°, 22.1°, and 24.2°. In another embodiment, the crystalline form of sebacate salt of Compound 1 is crystalline Form 18 characterized by an XRPD pattern comprising peaks at 2θ angles (±0.2°) 16.4°, 19.8°, 20.5°, 21.6°, and 22.1°.

In some embodiments, the crystalline Form 18 of sebacate salt of Compound 1 is at least about 60% a single crystalline form, at least about 70% a single crystalline form, at least about 80% a single crystalline form, at least about 90% a single crystalline form, at least about 95% a single crystalline form, or at least about 99% a single crystalline form by weight.

In some embodiments, the crystalline Form 18 of sebacate salt of Compound 1 is substantially free of amorphous forms of Compound 1.

### Compositions Comprising Salts and Crystalline Forms of Compound 1

The present disclosure also provides pharmaceutical compositions comprising the salts and crystalline salt forms of Compound 1 provided by the present disclosure. The amount of a salt or a crystalline salt form of Compound 1 in a provided composition is such that Compound 1 can act as an effective T-type calcium channel blocker. For example, the amount of a salt or a crystalline salt form of Compound 1 in a pharmaceutical composition is such that it is effective for treating, e.g., a neurological disorder, a psychiatric disorder, a generalized epileptic syndrome, or a tremor, including an essential tremor.

Pharmaceutical compositions described herein can be prepared by any method known in the art of pharmacology. In general, such preparatory methods include the steps of bringing a salt or a crystalline salt form of Compound 1 provided by the present disclosure into association with a carrier and/or one or more other accessory ingredients, and then, if necessary and/or desirable, shaping and/or packaging the product into a desired single- or multi-dose unit.

Pharmaceutically acceptable carriers used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Carriers such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween 20), polyoxyethylene sorbitan (Tween 60), polyoxyethylene sorbitan monooleate (Tween 80), sorbitan monopalmitate (Span 40), sorbitan monostearate (Span 60), sorbitan tristearate (Span 65), glyceryl monooleate, sorbitan monooleate (Span 80)), polyoxyethylene esters (e.g., polyoxyethylene monostearate (Myrj 45), polyoxyethylene hydrogenated castor oil, polyethoxylated castor oil, polyoxymethylene stearate, and Solutol), sucrose fatty acid esters, polyethylene glycol fatty acid esters (e.g., Cremophor™), polyoxyethylene ethers, (e.g., polyoxyethylene lauryl ether (Brij 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F-68, Poloxamer-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Ncolonc, Kathon, and Euxyl.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethaminc, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, sodium stearyl fumarate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

The pharmaceutical compositions provided herein may be administered orally, parenterally, by inhalation spray, topically, rectally, nasally, buccally, transmucosally, or in an ophthalmic preparation. The term “parenteral” as used herein includes subcutaneous, intravenous, intramuscular, intra-articular, intra-synovial, intrasternal, intrathecal, intrahepatic, intralesional and intracranial injection or infusion techniques. In one aspect, the pharmaceutical compositions provided herein are orally administered in an orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

The amount of a salt or a crystalline salt form of Compound 1 to be combined with carrier materials to produce a composition in a single dosage form will vary depending upon the subject to be treated and the mode of administration. For example, a specific dosage and treatment regimen for a subject will depend upon a variety of factors, including age, body weight, general health, sex, diet, time of administration, rate of excretion, drug combination, the judgment of the treating physician, and the severity of the particular disease being treated. In one aspect, a pharmaceutical composition may be formulated such that a dosage equivalent to about 0.001 to about 100 mg/kg body weight/day of a salt or a crystalline salt form of Compound 1 (e.g., about 0.5 to about 100 mg/kg of Compound 1) can be administered to a subject receiving these compositions. Alternatively, dosages equivalent to 1 mg/kg and 1000 mg/kg of compound 1 every 4 to 120 hours are also acceptable. As used herein, the dose refers to the amount of a salt or a crystalline salt form of Compound 1 provided by the present disclosure.

In some embodiments, a salt or a crystalline salt form of Compound 1 provided by the present disclosure is formulated for administration at a dose equivalent to about 2 mg to about 3000 mg of Compound 1, e.g., about 5 mg to about 350 mg, about 5 mg to about 200 mg, about 5 mg to about 100 mg, about 20 mg to about 40 mg of Compound 1. In certain embodiments, a salt or a crystalline salt form of Compound 1 is formulated for administration at a dose equivalent to about 5 mg, about 10 mg, about 15 mg, about 20 mg, about 30 mg, about 40 mg, about 45 mg, about 50 mg, about 60 mg, about 70 mg, about 80 mg, about 90 mg, about 100 mg, about 110 mg or about 120 mg of Compound 1.

In some embodiments, a salt or a crystalline salt form of Compound 1 provided by the present disclosure is formulated for administration at a dose equivalent to about 2 mg to about 3000 mg of Compound 1 per day, e.g., about 5 mg to about 500 mg, about 5 mg to about 200 mg, about 20 mg to about 40 mg of Compound 1 per day. In some embodiments, a salt or a crystalline salt form of Compound 1 provided by the present disclosure is formulated for administration at a dose equivalent to about 5 mg, about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, or about 150 mg of Compound 1 per day. In some embodiments, a salt or a crystalline salt form of Compound 1 provided by the present disclosure may be administered once per day. In other embodiments, a salt or a crystalline salt form of Compound 1 provided by the present disclosure may be administered twice per day. In yet other embodiments, a salt or a crystalline salt form of Compound 1 provided by the present disclosure may be administered once every other day.

In some embodiments, methods of the present disclosure comprise administering to a subject in need thereof a titrated dose of a salt or a crystalline form of Compound 1 provided by the present disclosure. In some embodiments, the maximum titrated dose is 60 mg per day or 100 mg per day. In one embodiment, the maximum titrated dose is 60 mg per day.

In some embodiments, a salt or a crystalline salt form of Compound 1 provided by the present disclosure may be formulated as a tablet composition together with a pharmaceutically acceptable carrier. Suitable carriers that may be used in a tablet composition may be selected from one or more of microcrystalline cellulose, mannitol, Croscarmellose Sodium, and Sodium Stearyl Fumarate. For example, in some embodiments, the carrier may be microcrystalline cellulose, e.g., present in the tablet composition in an amount of about 40% w/w to about 80% w/w, e.g., about 50% w/w to about 70% w/w, or about 55% w/w to about 65% w/w. In some embodiments, the carrier may be mannitol, e.g., present in the tablet composition in an amount of 20% w/w to about 45% w/w, e.g., about 20% w/w to about 30% w/w. In some embodiments, the carrier may be croscarmellose sodium, e.g., present in an amount of about 1% w/w to about 5% w/w. In some embodiments, the carrier may be stearyl fumarate, e.g., present in in the tablet composition an amount of about 1% w/w to about 5% w/w.

In some embodiments, a salt or a crystalline salt form of Compound 1 provided by the present disclosure may be present in the tablet composition in an amount equivalent to about 1 mg to about 150 mg of Compound 1, e.g., about 1 mg to about 100 mg, about 5 mg to about 50 mg or about 20 mg to about 40 mg of Compound 1.

In one aspect, the pharmaceutical compositions provided by the present disclosure include a single-unit dosage form comprising a salt or a crystalline salt form of Compound 1. In certain embodiments, single-unit dosage form comprises up to 200 mg of a salt or a crystalline salt form of Compound 1. In some embodiments, the single-unit dosage form comprises a length of up to 16 mm, e.g., about 14 mm to about 16 mm, and/or a width of up to 7 mm, e.g., about 5 mm to about 7 mm. In some embodiments, the single-unit dosage form is bioequivalent to a reference composition of the same dosage strength administered as multiple dosage forms, such as the a modified release (MR) formulation comprising compound 1 available as 5-mg and 20-mg tablets that are round and small in size (e.g., about 6 mm in diameter). In some embodiments, bioequivalency may be established by: (a) a 90% Confidence Interval for AUC which is between about 80% and about 125%, and (b) a 90% Confidence Interval for Cmax, which is between about 80% and about 125%.

In some embodiments, methods of the present disclosure comprise administering to a subject in need thereof a single-unit dosage form, comprising a salt or a crystalline salt form of Compound 1, wherein the composition is bioequivalent to a reference composition of the same dosage strength administered as multiple smaller, round tablets.

In some embodiments, a pharmaceutical composition provided by the present disclosure may be administered to the subject once per day or more than once a day (e.g., twice a day, three times a day, or four times a day).

### Modified-Release Dosage Forms and Compositions

In some embodiments, the present disclosure also provides a pharmaceutical composition comprising a salt or a crystalline salt form of Compound land an excipient that functions to modify the release rate of the Compound 1. In some embodiments, the pharmaceutical composition may be a swellable core technology formulation.

A composition of the present disclosure may comprise a salt or a crystalline salt form of Compound 1 and a modified-release polymer. The term “modified-release polymer”, as used herein, refers to any polymer that, when added to a composition comprising a salt or a crystalline salt form of Compound 1, modifies the release rate of Compound 1. A modified-released polymer may be a hydrophilic polymer, e.g., a hydroxypropyl methyl cellulose (HPMC) polymer; a hydrophobic polymer, e.g., ethyl cellulose or ethocell; or a polyacrylate polymer, e.g., Eudragit RL100, Eudragit RS100. In some embodiments, the modified-release polymer is present in the composition in an amount sufficient to modify the release rate of Compound 1. In some embodiments, the composition is for oral administration.

In some embodiments, a composition of the disclosure may comprise a modified-release polymer and a salt or a crystalline salt form of Compound 1 in an amount of from about 0.9% by weight to about 40% by weight (e.g., from about 0.9% by weight to about 30%, from about 1% by weight to about 25% by weight, from about 2% by weight to about 25% by weight, from about 3% by weight to about 20% by weight, from about 4% by weight to about 20% by weight, from about 5% by weight to about 20% by weight, from about 5% by weight to about 15% by weight, from about 5% by weight to about 10% by weight, or about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 40% by weight.

In some embodiments, a composition of the disclosure may comprise a modified-release polymer and a salt or a crystalline salt form of Compound 1 in an amount of from about 1 mg to about 40 mg, e.g., about 5 mg, about 10 mg, about 15 mg, about 20 mg, about 25 mg, about 30 mg, about 35 mg, about 40 mg.

In some embodiments, the composition comprises from about 55 mg to about 65 mg of a modified-release polymer (e.g., an HPMC polymer). In some embodiments, the composition comprises from about 10% by weight to about 70% by weight, e.g., from about 50% by weight to about 60% by weight of the modified-release polymer (e.g., an HPMC polymer).

In some embodiments, the composition may further comprise a diluent, e.g., microcrystalline cellulose. In some embodiments, the composition may comprise microcrystalline cellulose in an amount from about 15 mg to 40 mg, e.g., from about 15 mg to about 25 mg, from about 20 mg to about 25 mg, from about 25 mg to about 30 mg, or from about 30 mg to about 40 mg. In some embodiments, the composition may comprise microcrystalline cellulose in an amount of from about 15% to about 35% by weight, e.g., from about 15% to about 20%, from about 20% to about 25%, from 25% to about 30%, or from 30% to about 35% by weight.

In some embodiments, the composition may further comprise a glidant, e.g., colloidal silicon dioxide. In some embodiments, the composition may further comprise a lubricant, e.g., magnesium stearate. In some embodiments, the composition may further comprise a coating.

In some embodiments, the composition may be administered to a patient once daily or twice daily. In some embodiments, the composition may be in a form of a tablet, a capsule or a suspension. In one embodiment, the composition is in a form of a tablet.

### Methods of Treatment Using a Salt or a Crystalline Salt Form of Compound 1

The present disclosure also provides methods of treating a disease, disorder or condition relating to function of T-type calcium channel. The method comprises administering to a subject in need thereof an effective amount of a salt or a crystalline salt form of Compound 1, or a pharmaceutical composition comprising a salt or a crystalline salt form of Compound 1 described herein. The ability of Compound 1 to modulate T-type Calcium Channels has been described, e.g., in at least, WO 2021/007487, the entire contents of which are hereby incorporated herein by reference.

In some embodiments, the present disclosure provides a method of treating a neurological disorder in a subject in need thereof, the method comprising administering to the subject an effective amount of a salt or a crystalline salt form of Compound 1, or a pharmaceutical composition comprising a salt of a crystalline salt form of Compound 1 described herein. In some embodiments, the subject has a mutation in one or both of the T-type calcium channel genes CACNA1H and CACNA1G. In some embodiments, the neurological disorder is epilepsy. In some embodiments, the epilepsy is juvenile epilepsy. In some embodiments, the epilepsy is genetic epilepsy. In some embodiments, the neurological disorder is absence seizure. In some embodiments, the neurological disorder is pain (e.g., acute pain, chronic pain, neuropathic pain, inflammatory pain, nociceptive pain, central pain, thalamic pain or migraine. In some embodiments, the neurological disorder is tremor (e.g., essential tremor, or a Parkinsonian tremor). In some embodiments, the neurological disorder is ataxia (e.g., spinocerebellar ataxia, or spinocerebellar ataxia with CACNA1G mutations). In some embodiments, the neurological disorder is tinnitus. In certain embodiments, the neurological disorder is a disorder of wakefulness.

In some embodiments, a salt or a crystalline salt form of Compound 1, or a pharmaceutical composition comprising a salt or a crystalline salt form of Compound 1 described herein, is effective in the treatment of tremor (e.g., essential tremor). In some embodiments, a salt or a crystalline salt form of Compound 1, or a pharmaceutical composition comprising a salt or a crystalline salt form of Compound 1 described herein, is effective in the treatment of an epilepsy or epilepsy syndrome, e.g., absence seizures, juvenile myoclonic epilepsy, status epilepticus, or a genetic epilepsy. A salt or a crystalline salt form of Compound 1, or a pharmaceutical composition comprising a salt or a crystalline salt form of Compound 1 described herein may also modulate all T-type calcium channels, e.g., Cav3.1, Cav3.2, and/or Cav3.3. In some embodiments, a salt or a crystalline salt form of Compound 1, or a pharmaceutical composition comprising a salt or a crystalline salt form of Compound 1 described herein, is effective in the treatment of a psychiatric disorder, e.g., mood disorder, e.g., major depressive disorder.

In some embodiments, the present disclosure also provides a method of treating a psychiatric disorder in a subject in need thereof, the method comprising administering to the subject an effective amount of a salt or a crystalline salt form of Compound 1 or a pharmaceutical composition comprising a salt or a crystalline salt form of Compound 1 described herein. In some embodiments, the psychiatric disorder is a mood disorder. In some embodiments, the mood disorder is major depressive disorder.

### Epilepsy and Epilepsy Syndromes

A salt or a crystalline salt form of Compound 1 or a pharmaceutical composition comprising a salt or a crystalline salt form of Compound 1 described herein may be useful in the treatment of epilepsy and epilepsy syndromes. Epilepsy is a CNS disorder in which nerve cell activity in the brain becomes disrupted, causing seizures which can manifest as abnormal movements, periods of unusual behavior, sensations and sometimes loss of consciousness. Seizure symptoms will vary widely, from a simple blank stare for a few seconds to repeated twitching of their arms or legs during a seizure.

Epilepsy may involve a generalized seizure or a partial or focal seizure. All areas of the brain are involved in a generalized seizure. A person experiencing a generalized seizure may cry out or make some sound, stiffen for several seconds to a minute and then have rhythmic movements of the arms and legs. The eyes are generally open, the person may appear not to be breathing and actually turn blue. The return to consciousness is gradual and the person maybe confused from minutes to hours. The following are the main types of generalized seizures: tonic-clonic, tonic, clonic, myoclonic, myoclonic-tonic-clonic, myoclonic-atonic, atonic, and absence (typical, atypical, myoclonic, eyelid myoclonia) seizures, and epileptic spasms. In a partial or focal seizure, only part of the brain is involved, so only part of the body is affected. Depending on the part of the brain having abnormal electrical activity, symptoms may vary.

Epilepsy, as described herein, includes a generalized, partial, complex partial (e.g., seizures involving only part of the brain, but where consciousness is compromised), tonic clonic, clonic, tonic, refractory seizures, status epilepticus, absence seizures, febrile seizures, or temporal lobe epilepsy.

A salt or a crystalline salt form of Compound 1 or a pharmaceutical composition comprising a salt or a crystalline salt form of Compound 1 described herein may also be useful in the treatment of epilepsy syndromes. Severe syndromes with diffuse brain dysfunction caused, at least partly, by some aspect of epilepsy, are also referred to as epileptic encephalopathies. These are associated with frequent seizures that are resistant to treatment and severe cognitive dysfunction, for instance West syndrome.

In some embodiments, the epilepsy syndrome comprises epileptic encephalopathy, Dravet syndrome, Angelman syndrome, CDKL5 disorder, frontal lobe epilepsy, infantile spasms, West's syndrome, Juvenile Myoclonic Epilepsy, Landau-Kleffner syndrome, Lennox-Gastaut syndrome, Ohtahara syndrome, PCDH19 epilepsy, or Glut1 deficiency. In some embodiments, the epilepsy syndrome is childhood absence epilepsy (CAE). In some embodiments, the epilepsy syndrome is juvenile absence epilepsy (JAE). In some embodiments, the epilepsy syndrome is Lennox-Gastaut syndrome. In some embodiments, the epilepsy syndrome is SLC6A1 epileptic encephalopathy. In some embodiments, the epilepsy syndrome is associated with mutations in the genes that code for T-type calcium channels (e.g., CACNA1G, EEF1A2, and GABRG2 for genetic generalized epilepsy (GGE) and LGI1, TRIM3, and GABRG2 for non-acquired focal epilepsy (NAFE)). In some embodiments, the epilepsy syndrome is Doose syndrome or myoclonic astatic epilepsy. In some embodiments, the epilepsy syndrome is epileptic encephalopathy with continuous spike and wave during sleep (CSWS). In some embodiments, the epilepsy syndrome is Landau Kleffner Syndrome (LKS). In some embodiments, the epilepsy syndrome is Jeavons syndrome.

### Absence Seizures

Absence seizures are one of the most common seizure types in patients with idiopathic generalised epilepsy (IGE). Absence seizures are relatively brief, non-convulsive seizures characterised by abrupt onset of loss of awareness and responsiveness, usually lasting between 10-30 seconds in duration, with a rapid return to normal consciousness without post-ictal confusion. The seizures are characterised on an accompanying EEG recording by the abrupt onset and offset of generalised 1-6 Hz (e.g., 3 Hz) spike and wave discharges. Absence seizure often occur multiple times per day, interrupt learning and psychosocial functioning, and present a risk of injury because of the frequent episodes of loss of awareness. Typically, absence seizures begin in early childhood and remit by teenage years. However, in a minority of patients they persist into adulthood where they are often drug resistant and may be accompanied by other seizure types such as generalised tonic-clonic seizures. In these adult patients, the absence seizures are usually highly disabling, in particular by disqualifying the sufferer from obtaining a motor vehicle license or pursuing occupations and hobbies in which the seizures-associated periods of loss of awareness pose a safety risk and are associated with significant psychosocial disabilities.

While there is a common perception that absence seizures are relatively “easy” to treat, a randomised control trial in patients with childhood absence epilepsy showed that even the most effective anti-epileptic drugs, ethosuximide and valproate, only completely controlled the seizures in 53% and 58% of patients respectively at 16 weeks as assessed by video-EEG recordings, and 45% and 44% respectively at 12 months. Lamotrigine, the other AED commonly used to treat absence seizures, only controlled the seizures in 29% of patients at 16 weeks, and 21% of patients at 12 months. Furthermore, both ethosuximide and valproate are commonly associated with intolerable side effects (occurring in 24% of patients treated with either of these drugs), and the latter is now generally considered to be contraindicated in girls and women of childbearing potential. Other treatment options for absence seizures are limited, with only benzodiazepines having established efficacy- and these are commonly poorly tolerated due to sedative and cognitive side effects. Absence seizures persisting into adult life are particularly difficult to treat, with patients often being treated with multiple drugs resulting in significant side-effects without attaining seizure control.

There is abundant evidence that low threshold (T-type) calcium channels play a critical role in the generation and maintenance of absence seizures, being a key component of the oscillatory burst firing that occurs in thalamocortical neurones during absence seizures. In some embodiments, the present disclosure provides a method of treating absence seizures in a subject in need thereof, the method comprising administering to the subject an effective amount of a salt or a crystalline salt form of Compound 1 or a pharmaceutical composition comprising a salt or a crystalline salt form of Compound 1 described herein. In some embodiments, the absence seizures are refractory absence seizures. In some embodiments, the absence seizures are refractory to an anti-epileptic drug (e.g., ethosuximide, valproic acid, or lamotrigine).

In some embodiments, the subject has epilepsy. In some embodiments, the absence seizures are atypical absence seizures. In some embodiments, the absence seizures comprise adult absence seizures, juvenile absence seizures, or childhood absence seizures.

In some embodiments, the methods described herein further comprise identifying a subject having absence seizures.

In some embodiments, the present disclosure provides a method of treating a generalized epileptic syndrome with absence seizures in a subject in need thereof, the method comprising administering to the subject an effective amount of a salt or a crystalline salt form of Compound 1 or a pharmaceutical composition comprising a salt or a crystalline salt form of Compound 1 described herein. In some embodiments, the method results in reduction of the number of seizures.

In some embodiments, the present disclosure provides a method of treating a generalized epileptic syndrome with absence seizures in a subject in need thereof, the method comprising administering to the subject an effective amount of a salt or a crystalline salt form of Compound 1 or a pharmaceutical composition comprising a salt or a crystalline salt form of Compound 1 described herein.

In some embodiments, the method of treating a generalized epileptic syndrome with absence seizures provided by the present disclosure results in reduction of the mean or total seizure duration.

In some embodiments, the method of treating a generalized epileptic syndrome with absence seizures provided by the present disclosure results in reduction of the seizure frequency, duration or both as measured by Electroencephalogram (EEG).

In some embodiments, the method of treating a generalized epileptic syndrome with absence seizures provided by the present disclosure results in a reduction of the mean seizure duration as measured by EEG.

In some embodiments, the method of treating a generalized epileptic syndrome with absence seizures provided by the present disclosure results in a reduction of the cumulative seizure duration as measured by EEG.

In some embodiments, the method of treating a generalized epileptic syndrome with absence seizures provided by the present disclosure results in a reduction of the total time with 2.5-4 Hz spike wave discharges after hyperventilation and photic stimulation challenges as measured by EEG.

In some embodiments, the method of treating a generalized epileptic syndrome with absence seizures provided by the present disclosure results in a reduction of global severity as measured by Clinical Global Impression-Severity (CGI-S) or Clinical Global Impression-Improvement (CGI-I) scores. CGI-S is a 7-point scale test to rate the severity of the patient's illness at the time of assessment, relative to the clinician's past experience with patients with the same diagnosis. CGI-I is a 7-point scale test to evaluate the improvement of the patient's illness relative to the baseline.

In some embodiments, the method of treating a generalized epileptic syndrome with absence seizures provided by the present disclosure results in a reduction of the number of seizures.

In some embodiments, the method of treating a generalized epileptic syndrome with absence seizures provided by the present disclosure results in a reduction of the seizure density as measured by Electroencephalogram (EEG).

In some embodiments, the method of treating a generalized epileptic syndrome with absence seizures provided by the present disclosure results in a reduction of the mean seizure duration as measured by EEG.

### Genetic Epilepsies

In some embodiments, the present disclosure provides a method of treating epilepsy or an epilepsy syndrome in a subject in need thereof, the method comprising administering to the subject an effective amount of a salt or a crystalline salt form of Compound 1 or a pharmaceutical composition comprising a salt or a crystalline salt form of Compound 1 described herein.

In some embodiments, the epilepsy or epilepsy syndrome is a genetic epilepsy or a genetic epilepsy syndrome. In some embodiments, the epilepsy or epilepsy syndrome is genetic generalized epilepsy. In some embodiments, epilepsy or an epilepsy syndrome comprises epileptic encephalopathy, epileptic encephalopathy with SCN1A, SCN2A, SCN8A mutations, early infantile epileptic encephalopathy, Dravet syndrome, Dravet syndrome with SCN1A mutation, generalized epilepsy with febrile seizures, intractable childhood epilepsy with generalized tonic-clonic seizures, infantile spasms, benign familial neonatal-infantile seizures, SCN2A epileptic encephalopathy, focal epilepsy with SCN3A mutation, cryptogenic pediatric partial epilepsy with SCN3A mutation, SCN8A epileptic encephalopathy, Rasmussen encephalitis, malignant migrating partial seizures of infancy, autosomal dominant nocturnal frontal lobe epilepsy, sudden expected death in epilepsy (SUDEP), KCNQ2 epileptic encephalopathy, and KCNT1 epileptic encephalopathy.

In some embodiments, the methods described herein further comprise identifying a subject having epilepsy or an epilepsy syndrome (e.g., epileptic encephalopathy, epileptic encephalopathy with SCN1A, SCN2A, SCN8A mutations, early infantile epileptic encephalopathy, Dravet syndrome, Dravet syndrome with SCN1A mutation, generalized Epilepsy with febrile seizures, intractable childhood epilepsy with generalized tonic-clonic seizures, infantile spasms, benign familial neonatal-infantile seizures, SCN2A epileptic encephalopathy, focal epilepsy with SCN3A mutation, cryptogenic pediatric partial epilepsy with SCN3A mutation, SCN8A epileptic encephalopathy, Rasmussen encephalitis, malignant migrating partial seizures of infancy, autosomal dominant nocturnal frontal lobe epilepsy, sudden expected death in epilepsy (SUDEP), KCNQ2 epileptic encephalopathy, and KCNT1 epileptic encephalopathy) prior to administration of a salt or a crystalline salt form of Compound 1 or a pharmaceutical composition comprising a salt or a crystalline salt form of Compound 1 described herein.

In one aspect, the present disclosure provides a method of treating epilepsy or an epilepsy syndrome (e.g., epileptic encephalopathy, epileptic encephalopathy with SCN1A, SCN2A, SCN8A mutations, early infantile epileptic encephalopathy, Dravet syndrome, Dravet syndrome with SCN1A mutation, generalized Epilepsy with febrile seizures, intractable childhood epilepsy with generalized tonic-clonic seizures, infantile spasms, benign familial neonatal-infantile seizures, SCN2A epileptic encephalopathy, focal epilepsy with SCN3A mutation, cryptogenic pediatric partial epilepsy with SCN3A mutation, SCN8A epileptic encephalopathy, Rasmussen encephalitis, malignant migrating partial seizures of infancy, autosomal dominant nocturnal frontal lobe epilepsy, sudden expected death in epilepsy (SUDEP), KCNQ2 epileptic encephalopathy, and KCNT1 epileptic encephalopathy) comprising administering to a subject in need thereof an effective amount of a salt or a crystalline salt form of Compound 1 or a pharmaceutical composition comprising a salt or a crystalline salt form of Compound 1 described herein.

A salt or a crystalline salt form of Compound 1 or a pharmaceutical composition comprising a salt or a crystalline salt form of Compound 1 described herein may also be used to treat an epileptic encephalopathy in a subject in need thereof, wherein the subject has a mutation in one or more of ALDH7A1, ALG13, ARHGEF9, ARX, ASAH1, CDKL5, CHD2, CHRNA2, CHRNA4, CHRNB2, CLN8, CNTNAP2, CPA6, CSTB, DEPDC5, DNM1, EEF1A2, EPM2A, EPM2B, GABRA1, GABRB3, GABRG2, GNAO1, GOSR2, GRIN1, GRIN2A, GRIN2B, HCN1, IER3IP1, KCNA2, KCNB1, KCNC1, KCNMA1, KCNQ2, KCNQ3, KCNT1, KCTD7, LGI1, MEF2C, NHLRC1, PCDH19, PLCB1, PNKP, PNPO, PRICKLE1, PRICKLE2, PRRT2, RELN, SCARB2, SCN1A, SCN1B, SCN2A, SCN8A, SCN9A, SIAT9, SIK1, SLC13A5, SLC25A22, SLC2A1, SLC35A2, SLC6A1, SNIP1, SPTAN1, SRPX2, ST3GAL3, STRADA, STX1B, STXBP1, SYN1, SYNGAP1, SZT2, TBC1D24, and WWOX.

In some embodiments, the methods described herein further comprise identifying a subject having a mutation in one or more of ALDH7A1, ALG13, ARHGEF9, ARX, ASAH1, CDKL5, CHD2, CHRNA2, CHRNA4, CHRNB2, CLN8, CNTNAP2, CPA6, CSTB, DEPDC5, DNM1, EEF1A2, EPM2A, EPM2B, GABRA1, GABRB3, GABRG2, GNA01, GOSR2, GRIN1, GRIN2A, GRIN2B, HCN1, IER3IP1, KCNA2, KCNB1, KCNC1, KCNMA1, KCNQ2, KCNQ3, KCNT1, KCTD7, LGI1, MEF2C, NHLRC1, PCDH19, PLCB1, PNKP, PNPO, PRICKLE1, PRICKLE2, PRRT2, RELN, SCARB2, SCN1A, SCN1B, SCN2A, SCN8A, SCN9A, SIAT9, SIK1, SLC13A5, SLC25A22, SLC2A1, SLC35A2, SLC6A1, SNIP1, SPTAN1, SRPX2, ST3GAL3, STRADA, STX1B, STXBP1, SYN1, SYNGAP1, SZT2, TBC1D24, WWOX, CACNA1G, CACNA1H, and CACNA1I prior to administration of a salt or a crystalline salt form of Compound 1 or a pharmaceutical composition comprising a salt or a crystalline salt form of Compound 1 described herein.

A salt or a crystalline salt form of Compound 1 or a pharmaceutical composition comprising a salt or a crystalline salt form of Compound 1 described herein may also be used to treat an epileptic encephalopathy in a subject in need thereof, wherein the subject has a mutation in one or more of ADSL, ALDH5A1, ALDH7A1, ALG13, ARGI, ARHGEF9, ARX, ATP1A2, ATP1A3, ATRX, BRAT1, C12orf57, CACNA1A, CACNA2D2, CARS2, CASK, CDKL5, CHD2, CHRNA2, CHRNA4, CHRNB2, CLCN4, CLN2 (TPP1), CLN3, CLN5, CLN6, CLN8, CNTNAP2, CSTB, CTSD, DDC, DEPDC5, DNAJC5, DNM1, DOCK7, DYRK1A, EEF1A2, EFHC1, EHMT1, EPM2A, FARS2, FOLR1, FOXG1, FRRS1L, GABBR2, GABRA1, GABRB2, GABRB3, GABRG2, GAMT, GATM, GLRA1, GNAO1, GOSR2, GRIN1, GRIN2A, GRIN2B, HCN1, HNRNPU, IER3IP1, IQSEC2, ITPA, JMJD1C, KANSL1, KCNA2, KCNB1, KCNC1, KCNH2, KCNJ10, KCNMA1, KCNQ2, KCNQ3, KCNT1, KCTD7, LGI1, L1AS, MBD5, MECP2, MEF2C, MFSD8, MOCS1, MOCS2, MTOR, NEDD4L, NEXM1F, NGLY1, NHLRC1, NPRL3, NRXN1, PACS1, PCDH19, PIGA, PIGN, PIGO, PLCB1, PNKD, PNKP, PNPO, POLG, PPT1, PRICKLE1, PRIMA1, PRRT2, PURA, QARS, RELN, ROGDI, SATB2, SCARB2, SCN1A, SCN1B, SCN2A, SCN3A, SCN8A, SCN9A, SERPINI1, SGCE, SIK1, SLC12A5, SLC13A5, SLC19A3, SLC25A12, SLC25A22, SLC2A1, SLC35A2, SLC6A1, SLC6A8, SLC9A6, SMC1A, SNX27, SPATA5, SPTAN1, ST3GAL5, STRADA, STX1B, STXBP1, SUOX, SYN1, SYNGAP1, SYNJ1, SZT2, TBC1D24, TCF4, TPK1, TSC1, TSC2, UBE3A, WDR45, WWOX, ZDHHC9, ZEB2, ABAT, ARHGEF15, ATP6AP2, CACNA1H, CACNB4, CASR, CERS1, CNTN2, CPA6, DIAPH1, FASN, GABRD, GAL, GPHN, KCNA1, KCND2, KCNH5, KPNA7, LMNB2, NECAP1, PIGG, PIGQ, PIK3AP1, PRDM8, PRICKLE2, RBFOX1, RBFOX3, RYR3, SCN5A, SETD2, SLC35A3, SNAP25, SRPX2, ST3GAL3, TBLIXRI, AMT, GCSH, GLDC, FLNA, PTEN, and RANBP2.

In some embodiments, the methods described herein further comprise identifying a subject having a mutation in one or more of ADSL, ALDH5A1, ALDH7A1, ALG13, ARGI, ARHGEF9, ARX, ATP1A2, ATP1A3, ATRX, BRAT1, C12orf57, CACNA1A, CACNA2D2, CARS2, CASK, CDKL5, CHD2, CHRNA2, CHRNA4, CHRNB2, CLCN4, CLN2 (TPP1), CLN3, CLN5, CLN6, CLN8, CNTNAP2, CSTB, CTSD, DDC, DEPDC5, DNAJC5, DNM1, DOCK7, DYRK1A, EEF1A2, EFHC1, EHMT1, EPM2A, FARS2, FOLR1, FOXG1, FRRS1L, GABBR2, GABRA1, GABRB2, GABRB3, GABRG2, GAMT, GATM, GLRA1, GNAO1, GOSR2, GRIN1, GRIN2A, GRIN2B, HCN1, HNRNPU, IER3IP1, IQSEC2, ITPA, JMJD1C, KANSL1, KCNA2, KCNB1, KCNC1, KCNH2, KCNJ10, KCNMA1, KCNQ2, KCNQ3, KCNT1, KCTD7, LGI1, LIAS, MBD5, MECP2, MEF2C, MFSD8, MOCS1, MOCS2, MTOR, NEDD4L, NEXMIF, NGLY1, NHLRC1, NPRL3, NRXN1, PACS1, PCDH19, PIGA, PIGN, PIGO, PLCB1, PNKD, PNKP, PNPO, POLG, PPT1, PRICKLE1, PRIMA1, PRRT2, PURA, QARS, RELN, ROGDI, SATB2, SCARB2, SCN1A, SCN1B, SCN2A, SCN3A, SCN8A, SCN9A, SERPINI1, SGCE, SIK1, SLC12A5, SLC13A5, SLC19A3, SLC25A12, SLC25A22, SLC2A1, SLC35A2, SLC6A1, SLC6A8, SLC9A6, SMC1A, SNX27, SPATA5, SPTAN1, ST3GAL5, STRADA, STX1B, STXBP1, SUOX, SYN1, SYNGAP1, SYNJ1, SZT2, TBC1D24, TCF4, TPK1, TSC1, TSC2, UBE3A, WDR45, WWOX, ZDHHC9, ZEB2, ABAT, ARHGEF15, ATP6AP2, CACNA1H, CACNB4, CASR, CERS1, CNTN2, CPA6, DIAPH1, FASN, GABRD, GAL, GPHN, KCNA1, KCND2, KCNH5, KPNA7, LMNB2, NECAP1, PIGG, PIGQ, PIK3AP1, PRDM8, PRICKLE2, RBFOX1, RBFOX3, RYR3, SCN5A, SETD2, SLC35A3, SNAP25, SRPX2, ST3GAL3, TBLIXRI, AMT, GCSH, GLDC, FLNA, PTEN, and RANBP2 prior to administration of a salt or a crystalline salt form of Compound 1 or a pharmaceutical composition comprising a salt or a crystalline salt form of Compound 1 described herein.

A salt or a crystalline salt form of Compound 1 or a pharmaceutical composition comprising a salt or a crystalline salt form of Compound 1 described herein may also be used to treat an epileptic encephalopathy in a subject in need thereof, wherein the subject has a mutation in one or more of ADSL, ALDH5A1, ALDH7A1, ALG13, ARHGEF9, ARX, ASNS, ATP1A2, ATP1A3, ATP6AP2, ATRX, BRAT1, CACNA1A, CASK, CDKL5, CHD2, CHRNA2, CHRNA4, CHRNA7, CHRNB2, CLCN4, CLN3, CLN5, CLN6, CLN8, CNTNAP2, CSTB, CTNNB1, CTSD (CLN10), CTSF, DDX3X, DEPDC5, DNAJC5 (CLN4B), DNM1, DYRK1A, EEF1A2, EHMT1, EPM2A, FLNA, FOLR1, FOXG1, FRRS1L, GABBR2, GABRA1, GABRB2, GABRB3, GABRG2, GAMT, GATM, GLDC, GNAO1, GOSR2, GRIN1, GRIN2A, GRIN2B, HNRNPU, IQSEC2, KANSL1, KCNA2, KCNB1, KCNC1, KCNH1, KCNJ10, KCNMA1, KCNQ2, KCNQ3, KCNT1, KCTD7 (CLN14), KDM6A, KIAA2022, LGI1, MAGI2, MBD5, MECP2, MEF2C, MFSD8 (CLN7), NALCN, NGLY1, NHLRC1 (EPM2B), NPRL3. NR2F1, NRXN1, PACS1, PCDH19, PIGA PIGO, PIGV, PLCB1, PNKP, PNPO, POLG, PPP2R5D, PPT1 (CLN1), PRRT2, PURA, QARS, SATB2, SCARB2, SCN1A, SCN1B, SCN2A, SCN8A, SLC13A5, SLC19A3, SLC25A22, SLC2A1, SLC6A1, SLC6A8, SLC9A6, SMC1A, SPATA5, SPTAN1, STX1B, STXBP1, SYNGAP1, SZT2, TBC1D24, TBLIXRI, TCF4, TPP1 (CLN2), TSC1, TSC2, UBE3A, WDR45, WWOX, and ZEB2.

In some embodiments, the methods described herein further comprise identifying a subject having a mutation in one or more of ADSL, ALDH5A1, ALDH7A1, ALG13, ARHGEF9, ARX, ASNS, ATP1A2, ATP1A3, ATP6AP2, ATRX, BRAT1, CACNA1A, CASK, CDKL5, CHD2, CHRNA2, CHRNA4, CHRNA7, CHRNB2, CLCN4, CLN3, CLN5, CLN6, CLN8, CNTNAP2, CSTB, CTNNB1, CTSD (CLN10), CTSF, DDX3X, DEPDC5, DNAJC5 (CLN4B), DNM1, DYRK1A, EEF1A2, EHMT1, EPM2A, FLNA, FOLR1, FOXG1, FRRS1L, GABBR2, GABRA1, GABRB2, GABRB3, GABRG2, GAMT, GATM, GLDC, GNAO1, GOSR2, GRIN1, GRIN2A, GRIN2B, HNRNPU, IQSEC2, KANSL1, KCNA2, KCNB1, KCNC1, KCNH1, KCNJ10, KCNMA1, KCNQ2, KCNQ3, KCNT1, KCTD7 (CLN14), KDM6A, KIAA2022, LGI1, MAGI2, MBD5, MECP2, MEF2C, MFSD8 (CLN7), NALCN, NGLY1, NHLRC1 (EPM2B), NPRL3. NR2F1, NRXN1, PACS1, PCDH19, PIGA PIGO, PIGV, PLCB1, PNKP, PNPO, POLG, PPP2R5D, PPT1 (CLN1), PRRT2, PURA, QARS, SATB2, SCARB2, SCN1A, SCN1B, SCN2A, SCN8A, SLC13A5, SLC19A3, SLC25A22, SLC2A1, SLC6A1, SLC6A8, SLC9A6, SMC1A, SPATA5, SPTAN1, STX1B, STXBP1, SYNGAP1, SZT2, TBC1D24, TBLIXRI, TCF4, TPP1 (CLN2), TSC1, TSC2, UBE3A, WDR45, WWOX, and ZEB2 prior to administration of a salt or a crystalline salt form of Compound 1 or a pharmaceutical composition comprising a salt or a crystalline salt form of Compound 1 described herein.

A salt or a crystalline salt form of Compound 1 or a pharmaceutical composition comprising a salt or a crystalline salt form of Compound 1 described herein may also be used to treat an epileptic encephalopathy in a subject in need thereof, wherein the subject has a mutation in one or more of ALDH7A1, ARHGEF9, ARX, ATP13A2, ATP1A2, CACNA1A, CASK, CDKL5, CHD2, CHRNA2, CHRNA4, CHRNB2, CLN3, CLN5, CLN6, CLN8, CNTNAP2, CRH, CSTB, CTSD, CTSF, DCX, DEPDC5, DNAJC5, DNM1, DYNC1H1, DYRK1A, EEF1A2, EPM2A, FLNA, FOLR1, FOXG1, GABRA1, GABRB3, GABRG2, GAMT, GATM, GNAO1, GOSR2, GRIN1, GRIN2A, GRIN2B, GRN, HCN1, HNRNPU, IQSEC2, KCNA2, KCNC1, KCNJ10, KCNQ2, KCNQ3, KCNT1, KCTD7, KIAA2022, LGI1, MECP2, MEF2C, MFSD8, NHLRC1, NRXN1, PCDH19, PIGA, PLCB1, PNKP, PNPO, POLG, PPT1, PRICKLE1, PRRT2, PURA, SCARB2, SCN1A, SCN1B, SCN2A, SCN8A, SIK1, SLC13A5, SLC25A22, SLC2A1, SLC35A2, SLC6A1, SLC9A6, SMC1A, SNAP25, SPTAN1, ST3GAL3, STX1B, STXBP1, SYN1, SYNGAP1, SZT2, TBC1D24, TBL1XR1, TCF4, TPP1, TSC1, TSC2, UBE3A, WDR45, and ZEB2.

In some embodiments, the methods described herein further comprise identifying a subject having a mutation in one or more of ALDH7A1, ARHGEF9, ARX, ATP13A2, ATP1A2, CACNA1A, CASK, CDKL5, CHD2, CHRNA2, CHRNA4, CHRNB2, CLN3, CLN5, CLN6, CLN8, CNTNAP2, CRH, CSTB, CTSD, CTSF, DCX, DEPDC5, DNAJC5, DNM1, DYNC1H1, DYRK1A, EEF1A2, EPM2A, FLNA, FOLR1, FOXG1, GABRA1, GABRB3, GABRG2, GAMT, GATM, GNAO1, GOSR2, GRIN1, GRIN2A, GRIN2B, GRN, HCN1, HNRNPU, IQSEC2, KCNA2, KCNC1, KCNJ10, KCNQ2, KCNQ3, KCNT1, KCTD7, KIAA2022, LGI1, MECP2, MEF2C, MFSD8, NHLRC1, NRXN1, PCDH19, PIGA, PLCB1, PNKP, PNPO, POLG, PPT1, PRICKLE1, PRRT2, PURA, SCARB2, SCN1A, SCN1B, SCN2A, SCN8A, SIK1, SLC13A5, SLC25A22, SLC2A1, SLC35A2, SLC6A1, SLC9A6, SMC1A, SNAP25, SPTAN1, ST3GAL3, STX1B, STXBP1, SYN1, SYNGAP1, SZT2, TBC1D24, TBLIXRI, TCF4, TPP1, TSC1, TSC2, UBE3A, WDR45, and ZEB2 prior to administration of a salt or a crystalline salt form of Compound 1 or a pharmaceutical composition comprising a salt or a crystalline salt form of Compound 1 described herein.

### Mood Disorders

In some embodiments, the present disclosure also provides a method of treating a psychiatric disorder that comprise administering to a subject in need thereof a salt or a crystalline salt form of Compound 1 or a pharmaceutical composition comprising a salt or a crystalline salt form of Compound 1 described herein. In some embodiments, the psychiatric disorder may be a mood disorder, for example clinical depression, postnatal depression or postpartum depression, perinatal depression, atypical depression, melancholic depression, psychotic major depression, catatonic depression, seasonal affective disorder, dysthymia, double depression, depressive personality disorder, recurrent brief depression, minor depressive disorder, bipolar disorder or manic depressive disorder, depression caused by chronic medical conditions, treatment-resistant depression, refractory depression, suicidality, suicidal ideation, or suicidal behavior. The methods comprise administering to the subject an effective amount of a salt or a crystalline salt form of Compound 1 or a pharmaceutical composition comprising a salt or a crystalline salt form of Compound 1 described herein. In some embodiments, the methods described herein provide therapeutic effect to a subject suffering from depression (e.g., moderate or severe depression). In some embodiments, the mood disorder is associated with a disease or disorder described herein (e.g., neuroendocrine diseases and disorders, neurodegenerative diseases and disorders (e.g., epilepsy), movement disorders, tremor (e.g., Parkinson's Disease), women's health disorders or conditions).

Clinical depression is also known as major depression, major depressive disorder (MDD), severe depression, unipolar depression, unipolar disorder, and recurrent depression, and refers to a mental disorder characterized by pervasive and persistent low mood that is accompanied by low self-esteem and loss of interest or pleasure in normally enjoyable activities. Some people with clinical depression have trouble sleeping, lose weight, and generally feel agitated and irritable. Clinical depression affects how an individual feels, thinks, and behaves and may lead to a variety of emotional and physical problems. Individuals with clinical depression may have trouble doing day-to-day activities and make an individual feel as if life is not worth living.

Peripartum depression refers to depression in pregnancy. Symptoms include irritability, crying, feeling restless, trouble sleeping, extreme exhaustion (emotional and/or physical), changes in appetite, difficulty focusing, increased anxiety and/or worry, disconnected feeling from baby and/or fetus, and losing interest in formerly pleasurable activities.

Postnatal depression (PND) is also referred to as postpartum depression (PPD) and refers to a type of clinical depression that affects women after childbirth. Symptoms can include sadness, fatigue, changes in sleeping and eating habits, reduced sexual desire, crying episodes, anxiety, and irritability. In some embodiments, the PND is a treatment-resistant depression (e.g., a treatment-resistant depression as described herein). In some embodiments, the PND is refractory depression (e.g., a refractory depression as described herein).

In some embodiments, a subject having PND also experienced depression, or a symptom of depression during pregnancy. This depression is referred to herein as perinatal depression. In an embodiment, a subject experiencing perinatal depression is at increased risk of experiencing PND.

Atypical depression (AD) is characterized by mood reactivity (e.g., paradoxical anhedonia) and positivity, significant weight gain or increased appetite. Patients suffering from AD also may have excessive sleep or somnolence (hypersomnia), a sensation of limb heaviness, and significant social impairment as a consequence of hypersensitivity to perceived interpersonal rejection.

Melancholic depression is characterized by loss of pleasure (anhedonia) in most or all activities, failures to react to pleasurable stimuli, depressed mood more pronounced than that of grief or loss, excessive weight loss, or excessive guilt.

Psychotic major depression (PMD) or psychotic depression refers to a major depressive episode, in particular of melancholic nature, where the individual experiences psychotic symptoms such as delusions and hallucinations.

Catatonic depression refers to major depression involving disturbances of motor behavior and other symptoms. An individual may become mute and stuporose, and either is immobile or exhibits purposeless or bizarre movements.

Seasonal affective disorder (SAD) refers to a type of seasonal depression wherein an individual has seasonal patterns of depressive episodes coming on in the fall or winter.

Dysthymia refers to a condition related to unipolar depression, where the same physical and cognitive problems are evident. They are not as severe and tend to last longer (e.g., at least 2 years).

Double depression refers to fairly depressed mood (dysthymia) that lasts for at least 2 years and is punctuated by periods of major depression.

Depressive Personality Disorder (DPD) refers to a personality disorder with depressive features.

Recurrent Brief Depression (RBD) refers to a condition in which individuals have depressive episodes about once per month, each episode lasting 2 weeks or less and typically less than 2-3 days.

Minor depressive disorder or minor depression refers to a depression in which at least 2 symptoms are present for 2 weeks.

Bipolar disorder or manic depressive disorder causes extreme mood swings that include emotional highs (mania or hypomania) and lows (depression). During periods of mania the individual may feel or act abnormally happy, energetic, or irritable. They often make poorly thought out decisions with little regard to the consequences. The need for sleep is usually reduced. During periods of depression there may be crying, poor eye contact with others, and a negative outlook on life. The risk of suicide among those with the disorder is high at greater than 6% over 20 years, while self-harm occurs in 30-40%. Other mental health issues such as anxiety disorder and substance use disorder are commonly associated with bipolar disorder.

Depression caused by chronic medical conditions refers to depression caused by chronic medical conditions such as cancer or chronic pain, chemotherapy, chronic stress.

Treatment-resistant depression refers to a condition where the individuals have been treated for depression, but the symptoms do not improve. For example, antidepressants or psychological counseling (psychotherapy) do not case depression symptoms for individuals with treatment-resistant depression. In some cases, individuals with treatment-resistant depression improve symptoms, but come back. Refractory depression occurs in patients suffering from depression who are resistant to standard pharmacological treatments, including tricyclic antidepressants, MAOIs, SSRIs, and double and triple uptake inhibitors and/or anxiolytic drugs, as well as non-pharmacological treatments (e.g., psychotherapy, electroconvulsive therapy, vagus nerve stimulation and/or transcranial magnetic stimulation).

Post-surgical depression refers to feelings of depression that follow a surgical procedure (e.g., as a result of having to confront one's mortality). For example, individuals may feel sadness or empty mood persistently, a loss of pleasure or interest in hobbies and activities normally enjoyed, or a persistent felling of worthlessness or hopelessness.

Mood disorder associated with conditions or disorders of women's health refers to mood disorders (e.g., depression) associated with (e.g., resulting from) a condition or disorder of women's health (e.g., as described herein).

Suicidality, suicidal ideation, suicidal behavior refers to the tendency of an individual to commit suicide. Suicidal ideation concerns thoughts about or an unusual preoccupation with suicide. The range of suicidal ideation varies greatly, from e.g., fleeting thoughts to extensive thoughts, detailed planning, role playing, incomplete attempts. Symptoms include talking about suicide, getting the means to commit suicide, withdrawing from social contact, being preoccupied with death, feeling trapped or hopeless about a situation, increasing use of alcohol or drugs, doing risky or self-destructive things, saying goodbye to people as if they won't be seen again.

Symptoms of depression include persistent anxious or sad feelings, feelings of helplessness, hopelessness, pessimism, worthlessness, low energy, restlessness, difficulty sleeping, sleeplessness, irritability, fatigue, motor challenges, loss of interest in pleasurable activities or hobbies, loss of concentration, loss of energy, poor self-esteem, absence of positive thoughts or plans, excessive sleeping, overeating, appetite loss, insomnia, self-harm, thoughts of suicide, and suicide attempts. The presence, severity, frequency, and duration of symptoms may vary on a case-to-case basis. Symptoms of depression, and relief of the same, may be ascertained by a physician or psychologist (e.g., by a mental state examination).

In some embodiments, the mood disorder is selected from depression, major depressive disorder, bipolar disorder, dysthymic disorder, anxiety disorders, stress, post-traumatic stress disorder, bipolar disorder, and compulsive disorders. In some embodiments, the mood disorder is major depressive disorder.

In some embodiments, the method comprises monitoring a subject with a known depression scale, e.g., the Hamilton Depression (HAM-D) scale, the Clinical Global Impression-Improvement Scale (CGI), and the Montgomery-Asberg Depression Rating Scale (MADRS). In some embodiments, a therapeutic effect can be determined by reduction in Hamilton Depression (HAM-D) total score exhibited by the subject. The therapeutic effect can be assessed across a specified treatment period. For example, the therapeutic effect can be determined by a decrease from baseline in HAM-D total score after administering a composition described herein (e.g., 12, 24, or 48 hours after administration; or 24, 48, 72, or 96 hours or more; or 1 day, 2 days, 14 days, 21 days, or 28 days; or 1 week, 2 weeks, 3 weeks, or 4 weeks; or 1 month, 2 months, 6 months, or 10 months; or 1 year, 2 years, or for life).

In some embodiments, the subject has a mild depressive disorder, e.g., mild major depressive disorder. In some embodiments, the subject has a moderate depressive disorder, e.g., moderate major depressive disorder. In some embodiments, the subject has a severe depressive disorder, e.g., severe major depressive disorder. In some embodiments, the subject has a very severe depressive disorder, e.g., very severe major depressive disorder. In some embodiments, the baseline HAM-D total score of the subject (i.e., prior to treatment with a composition described herein), is at least 24. In some embodiments, the baseline HAM-D total score of the subject is at least 18. In some embodiments, the baseline HAM-D total score of the subject is between and including 14 and 18. In some embodiments, the baseline HAM-D total score of the subject is between and including 19 and 22. In some embodiments, the HAM-D total score of the subject before treatment with a composition described herein is greater than or equal to 23. In some embodiments, the baseline score is at least 10, 15, or 20. In some embodiments, the HAM-D total score of the subject after treatment with a composition described herein is about 0 to 10 (e.g., less than 10; 0 to 10, 0 to 6, 0 to 4, 0 to 3, 0 to 2, or 1.8). In some embodiments, the HAM-D total score after treatment with a composition described herein is less than 10, 7, 5, or 3. In some embodiments, the decrease in HAM-D total score is from a baseline score of about 20 to 30 (e.g., 22 to 28, 23 to 27, 24 to 27, 25 to 27, 26 to 27) to a HAM-D total score at about 0 to 10 (e.g., less than 10; 0 to 10, 0 to 6, 0 to 4, 0 to 3, 0 to 2, or 1.8) after treatment with a composition described herein. In some embodiments, the decrease in the baseline HAM-D total score to HAM-D total score after treatment with a composition described herein is at least 1, 2, 3, 4, 5, 7, 10, 25, 40, or 50). In some embodiments, the percentage decrease in the baseline HAM-D total score to HAM-D total score after treatment with a composition described herein is at least 50% (e.g., 60%, 70%, 80%, or 90%). In some embodiments, the therapeutic effect is measured as a decrease in the HAM-D total score after treatment with a composition described herein relative to the baseline HAM-D total score.

In some embodiments, the method of treating a depressive disorder, e.g., major depressive disorder provides a therapeutic effect (e.g., as measured by reduction in Hamilton Depression Score (HAM-D)) within 14, 10, 4, 3, 2, or 1 days, or 24, 20, 16, 12, 10, or 8 hours or less. In some embodiments, the method of treating the depressive disorder, e.g., major depressive disorder, provides a therapeutic effect (e.g., as determined by a statistically significant reduction in HAM-D total score) within the first or second day of the treatment with a composition described herein. In some embodiments, the method of treating the depressive disorder, e.g., major depressive disorder, provides a therapeutic effect (e.g., as determined by a statistically significant reduction in HAM-D total score) within less than or equal to 28 days, e.g., less than or equal to 21 days or less than or equal to 14 days, since the beginning of the treatment with a salt or a crystalline salt form of Compound 1 or a pharmaceutical composition comprising a salt or a crystalline salt form of Compound 1 described herein.

In some embodiments, the therapeutic effect is a decrease from baseline in HAM-D total score after treatment with a salt or a crystalline salt form of Compound 1 or a pharmaceutical composition comprising a salt or a crystalline salt form of Compound 1 described herein. In some embodiments, the HAM-D total score of the subject before treatment with a salt or a crystalline salt form of Compound 1 or a pharmaceutical composition comprising a salt or a crystalline salt form of Compound 1 described herein is at least at least 18, at least 24 or is between 14 and 18. In some embodiments, method for treating a depressive disorder described herein results in a decrease in HAM-D total score of at least 10 or at least 15. In some embodiments, the HAM-D total score of the subject after treatment with a salt or a crystalline salt form of Compound 1 or a pharmaceutical composition comprising a salt or a crystalline salt form of Compound 1 described herein is 8 or less, e.g., 7 or less or 6 or less.

In some embodiments, the method of treating a depressive disorder provided herein provides a therapeutic effect (e.g., as measured by reduction in Clinical Global Impression-Improvement Scale (CGI)). In some embodiments, the reduction in CGI is achieved within 14, 10, 4, 3, 2, or 1 days, or 24, 20, 16, 12, 10, or 8 hours or less of starting treatment with a salt or a crystalline salt form of Compound 1 or a pharmaceutical composition comprising a salt or a crystalline salt form of Compound 1 described herein. In some embodiments, the method of treating the depressive disorder, e.g., major depressive disorder provides a therapeutic effect within the second day of the treatment period. In some embodiments, the therapeutic effect is a decrease from baseline in CGI score at the end of a treatment period (e.g., 14 days after start of administration of a salt or a crystalline salt form of Compound 1 or a pharmaceutical composition comprising a salt or a crystalline salt form of Compound 1 described herein).

In some embodiments, the method of treating a depressive disorder provided herein provides a therapeutic effect (e.g., as measured by a reduction in Montgomery-Asberg Depression Rating Scale (MADRS) score exhibited by the subject. For example, the method provided by the present disclosure results in a reduction of MADRS score within 14, 4, 3, 2, or 1 days; or 96, 84, 72, 60, 48, 24, 20, 16, 12, 10, 8 hours or less after the start of treatment with a salt or a crystalline salt form of Compound 1 or a pharmaceutical composition comprising a salt or a crystalline salt form of Compound 1 described herein. The MADRS is a ten-item diagnostic questionnaire (regarding apparent sadness, reported sadness, inner tension, reduced sleep, reduced appetite, concentration difficulties, lassitude, inability to feel, pessimistic thoughts, and suicidal thoughts) which psychiatrists use to measure the severity of depressive episodes in patients with mood disorders.

### Pain

The present disclosure also provides methods of treating pain that comprise administering to a subject in need thereof a salt or a crystalline salt form of Compound 1 or a pharmaceutical composition comprising a salt or a crystalline salt form of Compound 1 described herein. In some embodiments, the pain comprises acute pain, chronic pain, neuropathic pain, inflammatory pain, nociceptive pain, central pain (e.g., thalamic pain), or migraine. In some embodiments, the pain comprises acute pain or chronic pain. In some embodiments, the pain comprises neuropathic pain, inflammatory pain, or nociceptive pain. In some embodiments, the pain comprises central pain (e.g., thalamic pain). In some embodiments, the pain comprises migraine.

In some embodiments, the methods provided by the present disclosure further comprise identifying a subject having pain, e.g., acute pain, chronic pain, neuropathic pain, inflammatory pain, nociceptive pain, central pain (e.g., thalamic pain), or migraine, prior to administration of a salt or a crystalline salt form of Compound 1 or a pharmaceutical composition comprising a salt or a crystalline salt form of Compound 1 described herein.

### Tremor

The present disclosure also provides method of treating tremor that comprise administering to a subject in need thereof a salt or a crystalline salt form of Compound 1 or a pharmaceutical composition comprising a salt or a crystalline salt form of Compound 1 described herein. In some embodiments, the tremor may be cerebellar tremor or intention tremor, dystonic tremor, essential tremor, orthostatic tremor, parkinsonian tremor, physiological tremor, or rubral tremor. Tremor may include hereditary, degenerative, and idiopathic disorders such as Wilson's disease, Parkinson's disease, and essential tremor, respectively; metabolic diseases; peripheral neuropathies (associated with Charcot-Marie-Tooth, Roussy-Levy, diabetes mellitus, complex regional pain syndrome); toxins (nicotine, mercury, lead, CO, Manganese, arsenic, toluene); drug-induced (neuroleptics tricyclics, lithium, cocaine, alcohol, adrenaline, bronchodilators, theophylline, caffeine, steroids, valproate, amiodarone, thyroid hormones, vincristine); and psychogenic disorders. Clinical tremor can be classified into physiologic tremor, enhanced physiologic tremor, essential tremor syndromes (including classical essential tremor, primary orthostatic tremor, and task- and position-specific tremor), dystonic tremor, parkinsonian tremor, cerebellar tremor, Holmes' tremor (i.e., rubral tremor), palatal tremor, neuropathic tremor, toxic or drug-induced tremor, and psychogenic tremor. In some embodiments, the tremor may be familial tremor.

In some embodiments, the subjects are selected for treatment with a salt or a crystalline salt form of Compound 1 or a pharmaceutical composition of with a salt or crystalline salt form of Compound 1 due to a clinical diagnosis of essential tremor. In some embodiments, the subjects selected for treatment with a salt or a crystalline salt form of Compound 1 or a pharmaceutical composition of with a salt or crystalline salt form of Compound 1 have essential tremor, but do not have intention tremor.

Tremor is an involuntary, rhythmic, muscle contraction and relaxation that can involve oscillations or twitching of one or more body parts (e.g., hands, arms, eyes, face, head, vocal folds, trunk, legs).

Cerebellar tremor or intention tremor is a slow, broad tremor of the extremities that occurs after a purposeful movement. Cerebellar tremor is caused by lesions in or damage to the cerebellum resulting from, e.g., tumor, stroke or other focal lesion disease (e.g., multiple sclerosis)) or a neurodegenerative disease.

Dystonic tremor occurs in individuals affected by dystonia, a movement disorder in which sustained involuntary muscle contractions cause twisting and repetitive motions and/or painful and abnormal postures or positions. Dystonic tremor may affect any muscle in the body. Dystonic tremors occur irregularly and often can be relieved by complete rest or certain sensory maneuvers.

Essential tremor or benign essential tremor is the most common type of tremor. Essential tremor may be mild and non-progressive in some, and may be slowly progressive, starting on one side of the body but typically affecting both sides. The hands are most often affected, but the head, voice, tongue, legs, and trunk may also be involved. Tremor frequency may decrease as the person ages, but severity may increase. Heightened emotion, stress, fever, physical exhaustion, or low blood sugar may trigger tremors and/or increase their severity. Symptoms generally evolve over time and can be both visible and persistent following onset. Tremors, including essential tremor, can interfere with any or all of a person's activities of daily living, such as for example personal hygiene, cooking, eating, dressing, making home repairs, and interacting with other people. Tremors, including essential tremor, can interfere with carcer choice or job performance (e.g., typing on computer or mobile telephone, using tools, sewing, restaurant work (cooking or serving), caring for others (medical or veterinary work), or any work that requires movement may be difficult). Tremors can also have profound emotional effects, such as fear of tremor being discovered, fear of others' reactions, or fear of rejection.

Orthostatic tremor is characterized by fast (e.g., greater than 12 Hz) rhythmic muscle contractions that occurs in the legs and trunk immediately after standing. Cramps are felt in the thighs and legs and the patient may shake uncontrollably when asked to stand in one spot. Orthostatic tremor may occur in patients with essential tremor.

Parkinsonian tremor is caused by damage to structures within the brain that control movement. Parkinsonian tremor is typically seen as a “pill-rolling” action of the hands that may also affect the chin, lips, legs, and trunk. Onset of parkinsonian tremor typically begins after age 60. Movement starts in one limb or on one side of the body and can progress to include the other side.

Rubral tremor is characterized by coarse slow tremor which can be present at rest, at posture, and with intention. The tremor is associated with conditions that affect the red nucleus in the midbrain, such as a stroke.

In some embodiments, the tremor is selected from essential tremor, Parkinson's tremor, or Cerebellar tremor. In some embodiments, the tremor is essential tremor.

In some embodiments, the present disclosure also provides a method of treating essential tremor that comprise administering to a subject in need thereof a salt or a crystalline salt form of Compound 1 or a pharmaceutical composition comprising a salt or a crystalline salt form of Compound 1 described herein. In some embodiments, the method results in reduction of the essential tremor as assessed by The Essential Tremor Rating Assessment Scale (TETRAS) score. The term “The Essential Tremor Rating Assessment Scale (TETRAS)”, as used herein, refers to a scale developed to quantify severity of essential tremor and its impact on daily activities. It has an activities of daily living (ADL) section and a performance section. The ADL section has 12 items rated between 0 to 4, and the performance section has 9 items rated between 0 to 4.

In some embodiments, the reduction of the essential tremor is assessed by The Essential Tremor Rating Assessment Scale (TETRAS) upper limb score.

In some embodiments, the reduction of the essential tremor is assessed by TETRAS performance subscale score or TETRAS performance individual items.

In some embodiments, the subjects treated in accordance with the methods provided by the present disclosure have a moderate essential tremor (i.e., TETRAS score of 10-15). In some embodiments, the subjects treated herein have a TETRAS score of about 10 to about 15, or a TETRAS score of about 12, prior to treatment in accordance with the methods of the present disclosure. Treatment using a salt or a crystalline salt form of Compound 1 or a pharmaceutical composition comprising a salt or a crystalline salt form of Compound 1 described herein may reduce the TETRAS score of the subject. In some embodiments, subjects treated with a salt or a crystalline salt form of Compound 1 or a pharmaceutical composition comprising a salt or a crystalline salt form of Compound 1 described herein experience a mean reduction in TETRAS score of about 2 to 5, e.g., about 3. In some embodiments, subjects treated with a salt or a crystalline salt form of Compound 1 or a pharmaceutical composition comprising a salt or a crystalline salt form of Compound 1 described herein experience a mean reduction in TETRAS score of about 30% to about 50%, e.g., about 40%.

In some embodiments, the present disclosure also provides a method of treating essential tremor that comprise administering to a subject in need thereof a salt or a crystalline salt form of Compound 1 or a pharmaceutical composition comprising a salt or a crystalline salt form of Compound 1 described herein. In some embodiments, the method results in reduction of the essential tremor as assessed by accelerometer-based score, e.g., accelerometer-based upper limb score. In some embodiments, the method results in reduction of the essential tremor as assessed by CGI score.

In some embodiments, the essential tremor is upper limb tremor.

The efficacy of a salt or a crystalline salt form of Compound 1 or a pharmaceutical composition comprising a salt or a crystalline salt form of Compound 1 described herein for treating essential tremor can be measured by the methods described in the following references: Ferreira, J. J. et al., “MDS Evidence-Based Review of Treatments for Essential Tremor.” Mov. Disord. 2019 July; 34 (7): 950-958; Elble, R. et al., “Task Force Report: Scales for Screening and Evaluating Tremor.” Mov. Disord. 2013 November; 28 (13): 1793-800. Deuschl G. et al. “Treatment of patients with essential tremor.” Lancet Neurol. 2011; 10:148-61. Reich S. G. et al. “Essential Tremor.” Med. Clin. N. Am. 103 (2019) 351-356. The disclosures of the references are herein incorporated in their entirety.

### Ataxia

In some embodiments, the present disclosure also provides a method of treating ataxia that comprise administering to a subject in need thereof a salt or a crystalline salt form of Compound 1 or a pharmaceutical composition comprising a salt or a crystalline salt form of Compound 1 described herein. Ataxia, including both cerebellar ataxia and spinal ataxia (e.g., posterior spinal ataxia), generally involves the loss or failure of coordination. Patients exhibiting ataxia may have difficulty regulating the force, range, direction, velocity, and rhythm involved in posture, balance, and limb movement. Ataxia of the trunk, for example, can result in increased postural sway, and an inability to maintain the center of gravity over the base of support. Ataxia and primary or secondary symptoms of ataxic gait and tremor of the limbs may be accompanied by speech disturbance, dysphagia, abnormal ventilation and speech, and involuntary eye movements, dystonia, pyramidal or extrapyramidal symptoms, thereby substantially interfering with the activities of daily life.

As noted above, ataxia may result from a wide range of underlying diseases and conditions in a patient, including cerebellar and neurodegenerative disorders and diseases resulting from chronic or long-term exposure to toxins. Symptoms of ataxia may result from a wide range of diseases, disorders, and environmental factors, including infectious diseases, metabolic diseases, neurodegenerative diseases, genetic diseases, vascular diseases, neoplastic diseases, demyelinating diseases, neuromuscular diseases, and diseases resulting from long-term or chronic exposure to toxins (including drugs and alcohol), among a variety of others; in one embodiment, for example, the ataxia is the result of a metabolic disease, a neurodegenerative disease, a vascular disease, a neuromuscular disease, or a disease resulting from long-term or chronic exposure to toxins. Diseases, disorders, syndromes, and conditions that may result in ataxic symptoms that may be treated according to the methods described herein include, but are not limited to, amyotrophic lateral sclerosis, benign paroxysmal positional vertigo, cerebellar ataxia type 1 (autosomal recessive), cerebellar ataxias (autosomal recessive), cerebellar ataxias (dominant pure), cerebellar cortical atrophy, cerebellar degeneration (subacute), cerebellar dysfunction, cerebellar hypoplasia, cerebellar hypoplasia (endosteal sclerosis), cerebellar hypoplasia (tapetoretinal degeneration), cerebelloparenchymal autosomal recessive disorder 3, cerebelloparenchymal disorder V, cerebellum agenesis (hydrocephaly), cerebral amyloid angiopathy (familial), cerebral palsy, demyelinating disorder, dorsal column conditions, dysautonomia, dysequilibrium syndrome, dysethesis, endocrine diseases, diseases caused by chronic exposure to toxins (e.g., alcohol, drugs, antiepileptics, neuroleptics), Fragile X/Tremor ataxia syndrome, Friedreich's ataxia, frontal lobe dysfunction, genetic diseases, granulomatous angiitis of the central nervous system, Hallervorden-Spatz disease, hereditary motor and sensory neuropathy, hydrocephalus (e.g., low or normal pressure), hypotonia, congenital nystagmus, ataxia and abnormal auditory brainstem response, infantile onset spinocerebellar ataxia, Machado-Joseph disease, Meniere's disease, metabolic disorders, Miller Fisher Syndrome, Minamata disease, multiple sclerosis, muscular dystrophy, Myoclonus-ataxia, neurodegenerative diseases, olivopontocerebellar atrophy, parancoplastic disorders, parkinsonism (atypical), peroncal muscular atrophy, phenyloin toxicity, posterior column ataxia with retinitis pigmentosa, post-polio syndrome, severe damage to the brain (caused by, e.g., head injury, brain surgery, multiple sclerosis or cerebral palsy, chronic alcohol/drug abuse, chronic exposure to toxins, viral infections, or brain tumor), spastic hemiparesis, spastic paraplegia 23, spastic paraplegia glaucoma precocious puberty, SPG, spinocerebellar ataxia, spinocerebellar ataxia (amyotrophy-deafness), spinocerebellar ataxia (dysmorphism), spinocerebellar ataxia 11, spinocerebellar ataxia 17, spinocerebellar ataxia 20, spinocerebellar ataxia 25, spinocerebellar ataxia 29, spinocerebellar ataxia 42, spinocerebellar ataxia 3, spinocerebellar ataxia (autosomal recessive 1), spinocerebellar ataxia (autosomal recessive 3), spinocerebellar ataxia (autosomal recessive 4), spinocerebellar ataxia (autosomal recessive 5), spinocerebellar ataxia (autosomal recessive, with axonal neuropathy), spinocerebellar ataxia (Machado-Joseph type II), spinocerebellar ataxia (X-linked, 2), spinocerebellar ataxia (X-linked, 3), spinocerebellar ataxia (X-linked, 4), spinocerebellar degenerescence (book type), stroke (e.g., acute or hemorrhagic), vertebral artery dissection, vertebral-basilar insufficiency, and diseases caused by vitamin deficiencies, among a variety of others. In one embodiment, the ataxia is the result of a disease selected from Spinocerebellar ataxia, Friedriech's ataxia, and fragile X/tremor ataxia syndrome. In another particular embodiment, the ataxia is the result of Spinocerebellar ataxia or fragile X/tremor ataxia syndrome.

### Tinnitus

In some embodiments, the present disclosure also provides a method of treating tinnitus that comprise administering to a subject in need thereof a salt or a crystalline salt form of Compound 1 or a pharmaceutical composition comprising a salt or a crystalline salt form of Compound 1 described herein. Tinnitus is a condition in which those affected perceive sound in one or both cars or in the head when no external sound is present. Often referred to as “ringing” in the cars, tinnitus can occur intermittently or consistently with a perceived volume ranging from low to painfully high. However, the perceived volume of tinnitus can vary from patient to patient where an objective measure of tinnitus volume in one patient may be perceived as painful but, in another patient, the same volume may be perceived as subtle.

### Sleep Disorders

In some embodiments, the present disclosure also provides a method of treating sleep disorders that comprise administering to a subject in need thereof a salt or a crystalline salt form of Compound 1 or a pharmaceutical composition comprising a salt or a crystalline salt form of Compound 1 described herein. For example, a sleep disorder may be a central disorder of hypersomnolence, narcolepsy type I, narcolepsy type II, idiopathic hypersomnia, Kleine-Levin syndrome, hypersomnia due to a medical disorder, hypersomnia due to a medication or substance, hypersomnia associated with a psychiatric disorder, insufficient sleep syndrome, circadian rhythm sleep-wake disorders, delayed sleep-wake phase disorder, advanced sleep-wake phase disorder, irregular sleep-wake rhythm, non-24-hour sleep-wake rhythm disorder, shift work disorder, jet lag disorder, circadian rhythm sleep-wake disorder not otherwise specified (NOS).

### Combination Therapy

In some embodiments, a salt or a crystalline salt form of Compound 1 or a pharmaceutical composition comprising a salt or a crystalline salt form of Compound 1 described herein may be administered in combination with another agent or therapy. As used herein, the term “in combination” refers to administration to a subject in need thereof of a salt or a crystalline salt form of Compound 1 or a pharmaceutical composition comprising a salt or a crystalline salt form of Compound 1 and of at least one other agent or therapy, or pharmaceutically acceptable salts thereof whereby the subject has an active prescription for the compound of Formula (I) or a pharmaceutically acceptable salt thereof and an active prescription for at least one of one other agent or therapy, or a pharmaceutically acceptable salt thereof, and is being directed by a physician to take the compound of Formula (I) of a pharmaceutically acceptable salt thereof and at least one other active agent or therapy, or a pharmaceutically acceptable salt thereof. In some embodiments, the term “in combination” also refers to administration to a subject in need thereof of a crystalline form of a compound of Formula (I) or a pharmaceutically acceptable salt thereof and of at least one other active agent or therapy, or pharmaceutically acceptable salts thereof over the same period of time. In some embodiments, the crystalline form of a compound of Formula (I) of a pharmaceutically acceptable salt thereof and at least one other active agent or therapy, or a pharmaceutically acceptable salt thereof may be administered in combination to a subject in need thereof each according to the same administration schedule or each according to different administration schedules. For example, in some embodiments, the crystalline form of a compound of Formula (I) of a pharmaceutically acceptable salt thereof and at least one other active agent or therapy, or a pharmaceutically acceptable salt thereof may be each be administered to a subject in need thereof over the same period of time once daily, e.g., in the morning. In other embodiments, the crystalline form of a compound of Formula (I) of a pharmaceutically acceptable salt thereof may be administered to a subject once daily, e.g., in the morning, and at least one other active agent or therapy, or a pharmaceutically acceptable salt thereof may be administered to a subject in need thereof two or three times daily over the same period of time. In some embodiments, the crystalline form of a compound of Formula (I) of a pharmaceutically acceptable salt thereof and at least one other active agent or therapy, or a pharmaceutically acceptable salt thereof may be administered to a subject in need thereof simultaneously as a part of a new pharmaceutical composition. In other embodiments, the crystalline form of a compound of Formula (I) of a pharmaceutically acceptable salt thereof and at least one other active agent or therapy, or a pharmaceutically acceptable salt thereof may be administered in combination simultaneously, or within several minutes or several hours to a subject in need thereof as parts of different pharmaceutical compositions.

In some embodiments, the present disclosure also provides a method of treating ataxia that comprise administering to a subject in need thereof a salt or a crystalline salt form of Compound 1 or a pharmaceutical composition comprising a salt or a crystalline salt form of Compound 1 described herein may have a disease, disorder, or condition, or a symptom thereof, that would benefit from treatment with another agent or therapy. These diseases or conditions can relate to epilepsy or an epilepsy syndrome (e.g., absence seizures, juvenile myoclonic epilepsy, or a genetic epilepsy) or tremor (e.g., essential tremor).

### Antiepilepsy Agents

Anti-epilepsy agents include brivaracetam, carbamazepine, clobazam, clonazepam, diazepam, divalproex, eslicarbazepine, ethosuximide, ezogabine, felbamate, gabapentin, lacosamide, lamotrigine, levetiracetam, lorazepam, oxcarbezepine, permpanel, phenobarbital, phenytoin, pregabalin, primidone, rufinamide, tigabine, topiramate, valproic acid, vigabatrin, zonisamide.

### Analgesics

Analgesics are therapeutic agents that are used to relieve pain. Examples of analgesics include opiates and morphinomimetics, such as fentanyl and morphine; paracetamol; NSAIDs, and COX-2 inhibitors. Given the ability of Compound 1 to treat pain via inhibition of T-type calcium channels (e.g., Cav3.1, Cav3.2, and Cav3.3), combination with analgesics is particularly envisioned.

### Tremor Medications

Tremor medications include propranolol, primidone, clonazepam, diazepam, lorazepam, alprazolam, gabapentin, topiramate, topamax, neurontin, atenolol, klonopin, alprazolam, nebivolol, carbidopa/levodopa, clonazepam, hydrochlorothiazide/metoprolol, gabapentin enacarbil, labetalol, lactulose, lamotrigine, metoprolol, nadolol, hydrochlorothiazide, and zonisamide.

### EXAMPLES

### Example 1. Salt Screen of Compound 1

The goal of this experiment was to generate various salts of Compound 1 and to determine if any of the resulting salts were crystalline salts. For the experiment, Compound 1 was mixed with acids under various conditions as shown in Table 19, and the resulting products were characterized by XRPD (the XRPD spectra are not shown). Several samples were identified which were characterized by unique XRPD patterns, i.e., the XRPD patterns that did not contain peaks from the starting materials. In cases of fumarate, glutarate, maleate, orotate and oxalate salts of Compound 1, two different XRPD patterns were obtained for one salt, indicating that these salts are polymorphic.

TABLE 19
Salts of Compound 1

| Acid | Salt Name | Conditions^{a} | XRPD Pattern^{b} |
|---|---|---|---|
| Acetic | Acetate | E, EtOH | API A (PO) |
| | | Grind, ACN | API A |
| | | LY, dioxane | NC |
| Adipic | adipate | E, EtOH | New + API A + acid |
| | | SL, ACN:water (9:1), RT; IS | — |
| | | Grind, ACN | API A + acid |
| Alginic | Alginate | SL, ACN/water (9:1), RT; IS | — |
| | | Grind, IPA | API A |
| | | LY, dioxane/water | NC |
| L-ascorbic | Ascorbate | E, EtOH | API A (PO) |
| | | Grind, ACN | API A + acid |
| | | LY, dioxane/water | NC |
| L-aspartic | Aspratate | SL, acetone, 50° C. | Acid |
| | | Grind, ACN | API A + acid |
| | | LY, dioxane/water | NC + pks |
| Benzenesulfonic | Besylate | E, acetone, RT | — |
| | | C, EtOH, 50° C. → -15° C.; NS | New + API A |
| | | SL, IPE,. 50° C. | API A + NC |
| Benzoic | Benzoate | E, EtOH | API A + NC |
| | | Grind, ACN | API A + NC |
| | | SL, IPE, RT | API A |
| (+)-camphoric | Camphorate | E, EtOH | API A (PO) |
| | | Grind, ACN | API A + acid |
| | | E, MeOH/heptane (1:1), RT; gel | — |
| Capric | Caprate | E, EtOH | API A (PO) |
| | | Grind, ACN | API A + pks |
| | | LY, dioxane; oil | — |
| Caprylic | Caprylate | E, EtOH | API A (PO) |
| | | Grind, ACN | API A |
| | | LY, dioxane; oil | — |
| Citric | Citrate | E, MeOH/toluene (1:1); oil | — |
| | | Grind, EtOAc | API A + acid + NC |
| Cyclamic | Cyclamate | P, DCM | New + API A |
| | | E, EtOH/toluene (1:1); oil | — |
| Ethane-1,2- | Edisylate | P, DCM | New |
| disulfonic | | E, EtOH/isopropyl acetate (1:2); oil | — |
| Ethanesulfonic | Esylate | E, acetone/IPE (1:2) | New |
| | | E, MeOH/ACN (1:2); oil | — |
| 2-hydroxy- | Isethionate | E, acetone/toluene (1:1), RT; oil | — |
| ethanesulfonic | | Grind, ACN | API A + NC |
| Formic | Formate | E, EtOH | API A (PO) |
| | | Grind, ACN | API A |
| | | E, MeOH/IPE (1:2); oil | — |
| Fumaric | Fumarate | E, MeOH/toluene (1:1), RT | New 1 + API A |
| | | Grind, EtOAc | New 2 + Acid |
| Galactaric | Mucate | SL, ACN, 50° C. | API A + acid |
| (mucic) | | Grind, IPA | API A + acid |
| | | LY, dioxane/water; oil | — |
| Gentisic | Gentisate | E, acetone/toluene (1:1), RT; oil | — |
| | | Grind, BuOAc | API A + NC |
| D-glucoheptonic | Glucoheptanate | E, MeOH/THF (1:1), RT; oil | — |
| | | E, MeOH/DCM (1:1), RT; oil | — |
| D-gluconic | Gluconate | E, MeOH/toluene (1:1), RT | API A |
| | | Grind, ACN | API A |
| | | LY, dioxane/water | API A + NC |
| D-glucuronic | Glucuronate | P, DCM | NC |
| | | Grind, acetone | API A + pks |
| | | LY, dioxane/water | API A + NC |
| L-glutamic | Glutamate | SL, ACN, 50° C. | API A + acid |
| | | Grind, ACN/water (9:1) | API A + acid |
| | | LY, dioxane/water | NC |
| Glutaric | Glutarate | E, EtOH | New 1 + NC |
| | | Grind, ACN | New 2 |
| | | E, EtOH/butyl acetate (1:2); oil | — |
| Ketoglutaric | Ketoglutarate | E, MeOH/IPE (1:1) | NC |
| | | Grind, ACN | API A + NC |
| | | E, MeOH/ACN (1:1) | NC + pks |
| Glycolic | Glycolate | P, DCM | New + API A |
| | | E, EtOH/ACN | API A pks |
| | | Grind, EtOH | New + API A |
| Hippuric | Hippurate | E, EtOH | API A + NC |
| | | Grind, IPA | API A + acid |
| | | LY, dioxane/water; oil | — |
| Isobutyric | Isobutyrate | E, MeOH | API A |
| | | Grind, ACN | API A |
| | | LY, dioxane; oil | — |
| D,L-lactic | Lactate | C, acetone, RT → −15° C.; NS | — |
| | | LY, dioxane; oil | — |
| | | E, MeOH/ACN (1:1); oil | — |
| Lactobionic | Lactobionate | P, THE, RT; oil | — |
| | | Grind, IPA | API A + NC |
| | | LY, dioxane/water | API A + NC |
| Lauric | Laurate | E, MeOH | API A + acid + pks |
| (dodecanoic) | | Grind, ACN | API A + acid + pks |
| | | LY, dioxane; oil | — |
| Maleic | Maleate | E, MeOH/ACN (1:5) | API A + acid |
| | | Grind, EtOAc | New 1 + API A |
| | | Grind, EtOAc | New 2 + API A + acid |
| | | E, EtOAc; oil | — |
| L-malic | Malate | E, EtOAc; oil | — |
| | | Grind, IPA | API A + NC |
| Malonic | Malonate | C, acetone, RT → −15° C., NS | — |
| | | Grind, BuOAc | New |
| Methanesulfonic | Mesylate | C, acetone, RT → −15° C., NS | — |
| | | E, MeOH:IPE (1:1) | New (disordered) |
| | | E, MeOH/ACN (1:2); oil | — |
| Naphthalene-1,5- | Napadisylate | P, THF | New (disordered) |
| disulfonic | | P, MeOH/DCE (1:5) | New |
| Naphthalene-2- | Napsylate | SL, MeOH:IPE (1:3), RT | New |
| sulfonic | | C, DCE, RT → −15° C., NS | — |
| 1-hydroxy-2- | Xinafoate | C, acetone, RT → −15° C., NS | — |
| napthoic | | C, DCE, RT RT → −15° C., NS | — |
| | | E, EtOH/toluene (1:1); oil | |
| Nicotinic | Nicotinate | E, MeOH | API A + acid |
| | | Grind, ACN | API A + acid |
| | | SL, ACN, 50° C. | Acid |
| Oleic | Oleate | E, MeOH | API A (PO) |
| | | Grind, ACN | API A + NC |
| | | LY, dioxane; oil | — |
| Orotic | Orotate | SL, MeOH | Acid |
| | | Grind, ACN | New 1 + API A |
| | | Grind, IPA | New 2 |
| Oxalic | Oxalate | P, THF | New 1 (disordered) |
| | | E, MeOH/ACN (1:1) | New 2 + API A |
| | | | (min) |
| Palmitic | Palmitate | E, MeOH | API A + acid |
| | | Grind, ACN | API A + acid |
| | | LY, dioxane; oil | — |
| Pamoic | Pamoate | SL, chloroform; NS. FE | API A + acid + pks |
| | | SL, ACN, RT | New |
| Phosphoric | Phosphate | P, MeOH:IPE (1:2) | New + NC |
| | | C, DCM → −15° C.; NS | — |
| | | E, MeOH:IPE (1:1); oil | — |
| Propionic | Propionate | E, MeOH | API A |
| | | Grind, ACN | API A |
| | | LY, dioxane; oil | — |
| L-pyroglutamic | Pyroglutamate | C, acetone, RT → −15° C.; NS | — |
| | | Grind, MEK | API A + pks |
| Sebacic | Sebacate | E, MeOH | API A + acid |
| | | Grind, ACN | API A + acid |
| | | LY, dioxane | New pks + acid + NC |
| Stearic | Stearate | E, MeOH | API A + acid |
| | | Grind, ACN | API A + acid |
| | | LY, dioxane; oil | — |
| Succinic | Succinate | E, MeOH; oil; E, ACN | API A + NC |
| | | Grind, ACN | API A + acid |
| | | C, EtOAc, RT → −15° C.; NS | — |
| Sulfuric | Sulfate | C, EtOAc, RT → −15° C.; NS | — |
| | | P, DCM, SE: oil | — |
| L-tartaric | Tartrate | C, acetone, RT → −15° C.; NS | — |
| | | Grind, IPA | API A + acid + NC |
| | | 50° C. slurry, ACN; gel | |

^{a}C = cool; E = evaporation; EtOH—absolute etanol; IS = insufficient solid; LY = lyophilization; P = precipitation; RT = room temperature; SL = slurry; ACN = acetonitrile; DCM = dichloromethane, MEK = methyl ethyl ketone, IPE = isopropyl ether, IPA = isopropyl alcohol.
^{b}API A = Compound 1 form A (amorphous); NC = non-crystalline]; pks = unidentified peaks; PO = preferred orientation, New = new crystalline salt, New 1 and New 2 = two different crystalline forms of a new salt.

Selected salts having a unique XRPD pattern were additionally analyzed by proton NMR spectroscopy (spectra are not shown). The results are summarized in Table 20 below. Materials that appeared to contain unreacted starting materials were not analyzed.

TABLE 20
Characterization Data for Crystalline Salts of Compound 1

| Salt | Results |
|---|---|
| Edisylate | Consistent with 2:1 salt (API:acid) |
| Esylate | Consistent with 1:1 salt |
| Glutarate | Consistent with 1:1 salt |
| Napadisylate | Consistent with 2:1 salt (API:acid) |
| Napsylate | Consistent with 1:1 salt, impurities noted, trace IPE present |
| Orotate | Consistent with 1:1 salt, residual IPA present |
| Maleate | Crystalline, contains API and acid |
| Malonate | Consistent with 1:1 salt, trace BuOAc present |
| Mesylate | Crystalline, stiochiometry not determined |
| Pamoate | Consistent with 1:1 salt |
| Adipate | Crystalline, contains API and acid |
| Besylate | Crystalline, contains API |
| Cyclamate | Crystalline, contains API |
| Fumarate | Consistent with 1:1 salt |
| Glycolate | Crystalline, contains API |
| Oxalate | Consisent with salt formation, impurities noted |
| Phosphate | Consisent with salt formation, impurities noted |

### Methods

The typical procedures used to prepare and characterize the salts listed in Table 19 are described below.

### Typical Cooling Procedure

This procedure was used, e.g., in an experiment aimed at generating besylate salt of Compound 1. To a 1-dram glass vial were added 24.6 mg (0.064 mmol) of Compound 1 freebase, 10.2 mg of benzenesulfonic acid (0.064 mmol), and 1 mL of ethyl acetate. The mixture was heated to approximately 60° C. on a hot plate. Once all of the solid dissolved, the heat was turned off and the solution was allowed to cool to room temperature. The sample was left at room temperature for 3 days, during which time no crystallization occurred. The sample was then placed in a refrigerator (˜2°) for 2 days, during which time no crystallization occurred. The sample was then placed in a freezer (˜−15° C.) for 7 days, during which time no crystallization occurred. None of the cooling experiments in the screen produced solids.

### Typical Evaporation Procedure

This procedure was used, e.g., in an experiment aimed at generating esylate salt of Compound 1. To a 1-dram glass vial were added 25.0 mg (0.065 mmol) of Compound 1 freebase, 7.4 mg of ethanesulfonic acid (0.067 mmol), and 0.5 mL of acetone. The mixture was sonicated to give a solution. Aluminum foil was placed over the opening of the vial and one pinhole was added. The vial was left in a fume hood at ambient temperature and the solvent was allowed to evaporate, resulting in solid, which was subsequently analyzed by XRPD.

### Typical Grinding Procedure

This procedure was used, e.g., in an experiment aimed at generating glutarate salt of Compound 1. To a PEEK grinding cup were added 25.2 mg (0.066 mmol) of Compound 1 freebase, 8.7 mg of glutaric acid (0.066 mmol), 10 μL of acetonitrile, and a steel ball. The inside volume of the grinding cup is approximately 1.5 mL. The cup was placed on a Retsch mill and the sample was milled at 100% power for 30 minutes. The resulting solid was analyzed by XRPD.

### Typical Lyophilization Procedure

This procedure was used, e.g., in an experiment aimed at generating sebacate salt of Compound 1 freebase. To a 50-mL round-bottom flask were added 24.9 mg (0.065 mmol) of Compound 1, 12.6 mg of sebacic acid (0.063 mmol), and 3 mL of 1,4-dioxane to give a solution. The flask was rotated in a dry-ice acetone bath to freeze the solution to the sides of the flask.

The flask was placed on a Labconco FreeZone 1 lyophilizer overnight to encourage crystallization of the presumably-amorphous lyophile, resulting in solid. The flask was placed in a 60° C. oven overnight and subsequently analyzed by XRPD.

### Typical Slurry Procedure

This procedure was used, e.g., in an experiment aimed at generating pamoate salt of Compound 1 freebase. To a 1-dram glass vial were added 25.1 mg (0.065 mmol) of Compound 1, 25.9 mg of pamoic acid (0.067 mmol), and 1 mL of acetonitrile. The resulting slurry was stirred at room temperature for 3 days. The vial was centrifuged, the mother liquor was decanted, and the solid was allowed to air dry in a fume hood. After drying, the solid was analyzed by XRPD.

### Typical Precipitation Procedure

This procedure was used, e.g., in an experiment aimed at generating edisylate salt of Compound 1 freebase. A solution of 25.0 mg (0.065 mmol) of Compound 1 in 1 mL of dichloromethane and a solution of 12.0 mg of ethane-1,2-disulfonic acid (0.063 mmol in 1 mL of dichloromethane) were combined. Solid precipitated and the resulting slurry was stirred magnetically at room temperature for 1 day. The vial was centrifuged, the mother liquor was decanted, and the solid was allowed to air dry in a fume hood. After drying, the solid was analyzed by XRPD.

### X-Ray Powder Diffraction (XRPD)

XRPD was used to characterize salts of Compound 1 that were produced as described in Table 19. The Rigaku Smart-Lab X-ray diffraction system was configured for reflection Bragg-Brentano geometry using a line source X-ray beam. The x-ray source is a Cu Long Fine Focus tube that was operated at 40 kV and 44 ma. That source provides an incident beam profile at the sample that changes from a narrow line at high angles to a broad rectangle at low angles. Beam conditioning slits are used on the line X-ray source to ensure that the maximum beam size is less than 10 mm both along the line and normal to the line. The Bragg-Brentano geometry is a para-focusing geometry controlled by passive divergence and receiving slits with the sample itself acting as the focusing component for the optics. The inherent resolution of Bragg-Brentano geometry is governed in part by the diffractometer radius and the width of the receiving slit used. Typically, the Rigaku Smart-Lab is operated to give peak widths of 0.1° 2θ or less. The axial divergence of the X-ray beam is controlled by 5.0-degree Soller slits in both the incident and diffracted beam paths.

Powder samples were prepared in a low background Si holder using light manual pressure to keep the sample surfaces flat and level with the reference surface of the sample holder. Each sample was analyzed from 2 to 40° 2θ using a continuous scan of 6° 2θ per minute with an effective step size of 0.02° 2θ.

### Nuclear Magnetic Resonance (NMR) Spectroscopy

The ¹H NMR spectra were acquired on a Bruker Avance II 400 spectrometer. Samples were prepared by dissolving material in DMSO-d₆. The solutions were placed into individual 5-mm NMR tubes for subsequent spectral acquisition. The temperature controlled (295K) 1H NMR spectra acquired on the Avance II 400 utilized a 5-mm cryoprobe operating at an observing frequency of 400.18 MHz.

Tables 19 and 20 demonstrate successful synthesis of the following salts of Compound 1: acetate, adipate, alginate, ascorbate, asparatate, besylate, benzoate, citrate, cyclamate, edisylate, esylate, isethionate, fumarate, gentisate, gluconate, glucuronate, glutamate, glutarate, ketoglutarate, glycolate, hippurate, lactobionate, maleate, malate, malonate, mesylate, napadisylate, napsylate, oleate, oroate, oxalate, pamoate, phosphate, sebacate, succinate and tartrate.

Of the above salts, acetate, alginate, ascorbate, aspartate, besylate, benzoate, citrate, isethionate, gentisate, gluconate, glucuronate, glutamate, ketoglutarate, hippurate, lactobionate, malate, oleate, phosphate, sebacate, succinate and tartrate salts were synthesized as non-crystalline forms, as evidenced by their XRPD spectra. These salts are indicated in Table 19 as “NC”

Of the above salts, adipate, besylate, cyclamate, edisylate, esylate, fumarate, glutarate, glycolate, maleate, malonate, mesylate, napadisylate, napsylate, oroate, oxalate, pamoate and phosphate salts were synthesized as crystalline forms, as evidenced by their XRPD spectra. These salts are indicated in Table 19 as “New”, “New 1” or “New 2”.

### Example 2. Preparation and Characterization of Edisylate, Maleate, Malonate, Mesylate and Pamoate Crystalline Salts of Compound 1

This experiment was a follow-up to the salt screen. The goal of the experiment was to prepare again and characterize the following crystalline salts of Compound 1: edisylate, maleate, malonate, mesylate, and pamoate.

### Preparation of Edisylate Crystalline Salt of Compound 1

Three different procedures used for synthesizing edisylate crystalline salt of Compound 1 are described below.

### Procedure 1

Compound 1 in the amount of 103.1 mg was dissolved in 4 mL of dichloromethane and 1 equivalent of ethane-1,2-disulfonic acid dihydrate (60.4 mg) was dissolved in 0.7 mL of methanol. The solutions were combined in a glass vial, resulting in a clear solution. The solution was stirred at room temperature for 1 day and remained clear. The sample was transferred to a freezer (−20° C.), and stirring was continued. Solid was observed after 1 day. Hexanes in the amount of 1 mL was added and the stirring was continued in the freezer for an additional 3 days. The sample was centrifuged, the mother liquor decanted, and the solid allowed to air-dry prior to XRPD analysis.

### Procedure 2

Compound 1 in the amount of 102.8 mg was dissolved in 1 mL of dichloromethane and 1 equivalent of ethane-1,2-disulfonic acid dihydrate (64.1 mg) was dissolved in 0.2 mL of methanol. The solutions were combined in a glass vial, resulting in a clear solution. The solution was stirred at room temperature and solids were noted within 10 minutes. Stirring at room temperature was continued for 4 days. The sample was centrifuged, the mother liquor decanted, and the solid was allowed to air-dry prior to XRPD analysis.

### Procedure 3

The solids resulting from Procedure 2 were combined with 1 mL of acetonitrile. The resulting slurry was stirred at room temperature for 3 days. The sample was centrifuged, the mother liquor decanted, and the solid allowed to air-dry prior to XRPD analysis.

FIG. 19 shows the results of DSC and TGA analysis of the edisylate crystalline salt of Compound 1. The results of the analysis indicate that Form 1 dehydrates at around 75° C. followed by melting at around 176° C. The weight loss through the melt may be due to the salt breaking and decomposition of ethane-1,2-disulfonic acid.

FIG. 20 shows the results of the NMR analysis of the edisylate crystalline salt of Compound 1. The results of the analysis indicate that Form 1 is a possible di-hydrate with a stoichiometry of 2:1 API:acid.

### Preparation of Maleate Crystalline Salt of Compound 1

Compound 1 in the amount of 101.3 mg was combined with 1 equivalent of maleic acid (30.4 mg) and 4 mL of acetonitrile in a glass vial. The resulting slurry was stirred at room temperature, becoming clear within 2 minutes. Solid precipitated within 4 hours. The slurry was stirred at room temperature for 5 days. The sample was centrifuged, the mother liquor decanted, and the solid allowed to air-dry prior to XRPD analysis.

FIG. 21 shows the results of DSC and TGA analysis of the maleate crystalline salt of Compound 1. The results of the analysis indicate that Form 7 melts at around 161° C.

FIG. 22 shows the results of the NMR analysis of the maleate crystalline salt of Compound 1. The results of the analysis indicate that Form 7 is unsolvated with a stoichiometry of 1:1 API:acid.

### Preparation of Malonate Crystalline Salt of Compound 1

Compound 1 in the amount of 100.8 mg was combined with 1 equivalent of malonic acid (27.5 mg) and 4 mL of acetonitrile in a glass vial. The resulting slurry was stirred at room temperature, becoming clear within 2 minutes. Solid precipitated within 5 minutes. The slurry was stirred at room temperature for 5 days. The sample was centrifuged, the mother liquor decanted, and the solid allowed to air-dry prior to XRPD analysis.

FIG. 23 shows the results of DSC and TGA analysis of the malonate crystalline salt of Compound 1. The results of the analysis indicate that Form 8 melts at around 161° C. The weight loss through the melt may be due to the salt breaking and decomposition of malonic acid.

FIG. 24 shows the results of the NMR analysis of the malonate crystalline salt of Compound 1. The results of the analysis indicate that Form 8 is unsolvated with a stoichiometry of 1:1 API:acid.

### Preparation of Mesylate Crystalline Salt of Compound 1

Three different procedures used for synthesizing edisylate crystalline salt of Compound 1 are described below.

### Procedure 1

Compound 1 in the amount of 101.9 mg was added to a solution of 1 mL of methanol containing 1 equivalent of methanesulfonic acid (25.0 mg) in a glass vial. The slurry was stirred at room temperature and the solid dissolved. A 1 mL aliquot of isopropyl ether was added twice per day. After addition of 7 mL of isopropyl ether (after about 3 days), the solution remained clear. The sample was placed in a refrigerator (5° C.) for 3 days and remained clear. The sample was removed from the refrigerator and left uncapped in a fume hood for the solvent to evaporate. To the resulting oil was added 10 mL of diethyl ether and the sample was sonicated which resulted in precipitation of a white solid. The slurry was stirred at room temperature for 1 day. The sample was centrifuged, the mother liquor decanted, and the solid allowed to air-dry prior to XRPD analysis.

### Procedure 2

Compound 1 in the amount of 101.4 mg was added to a solution of 0.5 mL of methanol containing 1 equivalent of methanesulfonic acid (25.8 mg) in a glass vial. The slurry was stirred at room temperature and the solid dissolved. Added to the solution was 1 mL of isopropyl ether, and the solution remained clear after stirring overnight. 1 mL of isopropyl ether was added every 2 hours. After three aliquots (3 mL), a clear oil formed. Stirring was continued for 3 days and the oil remained. The solvent was evaporated using a dry air purge, resulting in a white solid. The sample was heated at 60° C. overnight prior to XRPD analysis.

### Procedure 3

To the solids resulting from Procedure 2, 1 mL of acetonitrile was added. The resulting slurry was stirred at room temperature and became clear within minutes. The sample was cooled to −15° C. and remained clear. The sample was warmed to room temperature and 3 mL of tert-butyl methyl ether was added. A precipitate formed within 90 minutes. The slurry was stirred for 2 days. The sample was centrifuged, the mother liquor decanted, and the solid was allowed to air-dry prior to XRPD analysis.

FIG. 25 shows the results of DSC and TGA analysis of the mesylate crystalline salt of Compound 1. The results of the analysis indicate that Form 9 melts at around 190° C.

FIG. 26 shows the results of the NMR analysis of the mesylate crystalline salt of Compound 1. The results of the analysis indicate that Form 9 is unsolvated with a stoichiometry of 1:1 API:acid.

### Synthesis of Pamoate Crystalline Salt of Compound 1

Compound 1 in the amount of 102.2 mg was combined with 1 equivalent of pamoic acid (59.9 mg), and 4 mL of acetonitrile in a glass vial. The resulting slurry was stirred at room temperature for 6 days. The sample was centrifuged, the mother liquor decanted, and the solid allowed to air-dry prior to XRPD analysis.

FIG. 27 shows the results of DSC and TGA analysis of the pamoate crystalline salt of Compound 1. The results of the analysis indicate that Form 10 melts at around 225° C.

FIG. 28 shows the results of the NMR analysis of the pamoate crystalline salt of Compound 1. The results of the analysis indicate that Form 10 is unsolvated with a stoichiometry of 1:1 API:acid.

### Characterization of Crystalline Salts of Compound 1

The crystalline salts of Compound 1 were characterized by X-ray powder diffraction (XRPD), differential scanning calorimetry (DSC), thermogravimetric analysis (TGA) and nuclear magnetic resonance (NMR) spectroscopy. The general procedures used for each analysis are described below.

### X-Ray Powder Diffraction (XRPD)

The Rigaku Smart-Lab X-ray diffraction system was configured for reflection Bragg-Brentano geometry using a line source X-ray beam. The x-ray source is a Cu Long Fine Focus tube that was operated at 40 kV and 44 ma. That source provides an incident beam profile at the sample that changes from a narrow line at high angles to a broad rectangle at low angles. Beam conditioning slits are used on the line X-ray source to ensure that the maximum beam size is less than 10 mm both along the line and normal to the line.

The Bragg-Brentano geometry is a para-focusing geometry controlled by passive divergence and receiving slits with the sample itself acting as the focusing component for the optics. The inherent resolution of Bragg-Brentano geometry is governed in part by the diffractometer radius and the width of the receiving slit used. Typically, the Rigaku Smart-Lab is operated to give peak widths of 0.1° 2θ or less. The axial divergence of the X-ray beam is controlled by 5.0-degree Soller slits in both the incident and diffracted beam paths.

Powder samples were prepared in a low background Si holder using light manual pressure to keep the sample surfaces flat and level with the reference surface of the sample holder. Each sample was analyzed from 2 to 40° 2θ using a continuous scan of 6° 2θ per minute with an effective step size of 0.02° 2θ.

### Differential Scanning Calorimetry (DSC)

DSC analyses were carried out using a TA Instruments Q2500 Discovery Series instrument. The instrument temperature calibration was performed using indium. The DSC cell was kept under a nitrogen purge of ˜50 mL per minute during each analysis. The sample was placed in a standard, crimped, aluminum pan and was heated from approximately 25° C. to 350° C. at a rate of 10° C. per minute.

### Thermogravimetric Analysis (TGA)

The TG analysis was carried out using a TA Instruments Q5500 Discovery Series instrument. The instrument balance was calibrated using class M weights and the temperature calibration was performed using alumel. The nitrogen purge was ˜40 mL per minute at the balance and ˜60 mL per minute at the furnace. Each sample was placed into a pre-tared platinum pan and heated from approximately 25° C. to 350° C. at a rate of 10° C. per minute.

### Nuclear Magnetic Resonance (NMR) Spectroscopy

The ¹H NMR spectra were acquired on a Bruker Avance II 400 spectrometer. Samples were prepared by dissolving material in DMSO-d₆. The solutions were placed into individual 5-mm NMR tubes for subsequent spectral acquisition. The temperature controlled (295K) 1H NMR spectra acquired on the Avance II 400 utilized a 5-mm cryoprobe operating at an observing frequency of 400.18 MHz.

### Results

Table 21 below contains a summary of the preparation and characterization of the crystalline salts of Compound 1.

TABLE 21
Characteristics of Crystalline Salts of Compound 1

| Salt | Properties |
|---|---|
| Edisylate | One crystalline phase, designated as Form 1, was identified |
| | in the salt screen. The stoichiometry was determined to be |
| | 2:1 API:acid by NMR. |
| | Form 1: prepared using procedure 3; crystalline, possible di- |
| | hydrate, stoichiometry is 2:1 API:acid (NMR). DSC data |
| | suggests that it dehydrates around 75° C. followed by melting |
| | around 176° C. |
| Maleate | One crystalline phase, designated as Form 7, was identified |
| | in the salt screen. Form 7: crystalline, unsolvated, |
| | stoichiometry is 1:1 (NMR), melts around 161° C. (DSC). |
| Malonate | One crystalline phase, designated as form 8, was identified in |
| | the salt screen. Form 8: crystalline, unsolvated, soichiometry |
| | is 1:1 (NMR), melts around 161° C. |
| Mesylate | One crystalline phase, designated as form 9, was identified in |
| | the salt screen. Form 9: crystalline, unsolvated, stoichiometry |
| | is 1:1 (NMR), melts around 190° C. (DSC). |
| Pamoate | One crystalline phase, designated as form 10, was identified in |
| | the salt screen. Form 10: crystalline, unsolvated, stoichiometry |
| | is 1:1 (NMR), melts around 225° C. |

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A salt of Compound 1, wherein Compound 1 is represented by the following structural formula: 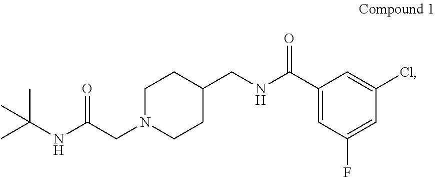 [SMILES: CC(C)(C)NC(=O)CN1CCC(CNC(=O)c2cc(F)cc(Cl)c2)CC1]
and wherein the salt is selected from the group consisting of acetate salt, adipate salt, alginate salt, ascorbate salt, asparatate salt, besylate salt, benzoate salt, citrate salt, cyclamate salt, edisylate salt, esylate salt, isethionate salt, fumarate salt, gentisate salt, gluconate salt, glucuronate salt, glutamate salt, glutarate salt, ketoglutarate salt, glycolate salt, hippurate salt, lactobionate salt, maleate salt, malate salt, malonate salt, mesylate salt, napadisylate salt, napsylate salt, oleate salt, oroate salt, oxalate salt, pamoate salt, phosphate salt, sebacate salt, succinate salt and tartrate salt.

2. A crystalline form of a salt of Compound 1, wherein Compound 1 is represented by the following structural formula: 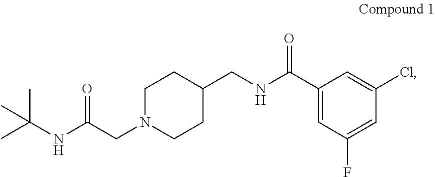 [SMILES: CC(C)(C)NC(=O)CN1CCC(CNC(=O)c2cc(F)cc(Cl)c2)CC1]
and wherein the salt is selected from the group consisting of adipate salt, besylate salt, cyclamate salt, edisylate salt, esylate salt, fumarate salt, glutarate salt, glycolate salt, inappendiculate salt, napsylate salt, orotate salt, oxalate salt, maleate salt, malonate salt, mesylate salt, pamoate salt, phosphate salt, and sebacate salt.

3. The crystalline form of claim 2, wherein the salt is edisylate salt, and wherein the crystalline form is Form 1 characterized by an X-ray powder diffraction pattern (XRPD pattern) that includes at least one peak at the diffraction angle (° 2θ) selected from the group of peaks listed in Table 1.

4-5. (canceled)

6. The crystalline form of claim 2, wherein the salt is esylate salt, and wherein the crystalline form is Form 2 characterized by an XRPD pattern that includes at least one peak at the diffraction angle (° 2θ) selected from the group of peaks listed in Table 2.

7-8. (canceled)

9. The crystalline form of claim 2, wherein the salt is glutarate salt, and wherein the crystalline form is Form 3 characterized by an XRPD pattern that includes at least one peak at the diffraction angle (° 2θ) selected from the group of peaks listed in Table 3.

10-11. (canceled)

12. The crystalline form of claim 2, wherein the salt is napadisylate salt, and wherein the crystalline form is Form 4 characterized by an X-ray powder diffraction pattern (XRPD pattern) that includes at least one peak at the diffraction angle (° 2θ) selected from the group of peaks listed in Table 4.

13-14. (canceled)

15. The crystalline form of claim 2, wherein the salt is napsylate salt, and wherein the crystalline form is Form 5 characterized by an XRPD pattern that includes at least one peak at the diffraction angle (° 2θ) selected from the group of peaks listed in Table 5.

16-17. (canceled)

18. The crystalline form of claim 2, wherein the salt is orotate salt, and wherein the crystalline form is Form 6 characterized by an XRPD pattern that includes at least one peak at the diffraction angle (° 2θ) selected from the group of peaks listed in Table 6.

19-20. (canceled)

21. The crystalline form of claim 2, wherein the salt is maleate salt, and wherein the crystalline form is Form 7 characterized by an XRPD pattern that includes at least one peak at the diffraction angle (° 2θ) selected from the group of peaks listed in Table 7.

22-24. (canceled)

25. The crystalline form of claim 2, wherein the salt is malonate salt, and wherein the crystalline form is Form 8 characterized by an XRPD pattern that includes at least one peak at the diffraction angle (° 2θ) selected from the group of peaks listed in Table 8.

26-28. (canceled)

29. The crystalline form of claim 2, wherein the salt is mesylate salt, and wherein the crystalline form is Form 9 characterized by an XRPD pattern that includes at least one peak at the diffraction angle (° 2θ) selected from the group of peaks listed in Table 9.

30-32. (canceled)

33. The crystalline form of claim 2, wherein the salt is pamoate salt, and wherein the crystalline form is Form 10 characterized by an XRPD pattern that includes at least one peak at the diffraction angle (° 2θ) selected from the group of peaks listed in Table 10.

34-36. (canceled)

37. The crystalline form of claim 2, wherein the salt is adipate salt, and wherein the crystalline form is Form 11 characterized by an XRPD pattern that includes at least one peak at the diffraction angle (° 2θ) selected from the group of peaks listed in Table 11.

38-39. (canceled)

40. The crystalline form of claim 2, wherein the salt is besylate salt, and wherein the crystalline form is Form 12 characterized by an XRPD pattern that includes at least one peak at the diffraction angle (° 2θ) selected from the group of peaks listed in Table 12.

41-42. (canceled)

43. The crystalline form of claim 2, wherein the salt is cyclamate salt, and wherein the crystalline form is Form 13 characterized by an XRPD pattern that includes at least one peak at the diffraction angle (° 2θ) selected from the group of peaks listed in Table 13.

44-45. (canceled)

46. The crystalline form of claim 2, wherein the salt is fumarate salt, and wherein the crystalline form is Form 14 characterized by an XRPD pattern that includes at least one peak at the diffraction angle (° 2θ) selected from the group of peaks listed in Table 14.

47-48. (canceled)

49. The crystalline form of claim 2, wherein the salt is glycolate salt, and wherein the crystalline form is Form 15 characterized by an XRPD pattern that includes at least one peak at the diffraction angle (° 2θ) selected from the group of peaks listed in Table 15.

50-51. (canceled)

52. The crystalline form of claim 2, wherein the salt is oxalate salt, and wherein the crystalline form is Form 16 characterized by an XRPD pattern that includes at least one peak at the diffraction angle (° 2θ) selected from the group of peaks listed in Table 16.

53-54. (canceled)

55. The crystalline form of claim 2, wherein the salt is phosphate salt, and wherein the crystalline form is Form 17 characterized by an XRPD pattern that includes at least one peak at the diffraction angle (° 2θ) selected from the group of peaks listed in Table 17.

56-57. (canceled)

58. The crystalline form of claim 2, wherein the salt is sebacate salt, and wherein the crystalline form is Form 18 characterized by an XRPD pattern that includes at least one peak at the diffraction angle (° 2θ) selected from the group of peaks listed in Table 18.

59-60. (canceled)

61. A pharmaceutical composition comprising the salt of claim 1 and a pharmaceutically acceptable carrier.

62-65. (canceled)

66. A method of treating a neurological disorder comprising administering to a subject in need thereof the salt of claim 1.

67-69. (canceled)

70. A method of treating a movement disorder in a subject in need thereof, said method comprising administering to said subject the salt of claim 1.

71. The method of claim 66, wherein the neurological disorder is tremor.

72. The method of claim 71, wherein the tremor is essential tremor.
